(19) United States
(12) Patent Application Publication
(10) Pub. No.: US 2008/0318234 A1
(43) Pub. Date: Dec. 25, 2008

(54) Compositions and methods for diagnosing and treating cancer

(76) Inventor: Xinhao Wang, Fremont, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/081,484

(22) Filed: Apr. 16, 2008

Related U.S. Application Data
(60) Provisional application No. 60/907,761, filed on Apr. 16, 2007.

(51) Int. Cl. C12Q 1/68; C40B 40/08

(52) U.S. Cl. 435/6; 506/17

(57) ABSTRACT

The present invention relates to compositions and methods for treating, characterizing, and diagnosing cancer. In particular, the present invention provides gene expression profiles associated with solid tumor stem cells, as well as novel stem cell cancer gene signatures useful for the diagnosis, characterization, prognosis and treatment of solid tumor stem cells.

### CROSS REFERENCE TO RELATED APPLICATIONS

This application claims the benefit of U.S. Prov. Appl. No. 60/907,761, filed Apr. 16, 2007, which is herein incorporated by reference. This application is also related to U.S. application Ser. No. 10/864,207 filed Jun. 9, 2004, U.S. Appl. Nos. 60/477,228 and 60/477,235, both filed Jun. 9, 2003, and U.S. Appl. No. 60/690,003, filed Jun. 13, 2005 each of which are herein incorporated by reference in their entirety.

Table 34, filed herewith under 37 C.F.R. §§ 1.52 and 1.58, written in file “C4_{—}6_{—}9_{—}3.txt,” 2,990,080 bytes, created on Apr. 10, 2007, is submitted in two identical sets of compact discs labeled “Copy 1” and “Copy 2,” which are herein incorporated by reference. Each compact disc contains one file in ASCII format and the compact discs were prepared in IBM-PC machine format and are compatible with the MS-Windows operating system.

### BACKGROUND OF THE INVENTION

1. Field of the Invention

The present invention relates to compositions and methods for treating, characterizing, and diagnosing cancer. In particular, the present invention provides gene expression profiles associated with solid tumor stem cells, as well as novel stem cell cancer markers useful for the diagnosis, characterization, and treatment of solid tumor stem cells. The invention further provides distinct tumor stem cell gene signatures useful for categorizing tumors into subclasses and for diagnosis, prognosis, and treatment of cancer.

2. Background Art

Breast cancer is the most common female malignancy in most industrialized countries, as it is estimated to affect about 10% of the female population during their lifespan. Although its mortality has not increased along with its incidence, due to earlier diagnosis and improved treatment, it is still one of the predominant causes of death in middle-aged women. Despite earlier diagnosis of breast cancer, about 1-5% of women with newly diagnosed breast cancer have a distant metastasis at the time of the diagnosis. In addition, approximately 50% of the patients with local disease who are primarily diagnosed eventually relapse with the metastasis. Eighty-five percent of these recurrences take place within the first five years after the primary manifestation of the disease.

On presentation, most patients with metastatic breast cancer have only one or two organ systems involved. As the disease progresses over time, multiple sites usually become involved. Indeed, metastases can be found in nearly every organ of the body at autopsy. The most common sites of metastatic involvement observed are locoregional recurrences in the skin and soft tissues of the chest wall, as well as in axilla, and supraclavicular area. The most common site for distant metastasis is the bone (30-40% of distant metastasis), followed by lung and liver. Metastatic breast cancer is generally considered to be an incurable disease. However, the currently available treatment options often prolong the disease-free state and overall survival rate, as well as increase the quality of the life. The median survival from the manifestation of distant metastases is about three years.

Current methods of diagnosing and staging breast cancer include the tumor-node-metastasis (TNM) system that relies on tumor size, tumor presence in lymph nodes, and the presence of distant metastases as described in the American Joint Committee on Cancer: AJCC Cancer Staging Manual. Philadelphia, Pa.: Lippincott-Raven Publishers, 5th ed., 1997, pp 171-180, and in Harris, J R: “Staging of breast carcinoma” in Harris, J. R., Hellman, S., Henderson, I. C., Kinne D. W. (eds.): Breast Diseases. Philadelphia, Lippincott, 1991. These parameters are used to provide a prognosis and select an appropriate therapy. The morphologic appearance of the tumor can also be assessed but because tumors with similar histopathologic appearance can exhibit significant clinical variability, this approach has serious limitations. Finally assays for cell surface marker can be used to divide certain tumors types into subclasses. For example, one factor considered in the prognosis and treatment of breast cancer is the presence of the estrogen receptor (ER) as ER-positive breast cancers typically respond more readily to hormonal therapies such as tamoxifen than ER-negative tumors. Yet these analyses, though useful, are only partially predictive of the clinical behavior of breast tumors, and there is much phenotypic diversity present in breast cancers that current diagnostic tools fail to detect.

Traditional modes of cancer therapy include radiation therapy, chemotherapy, and hormonal therapy. Yet because of the difficulty in predicting the clinical course of early stage breast cancer from standard clinical and pathologic features, current practice is to offer systemic chemotherapy to most women even though the majority of these women would have good outcome in the absence of chemotherapy. Chemotherapy has severe side effects and itself carries a 1% mortality rate, and thus unnecessary suffering and deaths could be avoided if patients could be divided into high and low risk subgroups. Thus, there exists a need for improved methods to classifying tumors for better prognosis and treatment selection.

Furthermore, although current therapies can often prolong the disease-free state and overall survival when used on high-risk patients, they are limited by their lack of specificity and the emergence of treatment-resistant cancer cells. Approximately two thirds of people diagnosed with cancer will die of their cancer within five years. Thus there is a great call for the identification of additional genes that can serve as selective therapies for the treatment of cancer.

Colorectal cancer is the third most common cancer and the fourth most frequent cause of cancer deaths worldwide. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Colorectal carcinoma has a tendency to invade locally by circumferential growth and for lymphatic, hematogenous, transperitoneal, and perineural spread. The most common site of extralymphatic involvement is the liver, with the lungs the most frequently affected extra-abdominal organ. Other sites of hematogenous spread include the bones, kidneys, adrenal glands, and brain.

The current staging system for colorectal cancer is based on the degree of tumor penetration through the bowel wall and the presence or absence of nodal involvement. This staging system is defined by three major Duke's classifications: Duke's A disease is confined to submucosa layers of colon or rectum; Duke's B disease has tumors that invade through muscularis propria and can penetrate the wall of the colon or rectum; and Duke's C disease includes any degree of bowel wall invasion with regional lymph node metastasis.

Surgical resection is highly effective for early stage colorectal cancers, providing cure rates of 95% in Duke's A and 75% in Duke's B patients. The presence of positive lymph node in Duke's C disease predicts a 60% likelihood of recurrence within five years. Treatment of Duke's C patients with a post surgical course of chemotherapy reduces the recurrence rate to 40%-50%, and is now the standard of care for these patients. Because of the relatively low rate of reoccurrence, the benefit of post surgical chemotherapy in Duke' B has been harder to detect and remains controversial. However, the Duke's B classification is imperfect as approximately 20-30% of these patients behave more like Duke's C and relapse within five years. Thus there is a clear need to identify better prognostic factors for selecting Duke's B patients that are likely to relapse and would benefit from therapy.

During normal animal development, cells of most or all tissues are derived from normal precursors, called stem cells (Morrison et al., Cell 88(3): 287-98 (1997); Morrison et al., Curr. Opin. Immunol. 9(2): 216-21 (1997); Morrison et al., Annu. Rev. Cell. Dev. Biol. 11: 35-71 (1995)). Stem cells are cells that: (1) have extensive proliferative capacity; 2) are capable of asymmetric cell division to generate one or more kinds of progeny with reduced proliferative or developmental potential; and (3) are capable of symmetric cell divisions for self-renewal or self-maintenance. In adult animals, some cells (including cells of the blood, gut, breast ductal system, and skin) are constantly replenished from a small population of stem cells in each tissue. The best-known example of adult cell renewal by the differentiation of stem cells is the hematopoietic system where developmentally immature precursors (hematopoietic stem and progenitor cells) respond to molecular signals to form the varied blood and lymphoid cell types.

Solid tumors are composed of heterogeneous cell populations. For example, breast cancers are a mixture of cancer cells and normal cells, including mesenchymal (stromal) cells, inflammatory cells, and endothelial cells. Classic models of cancer hold that phenotypically distinct cancer cell populations all have the capacity to proliferate and give rise to a new tumor. In the classical model, tumor cell heterogeneity results from environmental factors as well as ongoing mutations within cancer cells resulting in a diverse population of tumorigenic cells. This model rests on the idea that all populations of tumor cells would have some degree of tumorigenic potential. (Pandis et al., Genes, Chromosomes & Cancer 12:122-129 (1998); Kuukasjrvi et al., Cancer Res. 57: 1597-1604 (1997); Bonsing et al., Cancer 71: 382-391 (1993); Bonsing et al., Genes Chromosomes & Cancer 82: 173-183 (2000); Beerman H et al., Cytometry. 12(2): 147-54 (1991); Aubele M & Werner M, Analyt. Cell. Path. 19: 53 (1999); Shen L et al., Cancer Res. 60: 3884 (2000).).

An alternative model for the observed solid tumor cell heterogeneity is that solid tumors result from a “solid tumor stem cell” (or “cancer stem cell” from a solid tumor) that subsequently undergoes chaotic development through both symmetric and asymmetric rounds of cell divisions. In this stem cell model, solid tumors contain a distinct and limited (possibly even rare) subset of cells that share the properties of normal “stem cells”, in that they extensively proliferate and efficiently give rise both to additional solid tumor stem cells (self-renewal) and to the majority of tumor cells of a solid tumor that lack tumorigenic potential. Indeed, mutations within a long-lived stem cell population can initiate the formation of cancer stem cells that underlie the growth and maintenance of tumors and whose presence contributes to the failure of current therapeutic approaches.

Although great strides have been made understanding the genetic changes that lead to cancer (e.g. breast cancer and colorectal cancer), the lack of reliable tumor assay for de novo human cancer cells has hindered the ability to understand the effects of these mutations at the cellular level. Also, the lack of identified cancer markers for solid tumor stem cells has hindered the development of diagnostics and therapeutics for cancer patients (e.g. breast cancer patients). As such, what is needed is a reliable tumor assay as well as the identification of cancer markers for solid tumor stem cells.

### BRIEF SUMMARY OF THE INVENTION

The present invention relates to compositions and methods for treating, characterizing, and diagnosing cancer. In particular, the present invention provides novel stem cell cancer markers useful for the diagnosis, characterization, and treatment of solid tumor stem cells. The present invention further provides gene signatures derived from solid tumor stem cell markers that, when detected in a tumor sample as a gene expression profile, act as significant predictors of poor clinical outcome, including high risk of metastasis and death.

### BRIEF DESCRIPTION OF THE DRAWINGS/FIGURES

FIG. 1 shows Kaplan-Meier plots for survival and metastasis generated from 295 breast cancer patients from the Netherlands Cancer Institute (NKI dataset). Patients were separated by the correlation (<=average (upper line) or >average (lower line)) of their gene expression profile with cancer stem cell signature 1. The difference between the two curves in each plot is statistically significant (P≦0.0001) by logrank test.

FIG. 2 shows Kaplan-Meier plots for survival generated from the breast cancer patients from the GSE 1456 and 3494 datasets. Patients were separated by the correlation (<=average (upper line) or >average (lower line)) of their gene expression profile with cancer stem cell signature 1. The difference between the two curves is statistically significant (P≦0.0001) by logrank test.

FIG. 3 shows Kaplan-Meier plots for survival and metastasis generated from 295 breast cancer patients from the Netherlands Cancer Institute (NKI dataset). Patients were separated by the correlation (<=average (upper line) or >average (lower line)) of their gene expression profile with cancer stem cell signature 2. The difference between the two curves in each plot is statistically significant (P≦0.0001) by logrank test.

FIG. 4 shows Kaplan-Meier plots for survival generated from the breast cancer patients from the GSE 1456 and 3494 datasets. Patients were separated by the correlation (<=average (upper line) or >average (lower line)) of their gene expression profile with cancer stem cell signature 2. The difference between the two curves is statistically significant (P≦0.0001) by logrank test.

FIG. 5 shows Kaplan-Meier plots for survival generated from the breast cancer patients from the GSE 1456 and 3494 datasets. Patients were separated by the correlation (<=average (upper line) or >average (lower line)) of their gene expression profile with cancer stem cell signature 3. The difference between the two curves is statistically significant (P=0.0023) by logrank test.

FIG. 6 shows Kaplan-Meier plots for survival and metastasis generated from NKI dataset. Patients were separated by the correlation (<=average (upper line) or >average (lower line)) of their gene expression profile with cancer stem cell signature 4. The difference between the two curves is statistically significant, metastasis (P=0.0016) and death (P≦0.0001), by logrank test.

FIG. 7 shows Kaplan-Meier plots for survival and relapse generated from the breast cancer patients from the GSE 1456 dataset. Patients were separated by the correlation (<=average (upper line) or >average (lower line)) of their gene expression profile with cancer stem cell signature 4. The difference between the two curves in each plot is statistically significant (P≦0.0001) by logrank test.

FIG. 8 shows Kaplan-Meier plots for relapse and distance metastasis generated from the breast cancer patients from the GSE 2990 dataset. Patients were separated by the correlation (<=average (upper line) or >average (lower line)) of their gene expression profile with cancer stem cell signature 4. The difference between the two curves in each plot is statistically significant (P≦0.0001) by logrank test.

### DETAILED DESCRIPTION OF THE INVENTION

### General Description

This invention is based on the discovery of solid tumor stem cells (also referred to as cancer stem cells from a solid tumor) as a distinct and limited subset of cells within the heterogeneous cell population of established solid tumors. These cancer stem cells share the properties of normal stem cells in that they extensively proliferate and efficiently give rise both to additional solid tumor stem cells (self-renewal) and to the majority of tumor cells of a solid tumor that lack tumorigenic potential. Identification of cancer stem cells from solid tumors relied on their expression of a unique pattern of cell-surface receptors that could be used to isolate them from the bulk of non-tumorigenic tumor cells and on the assessment of their properties of self-renewal and proliferation in culture and in xenograft animal models. An ESA+; CD44+; CD24−/low; Lineage-population greater than 50-fold enriched for the ability to form tumors relative to unfractionated tumor cells was discovered (Al-Hajj et al., 2003; U.S. Appl. Publ. Nos. 20020119565 and 20040037815, herein incorporated by reference in their entireties).

The present invention relates to compositions and methods for treating, characterizing and diagnosing cancer. In particular, the present invention provides cancer stem cell gene signatures associated with solid tumor stem cells useful for the diagnosis, characterization, and treatment of solid tumor stem cells. Suitable cancer stem cell markers used to identify cancer stem cell signature and that can be targeted (e.g. for diagnostic or therapeutic purposes) are the genes and peptides encoded by the genes that are differentially expressed in solid tumor stem cells as shown in Tables 9A-9N2. To identify solid tumor stem cell markers, tumorigenic breast cancer cells from 6 patients and non-tumorigenic breast cancer cells from 3 patients, 3 samples of normal breast epithelial cells (GEO accession: GSE6883), normal hematopoietic stem cells (HSCs) and 3 tumorigenic and non-tumorigenic colon cancer cells were analyzed for differential expression.

In certain embodiments, the present invention provides methods of determining the presence or absence of a solid tumor stem cell gene expression profile, comprising: a) providing a tissue sample from a subject, and b) detecting genes of a solid tumor stem cell gene signature in the tissue sample under conditions such that the presence or absence of a solid tumor stem cell gene expression profile in the tissue sample is determined. In certain embodiments, the methods of the present invention further comprise c) providing a prognosis to the subject.

In certain embodiments, detecting a solid tumor stem cell gene expression profile comprises determining the expression levels of polynucleotides comprising a cancer stem cell gene signature. In certain embodiments, the detecting a cancer stem cell gene profile comprises detecting mRNA expression of polynucleotides comprising a cancer stem cell gene signature. In some embodiments, the detection of mRNA expression is via Northern blot. In some embodiments, the detection of mRNA expression is via RT-PCR, real-time PCR or quantitative PCR using primer sets that specifically amplify the polynucleotides comprising the cancer stem cell signature. In certain embodiments, the detection of mRNA comprises exposing a sample to nucleic acid probes complementary to polynucleotides comprising a cancer stem cell gene signature. In some embodiments, the mRNA of the sample is converted to cDNA prior to detection. In some embodiments, the detection of mRNA is via microarrays that comprise a cancer stem cell gene signature.

In certain embodiments, the detecting comprises detecting polypeptides encoded by polynucleotides comprising a cancer stem cell gene signature. In some embodiments, the detection of polypeptide expression comprises exposing a sample to antibodies specific to the polypeptides and detecting the binding of the antibodies to the polypeptides by, for example, quantitative immunofluorescence or ELISA. Other detection means are known to one of ordinary skill in the art see e.g., U.S. Pat. No. 6,057,105.

In certain embodiments, reagents and methods for predicting a subject's clinical outcome (including, but not limited to, metastasis and death) are provided using the cancer stem cell gene signatures of the present invention. Cancer stem cell gene signatures comprising identified cancer stem cell markers are provided that are predictive of metastasis and overall survival and can thus be used to classify tumors into low and high-risk subclasses and further provide a diagnosis, provide a prognosis, select a therapy, and monitor a therapy. In certain embodiments, a method of classifying a tumor comprises: a) providing a tumor sample, for example by obtaining a tumor biopsy from a subject; b) determining expression or activity of at least one polynucleotide or polypeptide selected from a solid tumor stem cell gene signature; and c) classifying the tumor as belonging to a high risk or low risk tumor class based on the results of b). In certain embodiments, the method further comprises providing a diagnosis, prognosis, selecting a therapy, or monitoring a therapy.

According to certain of the inventive methods, the presence or amount of a gene product, e.g., a polypeptide or a nucleic acid, encoded by a solid tumor stem cell gene is detected in a sample derived from a subject (e.g. a sample of tissue or cells obtained from a tumor or a blood sample obtained from a subject). In certain embodiments, the subject is a human. In some embodiments, the subject is an individual who has or can have a tumor. The sample can be subjected to a number of processing steps prior to or in the course of detection.

In certain embodiments, the present invention provides kits for detecting solid tumor stem cell gene expression profiles in a subject, comprising: a) at least one reagent capable of specifically detecting at least one gene of a cancer stem cell signature in a tissue or cell sample from a subject, and b) instructions for using the reagent(s) for detecting the presence or absence of a solid tumor stem cell gene expression profile in the tissue sample. In some embodiments, the at least one reagent comprises nucleic acid probes complementary to mRNA of at least one gene of a cancer stem cell gene signature. In some embodiments, the at least one reagent comprises antibodies or antibody fragments that specifically bind to at least one gene product of a cancer stem cell signature.

Examples of solid tumors from which samples or solid tumor stem cells can be isolated or enriched for use in accordance with the invention include, but are not limited to, sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The invention is applicable to sarcomas and epithelial cancers, such as ovarian cancers and breast cancers.

The present invention thus provides for the first time cancer stem cell gene signatures comprising cancer stem cell markers that are predictive of clinical outcome including metastasis and overall survival. These cancer stem cell signatures shown in Tables 9-16 are established as predictive of a poor prognosis. In some embodiments of the present invention, the cancer stem cell signatures are used clinically to classify tumors as low or high risk and to assign a tumor to a low or high risk category. The cancer stem cell signatures can further be used to provide a diagnosis, prognosis, and select a therapy based on the classification of a tumor as low or high risk as well as to monitor the diagnosis, prognosis, and/or therapy over time. In some embodiments, the cancer stem cell signatures can be used experimentally to test and assess lead compounds including, for example, small molecules, siRNAs, gene therapy, and antibodies for the treatment of cancer.

Other features, objects, and advantages of the invention will be apparent from the detailed description below. Additional guidance is provided in published PCT patent application WO 02/12447 by the Regents of the University of Michigan and PCT patent application PCT/US02/39191 by the Regents of the University of Michigan, both of which are incorporated herein by reference.

### DEFINITIONS

To facilitate an understanding of the present invention, a number of terms and phrases are defined below:

The term “antibody” is used to mean an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The term “antibody” encompasses polyclonal antibodies, monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

An “Fv antibody” refers to the minimal antibody fragment that contains a complete antigen-recognition and -binding site either as two-chains, in which one heavy and one light chain variable domain form a non-covalent dimer, or as a single-chain (scFv), in which one heavy and one light chain variable domain are covalently linked by a flexible peptide linker so that the two chains associate in a similar dimeric structure. In this configuration the complementary determining regions (CDRs) of each variable domain interact to define the antigen-binding specificity of the Fv dimer. Alternatively a single variable domain (or half of an Fv) can be used to recognize and bind antigen, although generally with lower affinity.

A “monoclonal antibody” refers to homogenous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Examples of methods that can be used to generate humanized antibodies are described in U.S. Pat. No. 5,225,539 to Winter et al. (herein incorporated by reference).

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

“Hybrid antibodies” are immunoglobulin molecules in which pairs of heavy and light chains from antibodies with different antigenic determinant regions are assembled together so that two different epitopes or two different antigens can be recognized and bound by the resulting tetramer.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

“Enriched”, as in an enriched population of cells, can be defined phenotypically based upon the increased number of cells having a particular marker (e.g. as shown in Tables 9A-9N2) in a fractionated set of cells as compared with the number of cells having the marker in the unfractionated set of cells. However, the term “enriched can be defined functionally by tumorigenic function as the minimum number of cells that form tumors at limit dilution frequency in test mice. For example, if 500 tumor stem cells form tumors in 63% of test animals, but 5000 unfractionated tumor cells are required to form tumors in 63% of test animals, then the solid tumor stem cell population is 10-fold enriched for tumorigenic activity. The stem cell cancer markers of the present invention can be used to generate enriched populations of cancer stem cells. In some embodiments, the stem cell population is enriched at least 1.4 fold relative to unfractioned tumor cells (e.g. 1.4 fold, 1.5 fold, 2 fold, 5 fold, 10 fold, 12.5 fold, 15 fold, 20 fold, 25 fold, 50 fold, 100 fold).

“Isolated” in regard to cells, refers to a cell that is removed from its natural environment (such as in a solid tumor) and that is isolated or separated, and is at least about 30%, 50%, 75%, 90%, 92.5%, 95%, 96%, 97%, 98%, or 99% free, from other cells with which it is naturally present, but which lack the marker based on which the cells were isolated. The stem cell cancer markers of the present invention can be used to generate isolated populations of cancer stem cells.

As used herein, the terms “low levels”, “decreased levels”, “low expression”, “reduced expression” or “decreased expression” in regards to gene expression are used herein interchangeably to refer to expression of a gene in a cell or population of cells, particularly a cancer stem cell or population of cancer stem cells, at levels less than the expression of that gene in a second cell or population of cells, for example normal breast epithelial cells. “Low levels” of gene expression refers to expression of a gene in a cancer stem cell or population of cancer stem cells at levels: 1) half that or below expression levels of the same gene in normal breast epithelial cells and 2) at the lower limit of detection using conventional techniques. “Low levels” of gene expression can be determined by detecting decreased to nearly undetectable amounts of a polynucleotide (mRNA, cDNA, etc.) in cancer stem cells compared to normal breast epithelium by, for example, quantitative RT-PCR or microarray analysis. Alternatively “low levels” of gene expression can be determined by detecting decreased to nearly undetectable amounts of a protein in cancer stem cells compared to normal breast epithelium by, for example, ELISA, Western blot, quantitative immunofluorescence, etc.

The terms “high levels”, “increased levels”, “high expression”, “increased expression” or “elevated levels” in regards to gene expression are used herein interchangeably to refer to expression of a gene in a cell or population of cells, particularly a cancer stem cell or population of cancer stem cells, at levels higher than the expression of that gene in a second cell or population of cells, for example normal breast epithelial cells. “Elevated levels” of gene expression refers to expression of a gene in a cancer stem cell or population of cancer stem cells at levels twice that or more of expression levels of the same gene in normal breast epithelial cells. “Elevated levels” of gene expression can be determined by detecting increased amounts of a polynucleotide (mRNA, cDNA, etc.) in cancer stem cells compared to normal breast epithelium by, for example, quantitative RT-PCR or microarray analysis. Alternatively “elevated levels” of gene expression can be determined by detecting increased amounts of a protein in cancer stem cells compared to normal breast epithelium by, for example, ELISA, Western blot, quantitative immunofluorescence, etc.

The term “undetectable levels” or “loss of expression” in regards to gene expression as used herein refers to expression of a gene in a cell or population of cells, particularly a cancer stem cell or population of cancer stem cells, at levels that cannot be distinguished from background using conventional techniques such that no expression is identified. “Undetectable levels” of gene expression can be determined by the inability to detect levels of a polynucleotide (mRNA, cDNA, etc.) in cancer stem cells above background by, for example, quantitative RT-PCR or microarray analysis. Alternatively “undetectable levels” of gene expression can be determined by the inability to detect levels of a protein in cancer stem cells above background by, for example, ELISA, Western blot, immunofluorescence, etc.

As used herein, the term “antibody-immunoadhesin chimera” comprises a molecule that combines at least one binding domain of an antibody with at least one immunoadhesin. Examples include, but are not limited to, the bispecific CD4-IgG chimeras described in Berg et al., PNAS (USA) 88:4723-4727 (1991) and Charnow et al., J. Immunol., 153:4268 (1994), both of which are hereby incorporated by reference.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The term “epitope” as used herein refers to that portion of an antigen that makes contact with a particular antibody. When a protein or fragment of a protein is used to immunize a host animal, numerous regions of the protein can induce the production of antibodies which bind specifically to a given region or three-dimensional structure on the protein; these regions or structures are referred to as “antigenic determinants”. An antigenic determinant can compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer can also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental exposure, previous incidents of cancer, preexisting non-cancer diseases, and lifestyle.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers can be characterized by the identification of the expression of one or more cancer marker genes, including but not limited to, the cancer markers disclosed herein.

The terms “cancer stem cell”, “tumor stem cell”, or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells”, “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur. The solid tumor stem cells of the present invention differ from the “cancer stem line” provided by U.S. Pat. No. 6,004,528. In that patent, the “cancer stem line” is defined as a slow growing progenitor cell type that itself has few mutations but which undergoes symmetric rather than asymmetric cell divisions as a result of tumorigenic changes that occur in the cell's environment. This “cancer stem line” hypothesis thus proposes that highly mutated, rapidly proliferating tumor cells arise largely as a result of an abnormal environment, which causes relatively normal stem cells to accumulate and then undergo mutations that cause them to become tumor cells. U.S. Pat. No. 6,004,528 proposes that such a model can be used to enhance the diagnosis of cancer. The solid tumor stem cell model is fundamentally different than the “cancer stem line” model and as a result exhibits utilities not offered by the “cancer stem line” model. First, solid tumor stem cells are not “mutationally spared”. The “mutationally spared cancer stem line” described by U.S. Pat. No. 6,004,528 can be considered a pre-cancerous lesion, while the solid tumor stem cells described by this invention are cancer cells that themselves contain the mutations that are responsible for tumorigenesis. That is, the solid tumor stem cells (“cancer stem cells”) of the invention would be included among the highly mutated cells that are distinguished from the “cancer stem line” in U.S. Pat. No. 6,004,528. Second, the genetic mutations that lead to cancer can be largely intrinsic within the solid tumor stem cells as well as being environmental. The solid tumor stem cell model predicts that isolated solid tumor stem cells can give rise to additional tumors upon transplantation (thus explaining metastasis) while the “cancer stem line” model would predict that transplanted “cancer stem line” cells would not be able to give rise to a new tumor, since it was their abnormal environment that was tumorigenic. Indeed, the ability to transplant dissociated, and phenotypically isolated human solid tumor stem cells to mice (into an environment that is very different from the normal tumor environment), where they still form new tumors, distinguishes the present invention from the “cancer stem line” model. Third, solid tumor stem cells likely divide both symmetrically and asymmetrically, such that symmetric cell division is not an obligate property. Fourth, solid tumor stem cells can divide rapidly or slowly, depending on many variables, such that a slow proliferation rate is not a defining characteristic.

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the terms “stem cell cancer marker(s)”, “cancer stem cell marker(s)”, “tumor stem cell marker(s)”, or “solid tumor stem cell marker(s)” refer to a gene or genes or a protein, polypeptide, or peptide expressed by the gene or genes whose expression level, alone or in combination with other genes, is correlated with the presence of tumorigenic cancer cells compared to non-tumorigenic cells. The correlation can relate to either an increased or decreased expression of the gene (e.g. increased or decreased levels of mRNA or the peptide encoded by the gene).

A “gene profile,” “gene pattern,” “expression pattern,” “expression profile,” “gene expression profile” or grammatical equivalents refer to identified expression levels of at least one polynucleotide or protein expressed in a biological sample and thus refer to a specific pattern of gene expression that provides a unique identifier of a biological sample, for example, a breast or colon cancer pattern of gene expression obtained by analyzing a breast or colon cancer sample in comparison to a reference sample will be referred to as a “breast cancer gene profile” or a “colon cancer expression pattern”. “Gene patterns” can be used to diagnose a disease, make a prognosis, select a therapy, and/or monitor a disease or therapy after comparing the gene pattern to a cancer stem cell gene signature.

The terms “cancer stem cell gene signature”, “tumor stem cell gene signature”, “cancer stem cell signature”, “tumor stem cell signature”, “tumorigenic gene signature”, “TG gene signature” and grammatical equivalents are used interchangeably herein to refer to gene signatures comprising genes differentially expressed in cancer stem cells compared to other cells or population of cells, for example normal breast epithelial tissue. In some embodiments, the cancer stem cell gene signatures comprise genes differentially expressed in cancer stem cells versus normal breast epithelium by a fold change, for example by 2-fold reduced and/or elevated expression, and further limited by using a statistical analysis such as, for example, by the P value of a t-test across multiple samples. In some embodiments, the genes differentially expressed in cancer stem cells are divided into cancer stem cell gene signatures based on the correlation of their expression with a chosen gene in combination with their fold or percentage expression change. Cancer stem cell signatures can be predictive both retrospectively and prospectively of an aspect of clinical variability, including but not limited to metastasis and death.

As used herein, the term “a reagent that specifically detects expression levels” refers to reagents used to detect the expression of one or more genes (e.g., including but not limited to, the cancer markers of the present invention). Examples of suitable reagents include but are not limited to, nucleic acid probes capable of specifically hybridizing to the gene of interest, aptamers, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous control” refers to measuring the level of expression of a gene (e.g., the level of mRNA or protein) relative to the level in a non-cancerous control sample. Gene expression can be measured using any suitable method, including but not limited to, those described herein.

As used herein, the term “detecting a change in gene expression in a cell sample in the presence of said test compound relative to the absence of said test compound” refers to measuring an altered level of expression (e.g., increased or decreased) in the presence of a test compound relative to the absence of the test compound. Gene expression can be measured using any suitable method.

As used herein, the term “instructions for using said kit for detecting cancer in said subject” includes instructions for using the reagents contained in the kit for the detection and characterization of cancer in a sample from a subject.

As used herein, “providing a diagnosis” or “diagnostic information” refers to any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a tumor), information related to the nature or classification of a tumor, information related to prognosis and/or information useful in selecting an appropriate treatment. Selection of treatment can include the choice of a particular chemotherapeutic agent or other treatment modality such as surgery, radiation, etc., a choice about whether to withhold or deliver therapy, etc.

As used herein, the terms “providing a prognosis”, “prognostic information”, or “predictive information” refer to providing information regarding the impact of the presence of cancer (e.g., as determined by the diagnostic methods of the present invention) on a subject's future health (e.g., expected morbidity or mortality, the likelihood of getting cancer, and the risk of metastasis).

The term “low risk” in regards to tumors or to patients diagnosed with cancer refers to a tumor or patient with a lower probability of metastasis and/or lower probability of causing death or dying within about five years of first diagnosis than all the tumors or patients within a given population.

The term “high risk” in regards to tumors or to patients diagnosed with cancer refers to a tumor or patient with a higher probability of metastasis and/or higher probability of causing death or dying within about five years of first diagnosis than all the tumors or patients within a given population.

As used herein, the term “post surgical tumor tissue” refers to cancerous tissue (e.g., biopsy tissue) that has been removed from a subject (e.g., during surgery).

As used herein, the term “subject diagnosed with a cancer” refers to a subject who has been tested and found to have cancerous cells. The cancer can be diagnosed using any suitable method, including but not limited to, biopsy, x-ray, blood test, and the diagnostic methods of the present invention.

As used herein, the terms “biopsy tissue”, “patient sample”, “tumor sample”, and “cancer sample” refer to a sample of cells, tissue or fluid that is removed from a subject for the purpose of determining if the sample contains cancerous tissue, including cancer stem cells or for determining gene expression profile of that cancerous tissue. In some embodiment, biopsy tissue or fluid is obtained because a subject is suspected of having cancer. The biopsy tissue or fluid is then examined for the presence or absence of cancer, cancer stem cells, and/or cancer stem cell gene signature expression.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene can also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region can contain regulatory sequences such as promoters and enhancers that control or influence the transcription of the gene. The 3′ flanking region can contain sequences that direct the termination of transcription, post-transcriptional cleavage and polyadenylation.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs can also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene can be endogenous or exogenous to the organism, present integrated into a chromosome or present in a transfection vector that is not integrated into the genome. The expression of the gene is either completely or partially inhibited. RNAi can also be considered to inhibit the function of a target RNA; the function of the target RNA can be complete or partial.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region can be present in a cDNA, genomic DNA or RNA form. When present in a DNA form, the oligonucleotide or polynucleotide can be single-stranded (i.e., the sense strand) or double-stranded. Suitable control elements such as enhancers/promoters, splice junctions, polyadenylation signals, etc. can be placed in close proximity to the coding region of the gene if needed to permit proper initiation of transcription and/or correct processing of the primary RNA transcript. Alternatively, the coding region utilized in the expression vectors of the present invention can contain endogenous enhancers/promoters, splice junctions, intervening sequences, polyadenylation signals, etc. or a combination of both endogenous and exogenous control elements.

As used herein the term “portion” when in reference to a nucleotide sequence (as in “a portion of a given nucleotide sequence”) refers to fragments of that sequence. The fragments can range in size from four nucleotides to the entire nucleotide sequence minus one nucleotide (10 nucleotides, 20, 30, 40, 50, 100, 200, etc.).

The phrases “hybridizes”, “selectively hybridizes”, or “specifically hybridizes” refer to the binding or duplexing of a molecule only to a particular nucleotide sequence under stringent hybridization conditions when that sequence is present in a complex mixture (e.g., a library of DNAs or RNAs). See, e.g., Andersen (1998) Nucleic Acid Hybridization Springer-Verlag; Ross (ed. 1997) Nucleic Acid Hybridization Wiley.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times or 10 times background hybridization. Exemplary high stringency or stringent hybridization conditions include: 50% formamide, 5×SSC, and 1% SDS incubated at 42° C. or 5×SSC and 1% SDS incubated at 65° C., with a wash in 0.2×SSC and 0.1% SDS at 65° C. For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 sec, an annealing phase lasting 30-120 sec., and an extension phase of about 72° C. for 1-2 min.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide can be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide can be single-stranded), but can contain both the sense and anti-sense strands (i.e., the oligonucleotide or polynucleotide can be double-stranded).

“Amino acid sequence” and terms such as “polypeptide”, “protein”, or “peptide” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein can be produced by recombinant means or can be isolated from a naturally occurring source.

As used herein the term “portion” when in reference to a protein (as in “a portion of a given protein”) refers to fragments of that protein. The fragments can range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA can be cleaved with restriction enzymes prior to electrophoresis. Following electrophoresis, the DNA can be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies can be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and can include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 1.5-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28 S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” includes a specimen or culture obtained from any source, as well as biological and environmental samples. Biological samples can be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION

The present invention provides compositions and methods for treating, characterizing, and diagnosing cancer. In particular, the present invention provides gene expression profiles associated with solid tumor stem cells, as well as novel markers useful for the diagnosis, characterization, and treatment of solid tumor stem cells.

Like the tissues in which they originate, solid tumors consist of a heterogeneous population of cells. That the majority of these cells lack tumorigenicity suggested that the development and maintenance of solid tumors also relies on a small population of stem cells (i.e., tumorigenic cancer cells) with the capacity to proliferate and efficiently give rise both to additional tumor stem cells (self-renewal) and to the majority of more differentiated tumor cells that lack tumorigenic potential (i.e., non-tumorigenic cancer cells). The concept of cancer stem cells was first introduced soon after the discovery of hematopoietic stem cells (HSC) and was established experimentally in acute myelogenous leukemia (AML) (Park et al., 1971, J. Natl. Cancer Inst. 46:411-22; Lapidot et al., 1994, Nature 367:645-8; Bonnet & Dick, 1997, Nat. Med. 3:730-7; Hope et al., 2004, Nat. Immunol. 5:738-43). Stem cells from solid tumors have more recently been isolated based on their expression of a unique pattern of cell-surface receptors and on the assessment of their properties of self-renewal and proliferation in culture and in xenograft animal models. An ESA+ CD44+ CD24−/low Lineage-population greater than 50-fold enriched for the ability to form tumors relative to unfractionated tumor cells was discovered (Al-Hajj et al., 2003, Proc. Nat'l. Acad. Sci. 100:3983-8).

The ability to isolate tumorigenic cancer stem cells from the bulk of non-tumorigenic tumor cells has led to the identification of cancer stem cell markers, genes with differential expression in cancer stem cells compared to non-tumorigenic tumor cells or normal breast epithelium, using microarray analysis. The present invention employs the knowledge of these identified cancer stem cell markers to provide cancer stem cell gene signatures for the prognosis, diagnosis and treatment of cancer.

### Stem Cells and Solid Tumor Stem Cells

Common cancers arise in tissues that contain a large sub-population of proliferating cells that are responsible for replenishing the short-lived mature cells. In such organs, cell maturation is arranged in a hierarchy in which a rare population of stem cells give rise to the mature cells and perpetuate themselves through a process called self renewal (Akashi & Weissman, Developmental Biology of Hematopoiesis, Oxford Univ. Press, NY, 2001; Spangrude et al., 1988, Science 241:58-61; Baum et al., 1992, PNAS 89:2804-8; Morrison et al., 1995, PNAS 92:10302-6; Morrison et al., 1996, Immunity 5:207-16; Morrison et al., 1995, Annu. Rev. Cell Dev. Biol. 11:35-71; Morrison et al., 1997, Dev. 124:1929-39; Morrison & Weissman, 1994, Immunity 1:661; Morrison et al., 1997, Cell 88:287-98; Uchida et al., 2000, PNAS 97:14720-5; Morrison et al., 2000, Cell 101:499-510). Due to their rarity, stem cells should be isolated in order to study their biological, molecular, and biochemical properties. Although it is likely that they give rise to most tissues, stem cells have been rigorously identified and purified in only a few tissues. The stem cells that give rise to the lympho-hematopoietic system, called hematopoietic stem cells (HSCs), have been isolated from mice and humans and are the best characterized stem cells. The utility of tissue containing HSCs has been demonstrated in cancer therapy with their extensive use for bone marrow transplantation to regenerate the hematolymphoid system following myeloablative protocols (Baum et al., Bone Marrow Transplantation, Blackwell Scientific Publications, Boston, 1994). The prospective isolation of HSCs from patients can result in a population that is cancer free for autologous transplantation (Tricot et al., 1998, Blood 91:4489-95; Negrin et al., 2000, Biol Blood Marrow Transplantation 6:262-5; Michallet et al., 2000, Exp. Hematol. 28:858-70; Veona et al., 2002, Br. J. Haematol. 117:642-5; Barbui et al., 2002, Br. J. Haemat. 116:202-10).

Understanding the cellular biology of the tissues in which cancers arise, and specifically of the stem cells residing in those tissues, provides new insights into cancer biology. Several aspects of stem cell biology are relevant to cancer. First, both normal stem cells and cancer stem cells undergo self-renewal, and emerging evidence suggests that similar molecular mechanisms regulate self-renewal in normal stem cells and their malignant counterparts. Next, it is quite likely that mutations that lead to cancer accumulate in normal stem cells. Finally, it is likely that tumors contain a “cancer stem cell” population with indefinite proliferative potential that drives the growth and metastasis of tumors (Southam & Brunschwig, 1961, Cancer 14:971-78; Bruce & Gaag, 1963, Nature 199:79-80; Wodinsky et al., 1967, Cancer Chemother. Rep. 51:415-21; Bergsagel & Valeriote, 1968, Cancer Res. 28:2187-96; Park et al., 1971, J. Natl. Cancer Inst. 46:411-22; Hamburger & Salmon, 1977, Science 197:461-3; Lagasse & Weissman, 1994, J. Exp. Med. 179:1047-52; Reya et al., 2001, Nature 414:105-11; Al-Hajj et al., 2002, PNAS 100:3983).

HSCs are the most studied and best understood somatic stem cell population (Akashi & Weissman, Developmental Biology of Hematopoiesis, Oxford Univ. Press, NY, 2001). Hematopoiesis is a tightly regulated process in which a pool of hematopoietic stem cells eventually gives rise to the lymphohematopoietic system consisting of the formed blood elements, e.g., red blood cells, platelets, granulocytes, macrophages, and B- and T-lymphocytes. These cells are important for oxygenation, prevention of bleeding, immunity, and infections, respectively. In the adult, HSCs have two fundamental properties. First, HSCs need to self-renew in order to maintain the stem cell pool; the total number of HSCs is under strict genetic regulation (Morrison et al., 2002, J. Immunol. 168:635-42). Second, they must undergo differentiation to maintain a constant pool of mature cells in normal conditions, and to produce increased numbers of a particular lineage in response to stresses such as bleeding or infection.

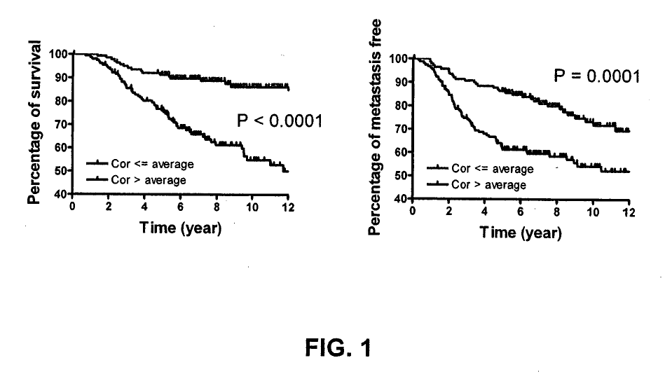

In the hematopoietic system, multipotent cells constitute 0.05% of mouse bone marrow cells and are heterogeneous with respect to their ability to self-renew. There are three different populations of multipotent cells: long-term self-renewing HSCs, short-term self-renewing HSCs, and multipotent progenitors without detectable self-renewal potential (Morrison & Weissman, 1994, Immunity 1:661; Christensen & Weissman, 2001, PNAS 98:14541-6). These populations form a hierarchy in which the long-term HSCs give rise to short-term HSCs, which in turn give rise to multipotent progenitors (FIG. 1 in Morrison & Weissman, 1994, Immunity 1:661). As HSCs mature from the long-term self-renewing pool to multipotent progenitors they become more mitotically active but lose the ability to self-renew. Only long-term HSCs can give rise to mature hematopoietic cells for the lifetime of the animal, while short-term HSCs and multipotent progenitors reconstitute lethally irradiated mice for less than eight weeks (Morrison & Weissman, 1994, Immunity 1:661).

Despite the fact that the phenotypic and functional properties of mouse and human HSCs have been extensively characterized (Baum et al., 1992, PNAS 89:2804-8), our understanding of the fundamental stem cell property, self-renewal, is minimal (Weissman, 2000, Science 287:1442; Osawa et al., 1996, Science 273:242-5; Reya et al., 2001, Nature 414:105-11). In most cases, HSCs differentiate when exposed to combinations of growth factors that can induce extensive proliferation in long-term cultures (Domen et al., 2000, J. Exp. Med. 192:1707-18). Although recent progress has been made in identifying culture conditions that maintain HSC activity in culture for a limited period of time (for example see Miller & Eaves, 1997, PNAS 94:13648-53), it has proven to be exceedingly difficult to identify tissue culture conditions that promote a significant and prolonged expansion of progenitors with transplantable HSC activity.

Maintenance of a tissue or a tumor is determined by a balance of proliferation and cell death (Hanahan & Weinberg, 2000, Cell 100:57-70). In a normal tissue, stem cell numbers are under tight genetic regulation resulting in maintenance a constant number of stem cells in the organ (Phillips et al., 1992, PNAS 89:11607-11; Muller-Sieburg et al., 2000, Blood 95:2446-8; Morrison et al., 2002, J. Immunol. 168:635-42). By contrast, cancer cells have escaped this homeostatic regulation and the number of cells within a tumor that have the ability to self renew is constantly expanding, resulting in the inevitable growth of the tumor. As would be expected, many of the mutations that drive tumor expansion regulate either cell proliferation or survival. For example, the prevention of apoptosis by enforced expression of the oncogene Bcl-2 promotes the development of lymphoma and also results in increased numbers of HSCs in vivo, suggesting that cell death plays a role in regulating the homeostasis of HSCs (Domen et al., 1998, Blood 91:2272-82; Domen et al., 2000, J. Exp. Med. 191:253-64). In fact, the progression to experimental acute myelogenous leukemia in mice requires at least 3, and likely 4 independent events to block the several intrinsically triggered and extrinsically induce programmed cell death pathways of myeloid cells (Traver et al., 1998, Immunity 9:47-57). Proto-oncogenes such as c-myb and c-myc that drive proliferation of tumor cells are also essential for HSCs development (Prochowinki & Kukowska, 1986, Nature 322:848-50; Clarke et al., 1988, Mol. Cellular. Biol. 8:884-92; Mucenski et al., 1991, Cell 65:677-89; Danish et al., 1992, Oncogene 7:901-7).

Since cancer cells and normal stem cells share the ability to self-renew, it is not surprising that a number of genes classically associated with cancer can also regulate normal stem cell development (reviewed in Reya et al., 2001, Nature 414:105-11 and Taipale & Beachy, 2001, Nature 411:349-54). In combination with other growth factors, Shh signaling has also been implicated in the regulation of self-renewal by the finding that cells highly enriched for human hematopoietic stem cells (CD34⁺Lin⁻CD38⁻) exhibited increased self-renewal in response to Shh stimulation in vitro (Bhardwaj et al., 2001, Nat. Immunol. 2:172-80). Several other genes related to oncogenesis have been shown to be important for stem cell function. For example, mice deficient for tal-1/SCL, which is involved in some cases of human acute leukemia, lack embryonic hematopoiesis (Shivdasani et al., 1995, Nature 373:432-4) suggesting that it is required for intrinsic or extrinsic events necessary to initiate hematopoiesis, for maintenance of the earliest definitive blood cells, or for the decision to form blood cells downstream of embryonic HSCs (Shivdasani et al., 1995, Nature 373:432-4; Porcher et al., 1996, Cell 86:47-57). Members of the Hox family have also been implicated in human leukemia. Enforced expression of HoxB4 can affect stem cell functions (Buske et al., 2002, Blood 100:862-681; Antonchuk & Humphries, 2002, Cell 109:39-45). One of the major targets of the p53 tumor suppressor gene is p21^{cip1}. Bone marrow from p21^{cip1} deficient mice has a reduced ability to serially reconstitute lethally irradiated recipients. Failure at serial transfer could result from exhaustion of the stem cell pool, loss of telomeres, or loss of transplantability (Cheng et al., 2000, Science 287:1804-8). In mice, bmi-1, a gene that cooperates with c-myc to induce lymphoma (van Lohuizen et al., 1991, Nature 353:353-55; van der Lugt et al., 1994, Genes & Dev. 8:757-69), is required for the maintenance of adult HSCs and leukemia cells. Thus, many genes involved in stem cell fate decisions are also involved in malignant transformation.

Two other signaling pathways implicated in oncogenesis in both mice and humans, the Wnt/β-catenin and Notch pathways, can play central roles in the self-renewal of both normal and cancer stem cells. The Notch family of receptors was first identified in Drosophila and has been implicated in development and differentiation (Artavanis-Tsakonas et al., 1999, Science 284:770-6). In C. elegans, Notch plays a role in germ cell self-renewal (Berry et al., 1997, Dev 124:925-36). In neural development transient Notch activation initiates an irreversible switch from neurogenesis to gliogenesis by embryonic neural crest stem cells (Morrison et al., 2000, Cell 101:499-510). Notch activation of HSCs in culture using either of the Notch ligands Jagged-1 or Delta transiently increased primitive progenitor activity that could be observed in vitro and in vivo, suggesting that Notch activation promotes either the maintenance of progenitor cell multipotentiality or HSC self-renewal (Shelly et al., 1999, J. Cell Biochem. 73:164-75; Varnum-Finney et al., Nat. Med. 6:1278-81). While the Notch pathway plays a central role in development and the mouse int-3 oncogene is a truncated Notch4 (Gallahan & Callahan, 1997, Oncogene 14:1883-90), the role for Notch in de novo human cancer is complex and less well understood. Various members of the Notch signaling pathway are expressed in cancers of epithelial origin and activation by Notch by chromosomal translocation is involved in some cases of leukemia (Ellisen et al., 1991, Cell 66:649-61; Zagouras et al., 1995, PNAS 92:6414; Liu et al., 1996, Genomics 31:58-64; Capobianco et al., 1997, Mol. Cell. Biol. 17:6265-73; Leethanakul et al., 2000, Oncogene 19:3220-4). Microarray analysis has shown that members of the Notch pathway are often over-expressed by tumor cells (Liu et al., 1996, Genomics 31:58-64; Leethanakul et al., 2000, Oncogene 19:3220-4). A truncated Notch4 mRNA is expressed by some breast cancer cell lines (Imatani & Callahan, 2000, Oncogene 19:223-31). Overexpression of Notch1 leads to growth arrest of a small cell lung cancer cell line, while inhibition of Notch1 signals can induce leukemia cell lines to undergo apoptosis (Shelly et al., 1999, J. Cell Biol. 73:164-75; Artavanis-Tsakonas, 1999, Science 284:770-6; Jehn et al., 1999, J. Immunol. 162:635-8). Work by Miele and colleagues showed that activation of Notch-1 signaling maintains the neoplastic phenotype in Ras-transformed human cells (Weizen et al., 2002, Nat. Med. 8:979-86). They also found that in de novo cancers, cells with an activating Ras mutation also demonstrated increased expression of Notch-1 and Notch-4.

Wnt/β-catenin signaling also plays a pivotal role in the self-renewal of normal stem cells and malignant transformation (Cadigan et al., 1997, Genes & Dev. 11:3286-305; Austin et al., 1997, Blood 89:3624-35; Spink et al., 2000, EMBO 19:2270-9). The Wnt pathway was first implicated in MMTV-induced breast cancer where in deregulated expression of Wnt-1 due to proviral insertion resulted in mammary tumors (Tsukamoto et al., 1988, Cell 55:619-25; Nusse et al., 1991, Cell 64:231). Subsequently, it has been shown that Wnt proteins play a central role in pattern formation. Wnt-1 belongs to large family of highly hydrophobic secreted proteins that function by binding to their cognate receptors, members of the Frizzled and low-density lipoprotein receptor-related protein families, resulting in activation of β-catenin (Cadigan & Nusse, 1997, Dev 11:3286-305; Leethanakul et al., 2000, Oncogene 19:3220-4; Reya et al., 2000, Immunity 13:15-24; Wu et al., 2000, Dev. 127:2773-84; Taiple & Beachy, 2001, Nature 411:349-54). In the absence of receptor activation, β-catenin is marked for degradation by a complex consisting of the Adenomatous Polyposis Coli (APC), Axin and glycogen synthase kinase-3β proteins (Austin et al., 1997, Blood 89:3624-35; van den Berg et al., 1998, Blood 92:3189-202; Gat et al., 1998, Cell 95:605-14; Chan et al., 1999, Nat. Genet. 21:410-3; Hedgepeth et al., 1999, Mol Cell Biol. 19:7147-57; Spink et al., 2000, EMBO 19:2270-9; Leethanakul et al., 2000, Oncogene 19:3220-4). Wnt proteins are expressed in the bone marrow, and activation of Wnt/β-catenin signaling by Wnt proteins in vitro or by expression of a constitutively active β-catenin expands the pool of early progenitor cells and enriched normal transplantable hematopoietic stem cells in tissue culture and in vivo (Austin et al., 1997, Blood 89:3624-35; van den Berg et al., 1998, Blood 92:3189-202; Reya et al., 2001, Nature 414:105-11). Inhibition of Wnt/β-catenin by ectopic expression of Axin, an inhibitor of β-catenin signaling, leads to inhibition of stem cell proliferation both in vitro and in vivo. Other studies suggest that the Wnt/β-catenin pathway mediates stem or progenitor cell self-renewal in other tissues (Gat et al., 1998, Cell 95:605-14; Korinek et al., 1998, Nat. Genet. 19:379-83; Zhu & Watt, 1999, Dev. 126:2285-98; Chan et al., 1999, Nat. Genet. 21:410-3). Higher levels of β-catenin are seen in keratinocytes with higher proliferative potential than those seen in keratinocytes with lower proliferative capacity (Gat et al., 1998, Cell 95:605-14; Chan et al., 1999, Nat. Genet. 21:410-3; Zhu & Watt, 1999, Dev. 126:2285-98). Like their normal hematopoietic stem cell counterparts, enforced expression of an activated β-catenin increased the ability of epidermal stem cells to self renew and decreased their ability to differentiate. Mice that fail to express TCF-4, one of the transcription factors that is activated when bound to β-catenin, soon exhaust their undifferentiated crypt epithelial progenitor cells, further suggesting that Wnt signaling is involved in the self renewal of epithelial stem cells (Korinek et al., 1998, Nat. Genet. 19:379-83; Taipale & Beachy, 2001, Nature 411:349-54).

Activation of β-catenin in colon cancer by inactivation of the protein degradation pathway, most frequently by mutation of APC, is common (Hedgepeth et al., 1999, Mol. Cell. Biol. 19:7147-57; Leethanakul et al., 2000, Oncogene 19:3220-4; Spink et al., 2000, EMBO 19:2270-9; Taipale & Beachy, 2001, Nature 411:349-54). Expression of certain Wnt genes is elevated in some other epithelial cancers suggesting that activation of β-catenin is secondary to ligand activation in such cancers (Nusse, 1992, J. Steroid Biochem. Mol. Biol. 43:9-12; Cadigan & Nusse, 1997, Genes & Dev. 11:3286-305; Kirkoshi et al., 2001, Int. J. Oncol. 19:997-1001; van de Wetering et al., 2002, Cell 111:241-50; Weeraratna et al., 2002, Cancer Cell 1:279-88; Saitoh et al., 2002, Int. J. Oncology 20:343-8; Saitoh et al., 2002, Int. J. Mol. Med. 9:515-9). There is evidence that constitutive activation of the Wnt/β-catenin pathway can confer a stem/progenitor cell phenotype to cancer cells. Inhibition of β-catenin/TCF-4 in a colon cancer cell line induced the expression of the cell cycle inhibitor p21^{cip-1} and induced the cells to stop proliferating and to acquire a more differentiated phenotype (van de Wetering et al., 2002, Cell 111:241-50). Enforced expression of the proto-oncogene c-myc, which is transcriptionally activated by β-catenin/TCF-4, inhibited the expression of p21^{cip-1} and allowed the colon cancer cells to proliferate when β-catenin/TCF-4 signaling was blocked, linking Wnt signaling to c-myc in the regulation of cell proliferation and differentiation. Although many studies have implicated the Wnt/β-catenin pathway in breast cancer, activating mutations of β-catenin are rare in this disease and no studies have definitively linked this pathway to human breast cancer (Candidus et al., 1996, Cancer Res. 56:49-52; Sorlie et al., 1998, Hum. Mutat. 12:215; Jonsson et al., 2000, Eur. J. Cancer 36:242-8; Schlosshauer et al., 2000, Cancinogenesis 21:1453-6; Lin et al., 2000, PNAS 97:4262-6; Wong et al., 2002, J. Pathol. 196:145-53).

The implication of roles for genes like Notch, Wnt, c-myc and Shh in the regulation of self-renewal of HSCs and perhaps of stem cells from multiple tissues suggests that there can be common self-renewal pathways in many types of normal somatic stem cells and cancer stem cells. It is important to identify the molecular mechanisms by which these pathways work and to determine whether the pathways interact to regulate the self-renewal of normal stem cells and cancer cells.

The Wnt pathway is involved in the self-renewal of normal stem cells and activating mutations of Wnt induce breast cancer in mice. This pathway plays a role in tumor formation by human breast cancer stem cells isolated from some patients. Furthermore, evidence suggests that the ability of different populations of breast cancer cells to form tumors differs. Interestingly, the expression of members of the Wnt/Frizzled/β-catenin pathway are heterogeneously expressed by different populations of cancer cells and expression of particular members of the pathway can correlate with the capacity to form tumors.

The different populations of cancer cells and tumor cells drive the proliferation of breast cancer cells. Activated β-catenin is seen in the cancer cells in a significant number of patients. The tumors that contain cancer cells with this pathway constitutively active behave differently than those without constitutively activated β-catenin.

### Solid Tumor Stem Cells Cancer Markers

The present invention provides markers whose expression is specifically altered in solid tumor stem cells (e.g. up regulated or down regulated). Such markers find use in the diagnosis and characterization and alteration (e.g., therapeutic targeting) of various cancers (e.g. breast cancer). Some cancer markers are provided below in Tables 9A-9N2. Cancer stem cell markers predictive of clinical outcome are provided in Table 10-17. While these tables provide gene names, it is noted that the present invention contemplates the use of both the nucleic acid sequences as well as the peptides encoded thereby, as well as fragments of the nucleic acid and peptides, in the therapeutic and diagnostic methods and compositions of the present invention.

### Solid Tumor Stem Cells Gene Signatures

The present invention provides the means and methods for classifying tumors based upon the profiling of solid tumor samples by comparing a gene expression profile of a cancer sample to a cancer stem cell gene signature. This invention identifies tumor stem cell gene signatures that are predictors of distant metastases and death. The microarray data of the present invention identifies cancer stem cell markers likely to play a role in breast cancer development, progression, and/or maintenance while also identifying cancer stem cell gene signatures useful in classifying breast tumors into low and high risk of, for example, metastasis and death. Classification based on the detection of differentially expressed polynucleotides and/or proteins that comprise a cancer gene profile when compared to a cancer stem cell gene signature can be used to predict clinical course, predict sensitivity to chemotherapeutic agents, guide selection of appropriate therapy, and monitor treatment response. Furthermore, following the development of therapeutics targeting such cancer stem cell markers, detection of cancer gene signatures described in detail below will allow the identification of patients likely to benefit from such therapeutics.

As described herein, the invention employs methods for clustering genes into gene expression profiles by determining their expression levels in two different cell or tissue samples. The invention further envisions using these gene profiles as compared to a cancer stem cell gene signature to predict clinical outcome including, for example, metastasis and death. The microarray data of the present invention identifies gene profiles comprising similarly and differentially expressed genes contained on the Affymetrix HG-U133 array between two tissue samples, one a test sample and one a reference sample, including between tumor stem cells and normal breast epithelium, non-tumorigenic tumor cells and normal breast epithelium, and tumor stem cells and non-tumorigenic tumor cells. These broad gene expression profiles can then be further refined, filtered, and subdivided into gene signatures based on various different criteria including, but not limited to, fold expression change, statistical analyses (e.g. t-test P value from multiple compared samples), biological function (e.g. cell cycle regulators, transcription factors, proteases, etc.), some therapeutic targets (e.g. genes encoding extracellular membrane associated proteins suitable for antibody based therapeutics), identified expression in additional patient samples, and ability to predict clinical outcome.

Thus certain embodiments of the present invention, the genes differentially expressed in tumor stem cells versus normal breast epithelium are subdivided into different cancer stem cell gene signatures based on their fold expression change. For example genes with between 2 to 2.5 fold elevated (or reduced, or both elevated and reduced) expression in tumor stem cells can comprise one tumor stem cell gene signature, genes with between 2.5 to 3 fold elevated (or reduced, or both) expression can comprise another tumor stem cell gene signature. Alternatively, all genes above a certain fold expression change are included in a tumor stem cell gene signature. For example, all genes with a 1 fold or more reduced (or elevated, or both) expression in tumor stem cells can comprise one tumor stem cell gene signature, all genes with a 2 fold or more reduced (or elevated, or both) expression in tumor stem cells can comprise another tumor stem cell gene signature, and so on. In some embodiments, the genes differentially expressed in tumor stem cells versus normal breast epithelium are filtered by using statistical analysis. For example, all genes with elevated (or reduced, or both) expression with a t-test P value across samples between 0.01 and 0.005 can comprise one tumor stem cell gene signature, all genes with elevated (or reduced, or both) expression with a t-test P value across samples of 0.005 and 0.001 can comprise another tumor stem cell gene signature, and so on. Furthermore, gene expression analysis of independent patient samples or different cell lines can be compared to any cancer stem cell gene signature generated as described above. A tumor stem cell gene signature can be modified, for example, by calculating individual phenotype association indices as described (Glinsky et al., 2004, Clin. Cancer Res. 10:2272) to increase or maintain the predictive power of a given tumor stem cell gene signature. In addition a tumor stem cell gene signature can be further narrowed or expanded gene by gene by excluding or including genes subjectively (e.g. inclusion of a some therapeutic target or exclusion of a gene included in another gene signatures).

In further embodiments, a broad gene expression profile such as those generated by the Affymetrix HG-U133 array analyses of the present invention can be further refined, filtered, or subdivided into gene signatures based on two or more different criteria. In some embodiments of the present invention the genes differentially expressed in tumor stem cells versus normal breast epithelium are subdivided into different tumor stem cell gene signatures based on their fold expression change as well as their biological function. For example, all genes involved in cell cycle regulation with between 3 to 3.5 fold elevated (or reduced, or both) expression in tumor stem cells versus normal breast epithelium can comprise one tumor stem cell gene signature, all genes involved in cell cycle regulation with between 3.5 to 4 fold elevated (or reduced, or both) expression can comprise another tumor stem cell gene signature, all genes encoding extracellular membrane associated proteins with 4 fold or more elevated (or reduced, or both) expression can comprise another tumor stem cell gene signature, all genes encoding extracellular membrane associated proteins with 5 fold or more elevated (or reduced, or both) expression can comprise yet another tumor stem cell gene signature. The generated cancer stem cell gene signatures are then compared against gene expression analysis from independent cancer patient populations (referred to as the patient datasets), including: 295 early breast cancer patients from the Netherlands Cancer Institute (NKI) (van de Vijver et al., 2002, N. Eng. J. Med. 347:1999), 286 lymph node negative breast cancer patients from the Erasmus Medical Center (GEO accession GSE2034) (Wang et al., 2005, Lancet 365:671), 159 breast cancer patients (GEO accession GSE1456) (Pawitan et al., 2005, Breast Cancer Res. 7:R953); 236 primary breast cancers (GEO accession GSE 3494) (Miller et al., 2005, PNAS 102:13550); and 189 invasive breast carcinomas (GEO accession GSE2990) (Sotiriou et al., 2006, Cancer Inst. 98: 262-72) as described in detailed in the Examples below.

In certain embodiments, the genes differentially expressed in breast tumor stem cells versus normal breast epithelium are divided into different tumor stem cell gene signatures based on their fold expression change and by statistical analysis. Specifically, in some embodiments the microarray analysis of the invention was used to identify genes with two-fold reduced and two-fold elevated expression in tumorigenic cells versus normal breast epithelium. This tumor stem cell gene signature was then further filtered by the P value of a t-test of 0.005 or 0.012 between the tumorigenic and normal breast epithelium samples to generate two cancer stem cell gene signatures (gene signatures 1 & 2) comprising 215 and 367 genes, respectively (Tables 1A-2A). Because there was not always a one-to-one correspondence between the generated gene signatures and the arrays used to analyze expression of the different patient dataset, a separate gene profile was generated as shown in Tables 1B-D; 2B-D below for each dataset without a complete gene signature.

TABLE 1A
Cancer Stem Cell Gene Signature 1

Elevated Expression
ANLN, AURKA, RRM2, IFI27, CDC2, Probe Set ID 228273, NUSAP1, CDCA1, SAMD9, KDELR2,
MELK, ECOP, GPR126, LOC652689 /// FAM72A /// LOC653594 /// LOC653820, STK39, LOC493869,
SKP2, FAM33A, ZC3HAV1L, CASC5, CEP170, TMEM106B, KDELR3, HN1, MATN2, DDEF1,
CEP350, IDH3A, MRPL15, EIF2S2, GPR180, STAT1, CHAC2, HS2ST1, MCAM, FLJ90709,
AMMECR1, STAMBPL1, CDKN2C, CPSF2, EPRS, C1orf25, NOL8, PSMD5, JUB, FLRT3, RPE,
IMPA2, GNPDA1, Probe Set ID 238191, TMF1, ANKRD25, HS2ST1, DNAPTP6, NUP37, TXNDC9,
UBE2V1 /// Kua-UEV, GTF2E1, C1orf107, MRPS17, ACP1, PDE8A, CDK8, HTLF, ATP13A3, LRP11,
RBM12B, PLP2, FAM82B, PREPL, SNX6, GEMIN6, SOAT1, INTS8, MAPK14, NUDT5, ATP2C1,
GBE1, C7orf36, TNKS, AGPS, COPB2, MGC12966, C16orf33, NCBP1, IARS, KIAA1600, CKAP5,
RPS6KC1, TRIM14, QSER1, E2F7, Probe Set ID 226347, INTS2, NUCKS1, AZI2, ABCA1, MGAT2,
C15orf23, DCUN1D4, ANKRD13C, SLC35B4, IKBKAP, JTV1, FAM45B /// FAM45A, KIAA0391,
NIPA2, C7orf30, NPAT, MRPS14, CSNK2A1, UMPS, WDR3, TMEM140, C11orf17 /// NUAK2,
ERGIC1, SLC12A8
Reduces Expression
LOC89944, C11orf32, SEC14L2, ARC, HSBP1, EPHA2, CUL5, TP53, ERN1, LOC285550, MLF1,
TPD52, SOSTDC1, SLC24A3, TMC4, LOC130576, FLJ37453, RAI2, PEBP1, SORBS2, Probe Set ID
228710, SERTAD1, WDR37, Probe Set ID 235247, CHN1, FLJ41603, ARID5A, CDKN1C, ROPN1,
DYNLRB2, GGT6, ARSD, JUND, Probe Set ID 238824, SLC24A3, SGK3, BNIPL, SOS2, TMEM101,
USP53, CLDN4, AXUD1, OVOL1, ETS2, FOLR1, LOH11CR2A, PER2, JUN, C1orf79, Probe Set ID
238725, Probe Set ID 230233, SHANK2, BCL2, ROPN1B, ETNK1, SATB1, RUNX1, H3F3B,
GOLGA8A, CIRBP, DSCR1, MAST4, Probe Set ID 242354, KIAA1324, AK5, KLHL21, ITPKC, Probe
Set ID 228049, SFPQ, CDC42EP5, CCL28, MAFF, ANPEP, STC2, CLIC6, FOSB, GADD45B, DMN,
RGC32, CX3CL1, IRX1, MUC15, ZDHHC2, Probe Set ID 229659, SCGB3A1, GABRP, CITED1,
BBOX1, GPM6B, CLDN8, KIT, SFRP1, Probe Set ID 242904, Probe Set ID 228854, LTF /// LOC643349,
KRT15, PTN, PIP

TABLE 1B
Cancer Gene Profile 1A Generated from the NKI Array

GBE1, ARSD, C1orf25, MATN2, BNIPL, RPS6KC1, DDEF1, MGAT2, E2F7, PLP2, LOC285550, IFI27,
ANKRD25, LOC130576, GEMIN6, ANPEP, GPM6B, KIAA0391, STC2, HN1, TP53, CDKN1C,
MGC12966, RRM2, C7orf36, PDE8A, DSCR1, TMC4, ABCA1, EPRS, MRPS17, NOL8, CLIC6, TMF1,
AXUD1, GABRP, KDELR2, ZDHHC2, FAM33A, MRPL15, SFPQ, CDKN2C, PER2, MAFF, HSBP1,
SFRP1, KRT15, HTLF, AK5, DMN, NCBP1, SERTAD1, JUND, ACP1, SOAT1, WDR3, UMPS,
SLC35B4, RAI2, IARS, CITED1, ATP2C1, TNKS, NUP37, EIF2S2, TXNDC9, NIPA2, FLRT3, TPD52,
KIT, PIP, HS2ST1, IDH3A, GNPDA1, MELK, MCAM, CHN1, CSNK2A1, AMMECR1, BBOX1,
ETNK1, C11orf32, ARC, MAPK14, NUSAP1, ITPKC, STK39, FLJ90709, SLC24A3, DNAPTP6,
MRPS14, CDK8, CLDN4, CUL5, SAMD9, RUNX1, STAT1, SEC14L2, KDELR3, ETS2, SOSTDC1,
BCL2, C7orf30, KLHL21, CDC2, SKP2, JTV1, NPAT, CX3CL1, ATP13A3, PSMD5, CIRBP, CDCA1,
GTF2E1, GADD45B, FOLR1, IMPA2, RPE, COPB2, IKBKAP, TRIM14, GPR126, CPSF2, SHANK2,
C16orf33, ERN1, SOS2, SATB1, JUN, H3F3B, RGC32, OVOL1, EPHA2, IRX1, FLJ41603, SNX6,
WDR37, LOH11CR2A, CLDN8, ANLN, FOSB, C15orf23

TABLE 1C
Cancer Gene Profile 1B Generated from the GSE2034 Array

GBE1, MLF1, MATN2, TMEM106B, RPS6KC1, MGAT2, PLP2, IFI27, ANKRD25, GEMIN6, ANPEP,
GPM6B, UBE2V1 /// Kua-UEV, KIAA0391, STC2, HN1, CDKN1C, RRM2, PDE8A, DSCR1, ABCA1,
PEBP1, PREPL, EPRS, MRPS17, NOL8, TMF1, GABRP, GOLGA8A, KDELR2, FAM82B, MRPL15,
SFPQ, CDKN2C, PER2, MAFF, HSBP1, SFRP1, KRT15, INTS8, KIAA1324, AK5, DMN, NCBP1,
JUND, ACP1, ROPN1B, SOAT1, WDR3, UMPS, RAI2, IARS, CITED1, ATP2C1, CKAP5, TNKS,
NUP37, MAST4, LOC89944, EIF2S2, TXNDC9, NIPA2, KIT, PIP, HS2ST1, IDH3A, LTF ///
LOC643349, GNPDA1, MELK, FAM45B /// FAM45A, MCAM, CHN1, CSNK2A1, BBOX1, C11orf32,
ARC, MAPK14, NUSAP1, ITPKC, STK39, SLC24A3, DNAPTP6, MRPS14, CDK8, CLDN4, STAT1,
SEC14L2, KDELR3, ETS2, SOSTDC1, BCL2, C11orf17 /// NUAK2, KLHL21, CEP350, DCUN1D4,
CDC2, SKP2, JTV1, NPAT, CX3CL1, ATP13A3, PSMD5, CEP170, CIRBP, GTF2E1, GADD45B,
C1orf107, FOLR1, IMPA2, COPB2, IKBKAP, TRIM14, GPR126, ARID5A, C16orf33, SATB1, JUN,
H3F3B, SLC24A3, RGC32, EPHA2, PTN, AURKA, LOH11CR2A, CLDN8, FOSB

TABLE 1D
Cancer Gene Profile 1C Generated from the GSE2990 Array

GBE1, MLF1, MATN2, TMEM106B, RPS6KC1, MGAT2, PLP2, IFI27, ANKRD25, GEMIN6, ANPEP,
GPM6B, UBE2V1 /// Kua-UEV, KIAA0391, STC2, HN1, CDKN1C, RRM2, PDE8A, DSCR1, ABCA1,
PEBP1, PREPL, EPRS, MRPS17, NOL8, TMF1, GABRP, GOLGA8A, KDELR2, FAM82B, MRPL15,
SFPQ, CDKN2C, PER2, MAFF, HSBP1, SFRP1, KRT15, INTS8, KIAA1324, AK5, DMN, NCBP1,
JUND, ACP1, ROPN1B, SOAT1, WDR3, UMPS, RAI2, IARS, CITED1, ATP2C1, CKAP5, TNKS,
NUP37, MAST4, LOC89944, EIF2S2, TXNDC9, NIPA2, KIT, PIP, HS2ST1, IDH3A, LTF ///
LOC643349, GNPDA1, MELK, FAM45B /// FAM45A, MCAM, CHN1, CSNK2A1, BBOX1, C11orf32,
ARC, MAPK14, NUSAP1, ITPKC, STK39, SLC24A3, DNAPTP6, MRPS14, CDK8, CLDN4, STAT1,
SEC14L2, KDELR3, ETS2, SOSTDC1, BCL2, C11orf17 /// NUAK2, KLHL21, CEP350, DCUN1D4,
CDC2, SKP2, JTV1, NPAT, CX3CL1, ATP13A3, PSMD5, CEP170, CIRBP, GTF2E1, GADD45B,
C1orf107, FOLR1, IMPA2, COPB2, IKBKAP, TRIM14, GPR126, ARID5A, C16orf33, SATB1, JUN,
H3F3B, SLC24A3, RGC32, EPHA2, PTN, AURKA, LOH11CR2A, CLDN8, FOSB

TABLE 2A
Cancer Stem Cell Gene Signature 2

Elevated Expression
ANLN, H19, DLG7, RRM2, IFI27, CDC2, Probe Set ID 228273, TOP2A, NUSAP1, CDCA1, CCNB1,
PLS3, SAMD9, KDELR2, HMMR, RACGAP1, MELK, ECOP, CEP55, CDCA7, LAMB1, GPR126,
PHACTR2, GINS1, LOC652689 /// FAM72A /// LOC653594 /// LOC653820, KIF11, SYT13, RAB31,
ECT2, DEPDC1, AURKA, CDC20, BUB1B, STK39, CDCA3, LOC493869, Probe Set ID 212993, SKP2,
FAM33A, ZC3HAV1L, RAD51AP1, CASC5, AMMECR1, CEP170, TMEM106B, ATP11A, KDELR3,
HN1, MATN2, DDEF1, UHRF1, CEP350, NUP205, CKAP2, ADCY7, CSE1L, CCNB2, IDH3A,
MRPL15, EIF2S2, GPR180, STAT1, DIAPH3, CHAC2, HS2ST1, ZWINT, MCAM, GAS2L3, FLJ90709,
STAMBPL1, CDKN2C, CPSF2, SLC25A13, PARP12, EPRS, ISG15, C1orf25, UBE2T, NOL8, PSMD5,
JUB, C14orf125, OIP5, PAK3 /// UBE2C, SGOL2, FLRT3, NHSL1, RPE, IMPA2, GNPDA1, MCART1,
CAMSAP1L1, HIST1H2BK, Probe Set ID 238191, TMF1, GMPS, ANKRD25, SEC23IP, FZD2,
DNAPTP6, NUP37, TXNDC9, IQGAP3, UBE2V1 /// Kua-UEV, GTF2E1, C1orf131, C1orf107, KIF15 ///
C7orf9, C9orf64, MRPS17, MTSS1, ACP1, KLHL12, HDHD1A, HK2, PTK2, WDR32, PDE8A, CDK8,
HTLF, NUCKS1, ATP13A3, LRP11, Probe Set ID 225917, PLSCR1, RBM12B, PLP2, FAM82B, PREPL,
SNX6, GEMIN6, SOAT1, KIAA0103, INTS8, MAPK14, CAPZA2, SKIV2L2, NUDT5, ATP2C1, GBE1,
C7orf36, CCDC99, TNKS, AGPS, RALA, SLC16A1, TBL1XR1, OSBPL11, COPB2, USP9X,
MGC12966, C16orf33, DHX29, NUP133, RAB23, POLE2, NCBP1, IARS, BRI3BP, TMPO, KIAA1600,
MBD4, MOBK1B, ORC5L, Probe Set ID 226348, CKAP5, ASCC3, TRRAP, RPS6KC1, Probe Set ID
229174, TRIM14, SLC38A6, QSER1, ATG3, ATP5J2, E2F7, BUD31, SAR1A, Probe Set ID 226347,
CDV3, Probe Set ID 228963, INTS2, PGM1, C10orf137, AZI2, SASS6, ABCA1, MGAT2, Probe Set ID
224778, C15orf23, DCUN1D4, ELP4, ANKRD13C, SLC35B4, SRP54, C12orf4, IKBKAP, JTV1,
FAM45B /// FAM45A, DLEU2 /// DLEU2L, NAV1, E2F8, KIAA0391, FADD, SECTM1, NIPA2,
C7orf30, MYCBP2, NPAT, MRPS14, CSNK2A1, UMPS, WDR3, MND1, TMEM140, SEC61G, RMI1,
C11orf17 /// NUAK2, UBLCP1, ERGIC1, SLC12A8
Reduced Expression
LOC89944, C11orf2, ACAA1, Probe Set ID 243791, CMTM7, FLJ10038, JUNB, C11orf32, SEC14L2,
ARC, HSBP1, TBC1D8, LOC283481, EPHA2, EIF4EBP3 /// MASK-BP3, CUL5, TP53, ERN1,
ANGPTL4, LOC285550, RND1, MGC39606 /// LOC644596, MLF1, TPD52, NDRG2, MGC7036,
SLC44A1, Probe Set ID AFFX-M27830, SOSTDC1, NFIB, TMC4, LOC130576, FLJ37453, RAI2, KLF9,
PEBP1, SORBS2, Probe Set ID 228710, SERTAD1, WDR37, HNMT, NEDD9, Probe Set ID 235247,
CHN1, FLJ41603, ARID5A, CDKN1C, MIA, ROPN1, PODXL, DYNLRB2, ERCC1, GGT6, CXorf10 ///
LOC648176, VSIG2, ARSD, JUND, Probe Set ID 238824, RUNX1, SLC24A3, SGK3, SOX10, BNIPL,
SOS2, CHKB /// CPT1B /// ARHGAP29, TMEM101, USP53, PCYT1B, FTO, CLDN4, RPL37, CCL28,
AXUD1, TTC18, OVOL1, ETS2, FOLR1, NGFRAP1L1, LOH11CR2A, PER2, JUN, RRAD, C1orf79,
Probe Set ID 238725, Probe Set ID 230233, SHANK2, BCL2, ROPN1B, ETNK1, Probe Set ID 230863,
SATB1, H3F3B, GOLGA8A, CIRBP, DSCR1, MAST4, Probe Set ID 242354, CD24, C1orf21, AK5,
KLHL21, ITPKC, Probe Set ID 228049, SFPQ, C4orf7, CDC42EP5, PI15, MAFF, GABARAPL1 ///
GABARAPL3, TF, ANPEP, STC2, CLIC6, FOSB, EMP1, GADD45B, DMN, OLFM4, RGC32, CX3CL1,
IRX1, KIAA1324, MUC15, ZDHHC2, Probe Set ID 229659, XIST, SCGB3A1, GABRP, CITED1,
BBOX1, GPM6B, CLDN8, TM4SF18, CXCL2, RGS2, KIT, SFRP1, ELF5, Probe Set ID 242904,
SCGB1D2, Probe Set ID 228854, LTF /// LOC643349, MGP, KRT15, PTN, PIP

TABLE 2B
Cancer Gene Profile 2A Generated from the NKI Array

GBE1, ARSD, DLG7, HK2, KIF11, C1orf25, OSBPL11, LAMB1, ECT2, MATN2, CXCL2, BNIPL,
RPS6KC1, HDHD1A, BUB1B, DDEF1, MGAT2, RPL37, E2F7, PLP2, LOC285550, IFI27, HNMT,
ANKRD25, LOC130576, GEMIN6, MBD4, ANPEP, GPM6B, KIAA0391, STC2, HN1, FTO, NUP205,
TP53, CDKN1C, MGC12966, TBC1D8, ELF5, RRM2, C7orf36, PDE8A, DSCR1, CCNB1, RRAD,
C14orf125, C4orf7, TMC4, ABCA1, DHX29, EPRS, MGC7036, MRPS17, SECTM1, NOL8, CLIC6,
TMF1, AXUD1, GABRP, MCART1, KDELR2, ZDHHC2, FAM33A, MRPL15, SFPQ, CDKN2C, PER2,
MAFF, MOBK1B, HSBP1, SFRP1, KRT15, C12orf4, HTLF, AK5, SLC25A13, DMN, NCBP1,
SERTAD1, JUND, ATP11A, ACP1, SOAT1, WDR3, UMPS, SLC35B4, USP9X, RAI2, NFIB, IARS,
CITED1, ATP2C1, PHACTR2, DIAPH3, PTK2, SEC61G, NHSL1, TNKS, NUP37, HMMR, RGS2,
POLE2, OIP5, PLS3, CKAP2, EIF2S2, TXNDC9, SLC16A1, NIPA2, FLRT3, TPD52, VSIG2, KIT, PIP,
KIAA0103, HS2ST1, HIST1H2BK, IDH3A, MTSS1, ERCC1, MYCBP2, RAD51AP1, GNPDA1, MELK,
TOP2A, MCAM, TRRAP, CHN1, CSNK2A1, LOC283481, RACGAP1, RND1, SEC23IP, BBOX1,
ETNK1, PODXL, FZD2, C11orf32, OLFM4, CSE1L, SCGB1D2, ARC, PCYT1B, MAPK14, NUSAP1,
C10orf137, ITPKC, ACAA1, STK39, FLJ90709, NAV1, C1orf21, DNAPTP6, SYT13, MRPS14, CCNB2,
CDK8, CLDN4, CUL5, SAMD9, CDCA7, KLHL12, TF, STAT1, ANGPTL4, JUNB, SEC14L2, KDELR3,
ETS2, SOSTDC1, CD24, BCL2, ELP4, C7orf30, ZWINT, PGM1, KLHL21, RAB23, NUP133, CDC2,
SKP2, JTV1, SKIV2L2, NPAT, CX3CL1, ATP13A3, PSMD5, CIRBP, SRP54, CDCA1, GTF2E1,
GADD45B, TMPO, CDCA3, FOLR1, IMPA2, CDC20, CCL28, RPE, CAPZA2, COPB2, IKBKAP, MIA,
ATP5J2, TRIM14, UHRF1, GPR126, C11orf2, PLSCR1, NGFRAP1L1, CPSF2, EMP1, SHANK2,
C16orf33, ERN1, SOS2, SLC38A6, RALA, FADD, GMPS, NDRG2, SATB1, JUN, H3F3B, RGC32,
SGOL2, FLJ10038, OVOL1, EPHA2, ORC5L, IRX1, FLJ41603, PI15, MGP, RAB31, ADCY7, SNX6,
WDR37, LOH11CR2A, CLDN8, ANLN, NEDD9, FOSB, C15orf23

TABLE 2C
Cancer Gene Profile 2B Generated from the GSE2034 Array

GBE1, DLG7, HK2, KIF11, MLF1, OSBPL11, LAMB1, ECT2, MATN2, CXCL2, ATG3, TMEM106B,
CHKB /// CPT1B /// ARHGAP29, RPS6KC1, HDHD1A, BUB1B, MGAT2, PLP2, IFI27, ANKRD25,
GEMIN6, MBD4, GINS1, ANPEP, GPM6B, UBE2V1 /// Kua-UEV, KIAA0391, STC2, HN1, FTO,
NUP205, CDKN1C, ELF5, RRM2, PDE8A, DSCR1, CCNB1, RRAD, ABCA1, PEBP1, DHX29, BUD31,
PREPL, EPRS, MRPS17, SECTM1, PARP12, NOL8, TMF1, GABRP, GOLGA8A, KDELR2, FAM82B,
SAR1A, MRPL15, SFPQ, CDKN2C, PER2, MAFF, MOBK1B, HSBP1, SFRP1, KRT15, CEP55, C12orf4,
Probe Set ID 212993, INTS8, EIF4EBP3 /// MASK-BP3, AK5, SLC25A13, DMN, NCBP1, JUND, ACP1,
ROPN1B, SOAT1, WDR3, UMPS, USP9X, RAI2, E2F8, IARS, CITED1, ATP2C1, CKAP5, PHACTR2,
PTK2, SEC61G, TNKS, NUP37, HMMR, RGS2, POLE2, MAST4, Probe Set ID AFFX-M27830, OIP5,
PLS3, CKAP2, LOC89944, EIF2S2, ISG15, TXNDC9, SLC16A1, NIPA2, CAMSAP1L1, KIT, PIP,
KIAA0103, HS2ST1, HIST1H2BK, IDH3A, MTSS1, MYCBP2, RAD51AP1, LTF /// LOC643349,
GNPDA1, MELK, TOP2A, WDR32, FAM45B /// FAM45A, MCAM, TRRAP, CHN1, CSNK2A1,
RACGAP1, NUCKS1, RND1, SEC23IP, BBOX1, PODXL, FZD2, C11orf32, OLFM4, CSE1L,
AMMECR1, SCGB1D2, ARC, MAPK14, NUSAP1, C10orf137, ITPKC, ACAA1, STK39, DNAPTP6,
MRPS14, CCNB2, CDK8, CLDN4, GABARAPL1 /// GABARAPL3, TF, STAT1, ANGPTL4, JUNB,
SEC14L2, KDELR3, ETS2, SOSTDC1, CD24, BCL2, ELP4, ZWINT, C11orf17 /// NUAK2, PGM1,
KLHL21, NUP133, CEP350, DCUN1D4, AURKA, RMI1, CDC2, SKP2, JTV1, SKIV2L2, NPAT,
CX3CL1, ATP13A3, PSMD5, CEP170, CIRBP, KIF15 /// C7orf9, SRP54, GTF2E1, GADD45B,
C1orf107, CCDC99, FOLR1, IMPA2, CDC20, PAK3 /// UBE2C, CAPZA2, COPB2, IKBKAP, MIA,
ATP5J2, TRIM14, GPR126, C11orf2, ASCC3, PLSCR1, ARID5A, EMP1, C16orf33, SLC38A6, RALA,
RUNX1, FADD, DLEU2 /// DLEU2L, GMPS, NDRG2, SATB1, JUN, H3F3B, SLC24A3, RGC32,
FLJ10038, EPHA2, ORC5L, PTN, MGP, RAB31, ADCY7, LOH11CR2A, CLDN8, NEDD9, SOX10,
FOSB

TABLE 2D
Cancer Gene Profile 2C Generated from the GSE2990 Array

GBE1, DLG7, HK2, KIF11, MLF1, OSBPL11, LAMB1, ECT2, MATN2, CXCL2, ATG3, TMEM106B,
CHKB /// CPT1B /// ARHGAP29, RPS6KC1, HDHD1A, BUB1B, MGAT2, PLP2, IFI27, ANKRD25,
GEMIN6, MBD4, GINS1, ANPEP, GPM6B, UBE2V1 /// Kua-UEV, KIAA0391, STC2, HN1, FTO,
NUP205, CDKN1C, ELF5, RRM2, PDE8A, DSCR1, CCNB1, RRAD, ABCA1, PEBP1, DHX29, BUD31,
PREPL, EPRS, MRPS17, SECTM1, PARP12, NOL8, TMF1, GABRP, GOLGA8A, KDELR2, FAM82B,
SAR1A, MRPL15, SFPQ, CDKN2C, PER2, MAFF, MOBK1B, HSBP1, SERP1, KRT15, CEP55, C12orf4,
Probe Set ID 212993, INTS8, EIF4EBP3 /// MASK-BP3, AK5, SLC25A13, DMN, NCBP1, JUND, ACP1,
ROPN1B, SOAT1, WDR3, UMPS, USP9X, RAI2, E2F8, IARS, CITED1, ATP2C1, CKAP5, PHACTR2,
PTK2, SEC61G, TNKS, NUP37, HMMR, RGS2, POLE2, MAST4, Probe Set ID AFFX-M27830, OIP5,
PLS3, CKAP2, LOC89944, EIF2S2, ISG15, TXNDC9, SLC16A1, NIPA2, CAMSAP1L1, KIT, PIP,
KIAA0103, HS2ST1, HIST1H2BK, IDH3A, MTSS1, MYCBP2, RAD51AP1, LTF /// LOC643349,
GNPDA1, MELK, TOP2A, WDR32, FAM45B /// FAM45A, MCAM, TRRAP, CHN1, CSNK2A1,
RACGAP1, NUCKS1, RND1, SEC23IP, BBOX1, PODXL, FZD2, C11orf32, OLFM4, CSE1L,
AMMECR1, SCGB1D2, ARC, MAPK14, NUSAP1, C10orf137, ITPKC, ACAA1, STK39, DNAPTP6,
MRPS14, CCNB2, CDK8, CLDN4, GABARAPL1 /// GABARAPL3, TF, STAT1, ANGPTL4, JUNB,
SEC14L2, KDELR3, ETS2, SOSTDC1, CD24, BCL2, ELP4, ZWINT, C11orf17 /// NUAK2, PGM1,
KLHL21, NUP133, CEP350, DCUN1D4, AURKA, RMI1, CDC2, SKP2, JTV1, SKIV2L2, NPAT,
CX3CL1, ATP13A3, PSMD5, CEP170, CIRBP, KIF15 /// C7orf9, SRP54, GTF2E1, GADD45B,
C1orf107, CCDC99, FOLR1, IMPA2, CDC20, PAK3 /// UBE2C, CAPZA2, COPB2, IKBKAP, MIA,
ATP5J2, TRIM14, GPR126, C11orf2, ASCC3, PLSCR1, ARID5A, EMP1, C16orf33, SLC38A6, RALA,
RUNX1, FADD, DLEU2 /// DLEU2L, GMPS, NDRG2, SATB1, JUN, H3F3B, SLC24A3, RGC32,
FLJ10038, EPHA2, ORC5L, PTN, MGP, RAB31, ADCY7, LOH11CR2A, CLDN8, NEDD9, SOX10,
FOSB

In certain embodiments, the genes differentially expressed in colon tumor stem cells versus nontumorigenic colon cells are divided into different tumor stem cell gene signatures based on the fold expression change. Specifically, in some embodiments, the microarray analysis of the invention was used to identify genes with three-fold reduced and three-fold elevated expression in tumorigenic colon cells versus non-tumorigenic colon cells. This tumor stem cell gene signature was then further filtered by the P value of a t-test of 0.04 between the tumorigenic and non-tumorigenic samples to generate cancer stem cell gene signature 3 comprising 315 genes (Tables 3A). Because there was not a one-to-one correspondence between the generated gene signatures and the arrays used to analyze expression of the different patient datasets, a separate gene profile was generated as shown in Tables 3B-E below for each dataset without a complete gene signature.

TABLE 3A
Cancer Stem Cell Gene Signature 3

Elevated Expression
Probe Set ID 225716, BAG4, C6orf211, MAD2L1, FAM29A, USP38, GIPC2, ALDH6A1, PSIP1,
SLC39A8, RB1, ANP32E, Probe Set ID 228956, ATF1, RPE, PBK, CDC23, AGL, FAM98B, SPRED1,
ALS2CR13, HS2ST1, CMAS, SPFH2, ACTR6, ELK3, Probe Set ID 212698, XK, SPAST, C1orf58,
XPNPEP3, NAT12, TIFA, C3orf58, C21orf45, PAICS, AGPS, UBE2V2, KCTD20, MGC34646, TXNDC,
SMC1A, FBXO3, PPP1R2, SYBL1, IMPAD1, HOOK1, ARFIP1, TMED7, RNF138, YEATS4, CHML,
NXT2, MYCBP, USP1, LOC203427, SLC25A32, OSBPL8, TFB2M, Probe Set ID 212920, AADACL1,
CEP55, PRPS2, RPA1, GFM1, Probe Set ID 213750, MLSTD2, SEH1L, CGI-115, SLC35A5, LRRC40,
HIPK2, C14orf126, CBX1, HAT1, EGLN1, SLC39A14, PSAT1, Probe Set ID 236548, ISOC1, PRKDC,
G3BP2, TRAPPC6B, KIAA1430, METTL7A, ACBD5, GJB2, TGFBR1, AASDHPPT, FLJ14803, PCGF5,
COX11, Probe Set ID 227027, Probe Set ID 213549, NUDT15, MCFD2, CKAP2, GLCE, TFAM, Probe
Set ID 225179, SLC39A6, TMEM19, PPA2, TMOD3, LARP4, POLE3, RNF6, PGAP1, COX18,
SLC44A3, ALG14, ANKRD28, FLJ21908, BUB1, NAT13, Probe Set ID 234986, Probe Set ID 226336,
ZNF367, NUP98, SMARCAS, COX15, FBXL5, KIAA1458, Probe Set ID 225426, PRKAR1A, NEK4,
YES1, C10orf22, CCNB1, EIF3S1, TMEM135, C13orf7, PRTFDC1, LANCL1, PHF6, VPS24, HNRPH2,
FLJ11184, LOC203411, RAB21, MRPL19, PIGW, C17orf25, HBLD2, HIBADH, Probe Set ID 226662,
IER3IP1, SLC30A1, KIAA1815, C1orf80, DCTN4, RBM12, PGM2, FYTTD1, HMMR, Probe Set ID
227200, SHOC2, SH3BGRL, Probe Set ID 229173, DOCK9, HDHD2, CMPK, TCEA1, SEL1L, CHAC2,
KIAA0241, MRPL13, PKP2, MED4, UNG, TRIM14, CAB39, PDCD2, MRPS28, TSN, KPNA3, HNMT,
AGPAT5, SYPL1, KIAA1737, RRN3, PDSS2, MANEA, GTF2A1, TMEM33, Probe Set ID 213786,
CETN3, LOC221710, PIGY, WDR51B, FZD5, COMMD8, CPNE3, Probe Set ID 202377, ZNF770,
VPS26A, ZNRF3, SPFH1, CCDC6, LYCAT, DCK, NEBL, NT5E, PRKACB, RNF20, TMEM38B, IL6ST,
CDCA7L, BMPR2, ZAK, CHEK1, DKFZP686E2158, CDV3, STCH, HMGCR, SDHB, PANK1, RAP2A,
ELOVL6, SUMF1, Probe Set ID 226886, ECT2, RKHD2, APPL, SRD5A1, DLG7, NUP37, TLK1, CNIH,
C14orf130, Probe Set ID 224582, RNF141, EVA1, RP6-213H19.1, PSMA3, Probe Set ID 226251,
TXNDC9, FUNDC1, RCOR1, MRPS23, FLJ11506, ZFAND1, TMEM30A, DKC1, PRDX3, SLC6A6,
CCND2, TSPYL1, GUF1, MTMR2, DNAJC10, HMG20A, FZD6, SEMA3C, GCH1, KLHL15, RSC1A1,
HNF4G, UBE2E3, UCHL5, PTPN11, ATP13A3, ANLN, DEK, RP2, AEBP2, VPS4B, DSCR2, YWHAQ,
RACGAP1, Probe Set ID 224644, SLBP, FBXO45, CCDC117, LRRC57, GRPEL2, ZMPSTE24, KIF21A,
KIAA1826, KCTD3, SURF4, UBE2Q2, CPOX, GNS, GALNT7, RRM1, EPS15, PGRMC2, KBTBD6,
C12orf29, SOCS4, CCDC5, PTGER4, C5orf22, C1orf19, TSPAN12, PPIL1, GOLPH2, MAN1A1,
NHSL1, C16orf63
Reduced Expression
NPIP, NKTR, SCARNA2, MAPK8IP3, LOC338758, LOC339047, Probe Set ID 1558515, RASEF,
LOC152485, RNPC3, ATHL1, LOC645352, KIAA1641, LOC646769

TABLE 3B
Cancer Gene Profile 3A Generated from the NKI Array

EVA1, KCTD3, DLG7, YWHAQ, RRN3, DOCK9, ALDH6A1, PAICS, PGM2, ECT2, TXNDC, GTF2A1,
KIAA1430, POLE3, FLJ11506, FBXO45, CAB39, TFAM, TRAPPC6B, MTMR2, C21orf45, SLC39A14,
NT5E, PRKACB, C13orf7, MED4, DNAJC10, SLC39A8, MCFD2, RKHD2, TMEM38B, HBLD2,
SLC6A6, KPNA3, C17orf25, GCH1, TSN, ARFIP1, CCNB1, UBE2V2, RAB21, GOLPH2, MRPS28,
GIPC2, KIAA1826, XK, GJB2, PANK1, TFB2M, SURF4, SEMA3C, PPIL1, COX11, ELK3, VPS4B,
RB1, MANEA, LOC221710, RNF141, DSCR2, HOOK1, SMARCA5, RNF138, DEK, EIF3S1,
AASDHPPT, SLC35A5, HNRPH2, RAP2A, GNS, NXT2, RNF20, DCK, YEATS4, RRM1, KIF21A,
FBXL5, CGI-115, PTPN11, SLBP, UCHL5, NHSL1, RBM12, PSAT1, NUP37, HMMR, BUB1, CKAP2,
AGPS, PCGF5, RPA1, RP2, TCEA1, UNG, TGFBR1, TXNDC9, KIAA1458, TMEM19, LOC203411,
MAD2L1, SPAST, LYCAT, NKTR, RSC1A1, HS2ST1, SPRED1, CPNE3, HNF4G, TLK1, MRPL19,
IL6ST, SPFH2, HAT1, PHF6, PRPS2, CCND2, CHEK1, IER3IP1, SLC30A1, FLJ11184, RACGAP1,
PRKAR1A, YES1, WDR51B, CMAS, CPOX, AEBP2, CDC23, EPS15, C1orf19, SYBL1, KIAA1737,
FBXO3, MGC34646, GFM1, AADACL1, STCH, ISOC1, PSMA3, SEL1L, GRPEL2, NUDT15,
SH3BGRL, GALNT7, GLCE, PTGER4, BAG4, RNF6, CNIH, FYTTD1, PDCD2, FLJ14803, SDHB,
HMGCR, MAPK8IP3, TMED7, ATP13A3, PRTFDC1, PBK, PRKDC, KBTBD6, TMEM30A, USP1,
LOC339047, AGL, DCTN4, SPFH1, CHML, SOCS4, DKC1, COMMD8, ZMPSTE24, NPIP, CBX1,
FAM29A, MYCBP, UBE2E3, RPE, ATF1, RP6-213H19.1, NUP98, PGRMC2, BMPR2, TRIM14,
RCOR1, CETN3, ZAK, MRPL13, MAN1A1, HMG20A, PSIP1, SRD5A1, PPP1R2, PKP2, LOC338758,
C14orf130, CCDC6, C10orf22, FZD6, LANCL1, CCDC5, APPL, TMEM33, NEBL, G3BP2, SHOC2,
PRDX3, FZD5, NEK4, ANLN, OSBPL8, COX15

TABLE 3C
Cancer Gene Profile 3B Generated from the GSE2034 Array

EVA1, KCTD3, DLG7, YWHAQ, RRN3, DOCK9, ALDH6A1, PAICS, KIAA1641, Probe Set ID 213549,
ECT2, TXNDC, METTL7A, HNMT, POLE3, FLJ11506, SYPL1, VPS26A, CAB39, TFAM, ANP32E,
NAT13, ELOVL6, MTMR2, SLC39A14, NT5E, PRKACB, AGPAT5, C13orf7, VPS24, MED4,
SLC39A8, MCFD2, RKHD2, TMEM38B, HBLD2, KPNA3, C17orf25, GCH1, TSN, ARFIP1, CCNB1,
UBE2V2, RAB21, GOLPH2, MRPS28, SLC25A32, SEH1L, XK, SMC1A, ACTR6, TFB2M, TSPAN12,
SEMA3C, COX11, ELK3, VPS4B, RB1, DSCR2, ATHL1, Probe Set ID 213786, SMARCA5, RNF138,
DEK, C12orf29, EIF3S1, AASDHPPT, SLC39A6, SLC35A5, HNRPH2, CEP55, GNS, NXT2, DCK,
YEATS4, RRM1, FBXL5, CGI-115, PTPN11, SLBP, UCHL5, LRRC40, RBM12, NUP37, HMMR,
BUB1, CKAP2, FLJ21908, PDSS2, RPA1, TCEA1, UNG, TXNDC9, MAD2L1, SPAST, NXTR, CDV3,
KIAA0241, HS2ST1, C5orf22, CPNE3, LARP4, Probe Set ID 213750, TLK1, MRPL19, IL6ST, SPFH2,
HAT1, PRPS2, CCND2, CHEK1, TSPYL1, Probe Set ID 212698, SLC30A1, FLJ11184, RACGAP1,
PRKAR1A, YES1, CMAS, CPOX, CDC23, EPS15, SYBL1, FBXO3, STCH, ISOC1, PSMA3, SEL1L,
NUDT15, Probe Set ID 212920, SH3BGRL, TMEM135, GALNT7, GLCE, PTGER4, RNF6, CNIH,
C1orf80, PDCD2, CMPK, ZFAND1, SDHB, HMGCR, MAPK8IP3, TMED7, ATP13A3, PBK, Probe Set
ID 202377, PRKDC, TMEM30A, USP1, LOC339047, AGL, SPFH1, DKC1, COMMD8, ZMPSTE24,
NPIP, CBX1, MYCBP, UBE2E3, ATF1, C6orf211, RP6-213H19.1, NUP98, PGRMC2, TRIM14, RCOR1,
CETN3, MRPL13, MAN1A1, HMG20A, PSIP1, SRD5A1, PPP1R2, PKP2, C14orf130, C10orf22, FZD6,
LANCL1, APPL, TMEM33, NEBL, G3BP2, SHOC2, PRDX3, FZD5, NEK4, OSBPL8, COX15

TABLE 3D
Cancer Gene Profile 3C Generated from the GSE2990 Array

EVA1, KCTD3, DLG7, YWHAQ, RRN3, DOCK9, ALDH6A1, PAICS, KIAA1641, Probe Set ID 213549,
ECT2, TXNDC, METTL7A, HNMT, POLE3, FLJ11506, SYPL1, VPS26A, CAB39, TFAM, ANP32E,
NAT13, ELOVL6, MTMR2, SLC39A14, NT5E, PRKACB, AGPAT5, C13orf7, VPS24, MED4,
SLC39A8, MCFD2, RKHD2, TMEM38B, HBLD2, KPNA3, C17orf25, GCH1, TSN, ARFIP1, CCNB1,
UBE2V2, RAB21, GOLPH2, MRPS28, GIPC2, SLC25A32, SEH1L, XK, SMC1A, ACTR6, TFB2M,
TSPAN12, SEMA3C, COX11, ELK3, VPS4B, RB1, DSCR2, ATHL1, Probe Set ID 213786, SMARCA5,
RNF138, DEK, C12orf29, EIF3S1, AASDHPPT, SLC39A6, SLC35A5, HNRPH2, CEP55, GNS, NXT2,
DCK, YEATS4, RRM1, FBXL5, CGI-115, PTPN11, SLBP, UCHL5, LRRC40, RBM12, NUP37, HMMR,
BUB1, CKAP2, FLJ21908, PDSS2, RPA1, RP2, TCEA1, UNG, TXNDC9, MAD2L1, SPAST, NKTR,
CDV3, KIAA0241, HS2ST1, C5orf22, CPNE3, LARP4, Probe Set ID 213750, TLK1, MRPL19, IL6ST,
SPFH2, HAT1, PRPS2, CCND2, CHEK1, TSPYL1, Probe Set ID 212698, SLC30A1, FLJ11184,
RACGAP1, PRKAR1A, YES1, CMAS, CPOX, CDC23, EPS15, SYBL1, PGAP1, FBXO3, STCH, ISOC1,
PSMA3, SEL1L, NUDT15, Probe Set ID 212920, SH3BGRL, TMEM135, GALNT7, GLCE, PTGER4,
RNF6, CNIH, C1orf80, PDCD2, CMPK, ZFAND1, SDHB, HMGCR, MAPK8IP3, TMED7, ATP13A3,
PBK, Probe Set ID 202377, PRKDC, TMEM30A, USP1, LOC339047, AGL, SPFH1, DKC1, COMMD8,
ZMPSTE24, NPIP, CBX1, MYCBP, UBE2E3, ATF1, C6orf211, RP6-213H19.1, NUP98, PGRMC2,
TRIM14, RCOR1, CETN3, MRPL13, MAN1A1, HMG20A, PSIP1, SRD5A1, PPP1R2, PKP2, C14orf130,
C10orf22, FZD6, LANCL1, APPL, TMEM33, NEBL, G3BP2, SHOC2, PRDX3, FZD5, NEK4, OSBPL8,
COX15

TABLE 3E
Cancer Gene Profile 3D Generated from the GSE1456 and 3494 Arrays

EVA1, KCTD3, DLG7, YWHAQ, RRN3, DOCK9, ALDH6A1, PAICS, KIAA1641, Probe Set ID 213549,
PGM2, C1orf58, ECT2, TXNDC, EGLN1, METTL7A, GTF2A1, KIAA1430, HNMT, POLE3, FLJ11506,
SYPL1, FBXO45, VPS26A, CAB39, ALS2CR13, TFAM, HDHD2, TRAPPC6B, KLHL15, LRRC57,
ANP32E, NAT13, ELOVL6, MTMR2, C21orf45, SLC39A14, NT5E, PRKACB, AGPAT5, C13orf7,
VPS24, MED4, DNAJC10, SLC39A8, NAT12, MCFD2, RKHD2, TMEM38B, HBLD2, SLC6A6,
KPNA3, C17orf25, RNPC3, GCH1, TSN, ARFIP1, CCNB1, UBE2V2, LOC152485, UBE2Q2, RAB21,
GOLPH2, Probe Set ID 224582, PIGY, MRPS28, TMOD3, GIPC2, KIAA1826, SLC25A32, SEH1L,
CCDC117, XK, Probe Set ID 226662, GJB2, SMC1A, ACTR6, SLC44A3, PANK1, TFB2M, SURF4,
TSPAN12, SEMA3C, PPIL1, COX11, ELK3, VPS4B, RB1, MANEA, LOC221710, RNF141, USP38,
HIPK2, Probe Set ID 227027, DSCR2, ATHL1, HOOK1, Probe Set ID 213786, SMARCA5, ZNF770,
RNF138, DEK, C12orf29, EIF3S1, AASDHPPT, SLC39A6, SLC35A5, HNRPH2, CEP55, RAP2A,
XPNPEP3, GNS, NXT2, RNF20, DCK, YEATS4, RRM1, COX18, Probe Set ID 225426, KIF21A,
FBXL5, CGI-115, ZNRF3, PTPN11, SLBP, UCHL5, LRRC40, NHSL1, RBM12, PSAT1, NUP37,
HMMR, BUB1, CKAP2, FLJ21908, LOC645352, AGPS, TIFA, PCGF5, PDSS2, RPA1, RP2, TCEA1,
UNG, TGFBR1, TXNDC9, KIAA1458, TMEM19, LOC203411, MAD2L1, Probe Set ID 225179, SPAST,
LYCAT, NKTR, DKFZP686E2158, CDV3, RSC1A1, KIAA0241, HS2ST1, SPRED1, FUNDC1, Probe
Set ID 226336, C16orf63, C5orf22, CPNE3, Probe Set ID 236548, LARP4, KGTD20, FAM98B, HNF4G,
Probe Set ID 213750, Probe Set ID 229173, MLSTD2, CDCA7L, TLK1, MRPL19, C3orf58, IL6ST,
SPFH2, HAT1, PHF6, KIAA1815, Probe Set ID 224644, PRPS2, CCND2, CHEK1, TSPYL1, Probe Set ID
212698, IER3IP1, SLC30A1, FLJ11184, RACGAP1, PRKAR1A, YES1, WDR51B, CMAS, CPOX,
SCARNA2, AEBP2, CDC23, EPS15, C1orf19, SYBL1, KIAA1737, MRPS23, PGAP1, FBXO3,
MGC34646, Probe Set ID 226251, Probe Set ID 228956, GFM1, HIBADH, AADACL1, IMPAD1, STCH,
ISOC1, PSMA3, Probe Set ID 225716, SEL1L, Probe Set ID 227200, GRPEL2, NUDT15, Probe Set ID
212920, SH3BGRL, GUF1, TMEM135, GALNT7, GLCE, PTGER4, BAG4, RNF6, CNIH, FYTTD1,
C1orf80, PDCD2, FLJ14803, CMPK, ZFAND1, SDHB, HMGCR, MAPK8IP3, LOC646769, TMED7,
ATP13A3, PRTFDC1, PBK, Probe Set ID 202377, CHAC2, PRKDC, KBTBD6, TMEM30A, USP1,
LOC339047, AGL, Probe Set ID 234986, DCTN4, SPFH1, CHML, SOCS4, DKC1, ANKRD28,
COMMD8, ZMPSTE24, NPIP, CBX1, FAM29A, MYCBP, UBE2E3, C14orf126, RPE, ATF1, C6orf211,
RP6-213H19.1, NUP98, PGRMC2, ACBD5, BMPR2, TRIM14, RCOR1, CETN3, ZAK, MRPL13,
MAN1A1, HMG20A, PSIP1, SRD5A1, PPP1R2, PKP2, LOC338758, C14orf130, CCDC6, C10orf22,
FZD6, LANCL1, CCDC5, ZNF367, APPL, TMEM33, NEBL, G3BP2, SUMF1, SHOC2, PRDX3, FZD5,
NEK4, ANLN, OSBPL8, COX15, Probe Set ID 226886

In certain embodiments, the cancer gene signatures are identified by combining gene signatures. In some embodiments, the 186 gene cancer gene signature 3 from U.S. patent application Ser. No. 11/451,773, herein incorporated by reference, Table 3A was combined with the 367 gene cancer gene signature 2 in Table 2A above. The raw data image data of the Affymetrix array was processed using two different methods (see Table 34, file “C4_{—}6_{—}9_{—}3.txt” submitted on compact disc and incorporated by reference). In the 186 gene signature was processed using a from method developed by Kerby Shedden from University of Michigan: To obtain an expression measure for a given probe set, the mismatch hybridization values were subtracted from the perfect match values, and the average of the middle 50% of these differences was used as the expression measure for the probe set. A quantile normalization procedure was then applied to adjust for differences in the probe intensity distribution across different chips. Specifically, we applied a monotone linear spline to each chip that mapped quantiles 0.01 up to 0.99 (in increments of 0.01) exactly to the corresponding quantiles of a standard chip. The transform log 2 (200+max(X;0)) was then applied. The 367 gene cancer gene signature was processed using a method called RMA (robust multiarray average), for detail algorithm, see reference: Bolstad B. M., Irizarry R. A., Astrand M., and Speed, T. P. (2003), A Comparison of Normalization Methods for High Density Oligonucleotide Array Data Based on Bias and Variance. Bioinformatics 19(2):185-193).

These combined two signatures resulted in a 520 gene list.

520 GENE LIST

GBE1, ERGIC1, DNMT3A, LDHA, ARSD, DLG7, C4orf32, HK2, MLF1, KIF11, CCNB1, C1orf25,
ZC3HAV1L, OSBPL11, SLC12A8, LAMB1, MND1, ECT2, DBR1, KIAA1217, MATN2, ALKBH1,
METTL7A, ATG3, CXCL2, TMEM106B, FLJ37953, BNIPL, Probe Set ID 226191, CHKB /// CPT1B ///
ARHGAP29, RPS6KC1, Probe Set ID 228963, HDHD1A, BUB1B, DDEF1, MGAT2, RPL37, ARSD ///
DPH5, E2F7, CITED4, NUCKS1, PLP2, C1orf131, VIL2, GPM6B, LOC285550, IFI27, HNMT,
ANKRD25, LOC130576, C10orf7, WFDC2, GEMIN6, MBD4, ALG2, GINS1, Probe Set ID 229659,
ANPEP, FLNB, C10orf9, GPM6B, UBE2V1 /// Kua-UEV, KIAA0391, STC2, HN1, C17orf71, FTO,
CCDC43, XIST, JUB, SSR1, NUP205, TP53, CDKN1C, MGC12966, C7orf36, ELF5, TBC1D8, RRM2,
C1orf121, PDE8A, DSCR1, CCNB1, RRAD, CASP8, ZYG11B, C14orf125, C4orf7, TMC4, C9orf64,
KIAA1600, H19, Probe Set ID AFFX-M27830_5, Probe Set ID 242904, BMSC-MCP, LARS, ABCA1,
PEBP1, STAMBPL1, DHX29, BUD31, PREPL, EPRS, PNKD, MGC7036, MRPS17, SECTM1, SWAP70,
C5orf21, PLAA, PARP12, Probe Set ID 219216, NOL8, RAB31, CLIC6, TMF1, AXUD1, GABRP,
CX3CL1, GOLGA8A, MCART1, KDELR2, THEM2, C1orf163, FAM82B, ZDHHC2, Probe Set ID
226347, FAM33A, MRPL15, SAR1A, TMEM140, BRI3BP, SFPQ, ARSD /// DPH5, CDKN2C, PER2,
MAFF, UBE2F, MOBK1B, UBE2T, GAPDH, HSBP1, KLF9, SFRP1, KRT15, CEP55, GTF3C3, C12orf4,
KIAA1324, THAP2, RAD23B, DDEF1, Probe Set ID 212993, HTLF, KIAA1324, INTS8, PILRB,
EIF4EBP3 /// MASK-BP3, TMEM140, AK5, DNAJC13, DMN, SLC25A13, NCBP1, SERTAD1, STAM,
METTL2B /// METTL2A, JUND, ATP11A, LRP11, ABHD14B, C7orf25, Probe Set ID 228273, STAT1,
LOC643911 /// LOC650242, ACP1, Probe Set ID 221824, SOAT1, ROPN1B, WDR3, UMPS, SLC35B4,
CEBPD, USP9X, RAI2, NFIB, SORBS2, NUDT5, LOC493869, CITED1, E2F8, Probe Set ID 228710,
IARS, ATP2C1, SCGB3A1, CKAP5, PHACTR2, DIAPH3, PTK2, SEC61G, NHSL1, ZBTB20, TNKS,
C1orf63, KDELR3, NUP37, HMMR, RGS2, MAST4, Probe Set ID AFFX-M27830_5, POLE2, CG018,
SCGN, OIP5, CKAP2, PLS3, LOC89944, LOC439994 /// LOC642361 /// LOC646509, AGPS, EIF2S2,
ISG15, FSD1L, MAFF, TXNDC9, SLC16A1, ARPC5, Probe Set ID 241782, Probe Set ID 230863, CDV3,
NIPA2, FLRT3, TPD52, DUSP10, VSIG2, CAMSAP1L1, KIT, PIP, KIAA0103, HS2ST1, HIST1H2BK,
IDH3A, MTSS1, C21orf86, ATIC, PHACTR2, ERCC1, CIRBP, KLF10, MYCBP2, RAD51AP1, LTF ///
LOC643349, MELK, C1orf79, GNPDA1, TOP2A, WDR32, SASS6, SLC44A1, ANKRD13C, SGK3,
FAM45B /// FAM45A, FAM53C, MCAM, TRRAP, RAB31, CHN1, LOC646890, VTCN1, TOB2,
CSNK2A1, LOC283481, BDH2, CMTM7, TICAM2, TM4SF18, Probe Set ID 228049, REEP5,
AMMECR1, THUMPD3, ARSD, ICMT, C1orf107, GTPBP1, RACGAP1, SH3BGRL, NUCKS1, RND1,
SEC23IP, ECOP, EIF4E2, GOPC, WEE1, BBOX1, ETNX1, PODXL, XPR1, FZD2, CCL28, TMEM101,
C11orf32, OLFM4, CSE1L, CSTF1, SCGB1D2, AMMECR1, ARC, PCYT1B, NUSAP1, MAPK14,
C10orf137, GADD45B, IQGAP3, ITPKC, ACAA1, MAPT, EFCAB4A, STK39, FLJ90709, KLHL20,
RNF8, NAV1, SLC24A3, Probe Set ID 230233, C1orf21, DNAPTP6, ECOP, NSF, IRX3, SCYL1BP1,
RRM2, SYT13, GPR180, MRPS14, LOC652689 /// FAM72A /// LOC653594 /// LOC653820, CCNB2,
TUBB, CDK8, MMP7, CLDN4, KIAA0146, CUL5, MTERFD3, UBLCP1, SAMD9, CDCA7, PEBP1,
KLHL12, GABARAPL1 /// GABARAPL3, RUNX1, Probe Set ID 224778, DPF2, TF, Probe Set ID
242354, STAT1, ANGPTL4, JUNB, CNIH4, SEC14L2, Probe Set ID 226348, KDELR3, ETS2,
SOSTDC1, CD24, BCL2, ELP4, GAS2L3, ZWINT, C7orf30, C11orf17 /// NUAK2, HS2ST1, Probe Set ID
238191, ISGF3G, FLJ37453, PGM1, KLHL21, RAB23, NUP133, CEP350, DCUN1D4, AURKA, RMI1,
CDC2, WDR68, SKP2, JTV1, SKIV2L2, Probe Set ID 235247, INTS2, NPAT, CX3CL1, SCNM1,
ATP13A3, CHAC2, CD59, GGT6, ATXN3, Probe Set ID 229174, PSMD5, CEP170, CIRBP, Probe Set ID
225917, DYNLRB2, CLTC, KIF15 /// C7orf9, SRP54, LOC144233, CDCA1, CHPT1, MGC39606 ///
LOC644596, GTF2E1, GADD45B, PSMA5, C1orf107, TMPO, DHRS4, CCDC99, Probe Set ID 243791,
NAT10, CASC5, CDCA3, RBM12B, FOLR1, AIM1, IMPA2, CDC20, CCL28, ERBB4, PAK3 /// UBE2C,
RPE, SNRPN /// SNURF, CAPZA2, MUC15, Probe Set ID 238725, COPB2, ECHDC2, IKBKAP,
GADD45B, MIA, PRSS16, ATP5J2, PAK2, ELL2, TRIM14, UHRF1, Probe Set ID 238824, GPR126,
LMBR1, TBL1XR1, PLSCR1, ASCC3, C11orf2, ARID5A, NGFRAP1L1, CXorf10 /// LOC648176,
CPSF2, EMP1, Probe Set ID 242705_x, ROPN1, CDC42EP5, C16orf33, C6orf107, SHANK2, ERN1,
SLC38A6, LOC255783, SOS2, RALA, JUN, RUNX1, HSPA2, TXNDC9, FADD, DLEU2 /// DLEU2L,
NDEL1, NDRG2, GMPS, AGPS, SATB1, ETS1, JUN, QSER1, CNOT4, H3F3B, SLC24A3, RGC32,
SGOL2, FLJ10038, TTC18, Probe Set ID 228549, STC2, OVOL1, EPHA2, ORC5L, PTN, IRX1, AURKA,
FLJ41603, PI15, MGP, RAB31, PGK1, SNX6, ADCY7, LOH11CR2A, AZI2, WDR37, CLDN8, ANLN,
DEPDC1, Probe Set ID 228854, NEDD9, USP53, SLC25A25, IER5, CYP4V2, SOX10, APLP2, DNAJB1,
FOSB, C15orf23,

From this gene list probes were removed if the probe was not a match to any annotated gene sequences, resulting in a list of 483 genes.

483 GENE LIST

GBE1, ERGIC1, DNMT3A, LDHA, ARSD, DLG7, C4orf32, HK2, MLF1, KIF11, CCNB1, C1orf25,
ZC3HAV1L, OSBPL11, SLC12A8, LAMB1, MND1, ECT2, DBR1, KIAA1217, MATN2, ALKBH1,
METTL7A, ATG3, CXCL2, TMEM106B, FLJ37953, BNIPL, Probe Set ID 226191, CHKB /// CPT1B ///
ARHGAP29, RPS6KC1, HDHD1A, BUB1B, DDEF1, MGAT2, RPL37, ARSD /// DPH5, E2F7, CITED4,
NUCKS1, PLP2, C1orf131, VIL2, LOC285550, IFI27, HNMT, ANKRD25, LOC130576, C10orf7,
WFDC2, GEMIN6, MBD4, ALG2, GINS1, ANPEP, FLNB, C10orf9, GPM6B, UBE2V1 /// Kua-UEV,
KIAA0391, STC2, HN1, C17orf71, FTO, CCDC43, XIST, JUB, SSR1, NUP205, TP53, MGC12966,
C7orf36, ELF5, TBC1D8, RRM2, C1orf121, PDE8A, DSCR1, CCNB1, RRAD, CASP8, ZYG11B,
C14orf125, C4orf7, TMC4, C9orf64, H19, BMSC-MCP, LARS, PEBP1, STAMBPL1, DHX29, BUD31,
PREPL, EPRS, PNKD, MGC7036, MRPS17, SECTM1, SWAP70, C5orf21, PLAA, PARP12, Probe Set ID
219216, NOL8, RAB31, CLIC6, TMF1, AXUD1, GABRP, CX3CL1, GOLGA8A, MCART1, KDELR2,
THEM2, C1orf163, FAM82B, ZDHHC2, Probe Set ID 226347, FAM33A, MRPL15, SAR1A, BRI3BP,
SFPQ, ARSD /// DPH5, CDKN2C, PER2, MAFF, UBE2F, MOBK1B, UBE2T, GAPDH, HSBP1, SFRP1,
KRT15, CEP55, GTF3C3, C12orf4, KIAA1324, RAD23B, DDEF1, Probe Set ID 212993, HTLF,
KIAA1324, INTS8, PILRB, EIF4EBP3 /// MASK-BP3, AK5, DNAJC13, DMN, SLC25A13, NCBP1,
SERTAD1, STAM, METTL2B /// METTL2A, JUND, ATP11A, LRP11, ABHD14B, C7orf25, STAT1,
LOC643911 /// LOC650242, ACP1, Probe Set ID 221824, SOAT1, ROPN1B, WDR3, UMPS, SLC35B4,
CEBPD, USP9X, RAI2, SORBS2, NUDT5, LOC493869, CITED1, E2F8, IARS, ATP2C1, SCGB3A1,
CKAP5, PHACTR2, DIAPH3, PTK2, SEC61G, NHSL1, ZBTB20, TNKS, C1orf63, KDELR3, NUP37,
HMMR, RGS2, MAST4, POLE2, CG018, SCGN, OIP5, CKAP2, PLS3, LOC89944, LOC439994 ///
LOC642361 /// LOC646509, AGPS, EIF2S2, ISG15, FSD1L, MAFF, TXNDC9, SLC16A1, ARPC5, Probe
Set ID 241782, NIPA2, FLRT3, TPD52, DUSP10, VSIG2, CAMSAP1L1, KIT, PIP, KIAA0103, HS2ST1,
HIST1H2BK, IDH3A, MTSS1, C21orf86, ATIC, ERCC1, CIRBP, KLF10, MYCBP2, RAD51AP1, LTF ///
LOC643349, MELK, C1orf79, GNPDA1, TOP2A, WDR32, SLC44A1, ANKRD13C, SGK3, FAM45B ///
FAM45A, FAM53C, TRRAP, RAB31, CHN1, VTCN1, TOB2, CSNK2A1, LOC283481, BDH2, CMTM7,
TICAM2, TM4SF18, REEP5, AMMECR1, THUMPD3, ARSD, ICMT, C1orf107, GTPBP1, RACGAP1,
SH3BGRL, NUCKS1, RND1, SEC23IP, ECOP, EIF4E2, GOPC, WEE1, BBOX1, ETNK1, PODXL,
XPR1, FZD2, CCL28, TMEM101, C11orf32, OLFM4, CSE1L, CSTF1, SCGB1D2, AMMECR1, ARC,
PCYT1B, NUSAP1, MAPK14, C10orf137, GADD45B, IQGAP3, ITPKC, ACAA1, MAPT, EFCAB4A,
STK39, FLJ90709, RNF8, NAV1, SLC24A3, C1orf21, DNAPTP6, ECOP, NSF, IRX3, SCYL1BP1,
RRM2, SYT13, GPR180, MRPS14, LOC652689 /// FAM72A /// LOC653594 /// LOC653820, CCNB2,
TUBB, CDK8, MMP7, CLDN4, MTERFD3, UBLCP1, SAMD9, CDCA7, PEBP1, KLHL12,
GABARAPL1 /// GABARAPL3, Probe Set ID 224778, DPF2, TF, Probe Set ID 242354, STAT1,
ANGPTL4, JUNB, CNIH4, SEC14L2, Probe Set ID 226348, KDELR3, ETS2, SOSTDC1, CD24, BCL2,
ELP4, GAS2L3, ZWINT, C7orf30, C11orf17 /// NUAK2, HS2ST1, ISGF3G, FLJ37453, PGM1, KLHL21,
RAB23, NUP133, CEP350, DCUN1D4, AURKA, RMI1, CDC2, WDR68, JTV1, SKIV2L2, Probe Set ID
235247, INTS2, NPAT, CX3CL1, SCNM1, ATP13A3, CHAC2, CD59, GGT6, ATXN3, Probe Set ID
229174, PSMD5, CEP170, CIRBP, Probe Set ID 225917, DYNLRB2, CLTC, KIF15 /// C7orf9, SRP54,
LOC144233, CDCA1, CHPT1, MGC39606 /// LOC644596, GTF2E1, GADD45B, PSMA5, C1orf107,
TMPO, DHRS4, CCDC99, NAT10, CASC5, CDCA3, RBM12B, FOLR1, AIM1, IMPA2, CDC20, CCL28,
ERBB4, PAK3 /// UBE2C, RPE, SNRPN /// SNURF, CAPZA2, MUC15, Probe Set ID 238725, COPB2,
ECHDC2, IKBKAP, MIA, PRSS16, ATP5J2, PAK2, ELL2, TRIM14, UHRF1, Probe Set ID 238824,
GPR126, LMBR1, TBL1XR1, PLSCR1, ASCC3, C11orf2, ARID5A, NGFRAP1L1, CXorf10 ///
LOC648176, CPSF2, EMP1, Probe Set ID 242705_x, ROPN1, CDC42EP5, C16orf33, C6orf107,
SHANK2, ERN1, SLC38A6, LOC255783, SOS2, RALA, RUNX1, HSPA2, TXNDC9, FADD, DLEU2 ///
DLEU2L, NDEL1, NDRG2, GMPS, AGPS, SATB1, JUN, QSER1, CNOT4, H3F3B, SLC24A3, RGC32,
SGOL2, FLJ10038, TTC18, STC2, OVOL1, EPHA2, ORC5L, PTN, IRX1, AURKA, FLJ41603, PI15,
MGP, RAB31, PGK1, SNX6, ADCY7, LOH11CR2A, AZI2, WDR37, CLDN8, ANLN, DEPDC1,
NEDD9, SLC25A25, IER5, CYP4V2, SOX10, APLP2, DNAJB1, FOSB, C15orf23,

The predictive power of each individual gene from the 483 list was calculated using Cox survival analysis on a set of estrogen receptor positive patients (201 patients) in the GSE3494 dataset. Genes were excluded if they had a high expression associated with good outcome but were up-regulated in tumorigenic samples, or if they had a high expression associated with worse outcome but were down-regulated in tumorigenic samples. If more than one probes matched to one gene, only keep the one with highest expression value (345 genes/probes left).

345 GENE LIST

GBE1, ERGIC1, DNMT3A, LDHA, ARSD, DLG7, C4orf32, HK2, KIF11, OSBPL11, MND1, ECT2,
KIAA1217, ALKBH1, METTL7A, ATG3, CXCL2, FLJ37953, BNIPL, CHKB /// CPT1B /// ARHGAP29,
RPS6KC1, HDHD1A, BUB1B, DDEF1, E2F7, CITED4, NUCKS1, PLP2, C1orf131, LOC285550, IFI27,
HNMT, LOC130576, C10orf7, WFDC2, GEMIN6, MBD4, GINS1, ANPEP, FLNB, C10orf9, GPM6B,
UBE2V1 /// Kua-UEV, KIAA0391, STC2, HN1, C17orf71, FTO, CCDC43, XIST, JUB, SSR1, TP53,
MGC12966, C7orf36, ELF5, RRM2, DSCR1, CCNB1, RRAD, CASP8, C4orf7, TMC4, BMSC-MCP,
LARS, DHX29, BUD31, EPRS, PNKD, MGC7036, MRPS17, SWAP70, C5orf21, PLAA, PARP12,
NOL8, CLIC6, TMF1, AXUD1, GABRP, GOLGA8A, KDELR2, THEM2, C1orf163, FAM82B,
ZDHHC2, FAM33A, MRPL15, SAR1A, BRI3BP, SFPQ, UBE2F, MOBK1B, UBE2T, GAPDH, SFRP1,
KRT15, CEP55, GTF3C3, C12orf4, RAD23B, HTLF, KIAA1324, INTS8, PILRB, EIF4EBP3 /// MASK-
BP3, AK5, DMN, SLC25A13, NCBP1, STAM, METTL2B /// METTL2A, LRP11, LOC643911 ///
LOC650242, ACP1, Probe Set ID 221824, ROPN1B, WDR3, UMPS, CEBPD, USP9X, RAI2, SORBS2,
NUDT5, E2F8, IARS, SCGB3A1, CKAP5, DIAPH3, PTK2, SEC61G, ZBTB20, C1orf63, KDELR3,
NUP37, HMMR, RGS2, POLE2, CG018, OIP5, CKAP2, LOC89944, LOC439994 /// LOC642361 ///
LOC646509, AGPS, EIF2S2, ISG15, FSD1L, MAFF, TXNDC9, ARPC5, NIPA2, TPD52, VSIG2, KIT,
PIP, KIAA0103, HS2ST1, HIST1H2BK, IDH3A, MTSS1, ATIC, ERCC1, KLF10, RAD51AP1, LTF ///
LOC643349, MELK, GNPDA1, TOP2A, SLC44A1, SGK3, FAM45B /// FAM45A, TRRAP, VTCN1,
CSNK2A1, LOC283481, BDH2, CMTM7, TM4SF18, REEP5, THUMPD3, ICMT, C1orf107, RACGAP1,
SH3BGRL, NUCKS1, SEC23IP, ECOP, EIF4E2, BBOX1, ETNK1, PODXL, FZD2, CCL28, TMEM101,
C11orf32, CSE1L, CSTF1, AMMECR1, ARC, NUSAP1, IQGAP3, MAPT, EFCAB4A, STK39, RNF8,
C1orf21, NSF, IRX3, SYT13, GPR180, MRPS14, LOC652689 /// FAM72A /// LOC653594 ///
LOC653820, CCNB2, TUBB, CDK8, MMP7, MTERFD3, SAMD9, CDCA7, KLHL12, GABARAPL1 ///
GABARAPL3, Probe Set ID 224778, TF, Probe Set ID 242354, STAT1, ANGPTL4, JUNB, CNIH4,
SEC14L2, ETS2, SOSTDC1, BCL2, ELP4, GAS2L3, ZWINT, C7orf30, C11orf17 /// NUAK2, ISGF3G,
FLJ37453, PGM1, KLHL21, NUP133, CEP350, AURKA, RMI1, CDC2, WDR68, JTV1, SKIV2L2, Probe
Set ID 235247, INTS2, CX3CL1, SCNM1, ATP13A3, CHAC2, CD59, GGT6, ATXN3, Probe Set ID
229174, PSMD5, CEP170, CIRBP, Probe Set ID 225917, DYNLRB2, CLTC, KIF15 /// C7orf9, SRP54,
CDCA1, CHPT1, MGC39606 /// LOC644596, GTF2E1, GADD45B, PSMA5, TMPO, CCDC99, NAT10,
CASC5, CDCA3, CDC20, ERBB4, PAK3 /// UBE2C, RPE, SNRPN /// SNURF, CAPZA2, COPB2,
ECHDC2, IKBKAP, MIA, PRSS16, ATP5J2, PAK2, UHRF1, Probe Set ID 238824, GPR126, TBL1XR1,
ASCC3, C11orf2, ARID5A, CPSF2, EMP1, ROPN1, CDC42EP5, C16orf33, C6orf107, SHANK2, ERN1,
SLC38A6, LOC255783, SOS2, RALA, RUNX1, HSPA2, FADD, NDEL1, NDRG2, GMPS, AGPS,
SATB1, JUN, QSER1, CNOT4, H3F3B, RGC32, SGOL2, FLJ10038, TTC18, ORC5L, PTN, IRX1,
FLJ41603, PI15, MGP, PGK1, SNX6, LOH11CR2A, AZI2, CLDN8, ANLN, DEPDC1, NEDD9,
CYP4V2, SOX10, APLP2, FOSB, C15orf23,

In some embodiments, the remaining 345 genes were ranked by P value of Cox survival analysis from lowest to highest, generating gene signature from the 10, 11, 12, . . . 345 genes. The predictive power of these gene signatures was tested on the GSE 3494 ER+ patient population, and the gene signature with the lowest P value was chosen: a 52 gene cancer stem cell gene signature 4 (Table 4A). Because there was not a one-to-one correspondence between the generated gene signatures and the arrays used to analyze expression of the different patient populations, a separate gene profile was generated as shown in Tables 4B,C below for each dataset without a complete gene signature.

TABLE 4A
Cancer Stem Cell Gene Signature 4

Elevated Expression
EIF4E2, C11orf17 /// NUAK2, FADD, E2F8, RALA, PGK1, NUDT5, GEMIN6, PLP2, ACP1, MRPS17,
KIF15 /// C7orf9, UBE2V1 /// Kua-UEV, IQGAP3, SGOL2, PAK3 /// UBE2C, OIP5, UBE2T, ZWINT,
DIAPH3, CCNB2, CSE1L, CKAP2, UHRF1, BUB1B, CDC20, KIF11, GINS1, CEP55, MELK,
RACGAP1, CCNB1, NUSAP1, CDC2, RRM2, DLG7
Reduced Expression
XIST, RGC32, FOSB, CLIC6, STC2, MAPT, KLHL21, SATB1, BNIPL, RUNX1, PODXL, FLJ41603,
ZBTB20, SEC14L2, CG018, SWAP70

TABLE 4B
Cancer Gene Profile 4A Generated from the NKI Array

DLG7, KIF11, BNIPL, BUB1B, PLP2, GEMIN6, STC2, RRM2, CCNB1, MRPS17, CLIC6, ACP1,
DIAPH3, ZBTB20, CG018, OIP5, CKAP2, MELK, RACGAP1, EIF4E2, PODXL, CSE1L, NUSAP1,
MAPT, CCNB2, SEC14L2, ZWINT, KLHL21, CDC2, CDC20, UHRF1, RALA, FADD, SATB1, RGC32,
SGOL2, FLJ41603, PGK1, FOSB

TABLE 4C
Cancer Gene Profile 4B Generated from
the GSE2034 and GSE2990 Array

DLG7, KIF11, BUB1B, PLP2, GEMIN6, GINS1, UBE2V1///Kua-UEV,
STC2, RRM2, CCNB1, MRPS17, SWAP70, CEP55, ACP1, E2F8,
ZBTB20, CG018, OIP5, CKAP2, MELK, RACGAP1, EIF4E2, PODXL,
CSE1L, NUSAP1, MAPT, CCNB2, SEC14L2, ZWINT, C11orf17///
NUAK2, KLHL21, CDC2, KIF15///C7orf9, CDC20, PAK3///UBE2C,
RALA, RUNX1, FADD, SATB1, RGC32, PGK1, FOSB

In some embodiments, the remaining 345 genes were separated into two lists: 1) those that were up-regulated in tumorigenic versus non-tumorigenic samples and 2) those that were down-regulated in tumorigenic versus non-tumorigenic samples. Each gene in these separate lists was ranked by P value from lowest to highest, generating gene signature from the lowest 4, 11, 12, . . . 50 genes combined from each list. The predictive power of these gene signatures was tested on the GSE 3494 ER+ patient population, and the gene signature with the lowest P value was chosen: a 34 gene cancer stem cell gene signature 5 with 17 up-regulated and 17 down-regulated genes (Table 5A). Because there was not a one-to-one correspondence between the generated gene signatures and the arrays used to analyze expression of the different patient populations, a separate gene profile was generated as shown in Tables 5B,C below for each dataset without a complete gene signature.

TABLE 5A
Cancer Stem Cell Gene Signature 5

Elevated Expression
IQGAP3, MELK, UBE2V1///Kua-UEV, CDC2, KIF11, RALA, RRM2,
FADD, CCNB1, CCNB2, BUB1B, RACGAP1, EIF4E2, PAK3///UBE2C,
ZWINT, GINS1, NUSAP1
Reduced Expression
MAPT, STC2, XIST, ZBTB20, RUNX1, CG018, SATB1, SFRP1,
BNIPL, RGC32, FLJ41603, SEC14L2, PODXL, SWAP70, CLIC6,
KLHL21, FOSB

TABLE 5B
Cancer Gene Profile 5A Generated from the NKI Array

MELK, CDC2, MAPT, STC2, KIF11, ZBTB20, RALA, RUNX1, RRM2,
FADD, CG018, CCNB1, SATB1, SFRP1, BNIPL, CCNB2, RGC32,
BUB1B, RACGAP1, FLJ41603, SEC14L2, EIF4E2, PODXL,
ZWINT, CLIC6, KLHL21, FOSB, NUSAP1

TABLE 5C
Cancer Gene Profile 5B Generated from
the GSE2034 and GSE2990 Arrays

MELK, UBE2V1///Kua-UEV, CDC2, MAPT, STC2, KIF11, ZBTB20,
RALA, RUNX1, RRM2, FADD, CG018, CCNB1, SATB1, SFRP1,
CCNB2, RGC32, BUB1B, RACGAP1, SEC14L2, EIF4E2, PAK3 ///
UBE2C, PODXL, SWAP70, ZWINT, KLHL21, GINS1, FOSB, NUSAP1

In some embodiments, from the 345 gene list, genes with expression levels below the median value in tumorigenic samples or in normal breast samples were removed, leaving 242 genes.

242 GENE LIST

GBE1, ERGIC1, DNMT3A, LDHA, ARSD, C4orf32, HK2, KIAA1217, METTL7A, ATG3, CXCL2,
FLJ37953, RPS6KC1, HDHD1A, DDEF1, CITED4, NUCKS1, PLP2, C1orf131, LOC285550,
LOC130576, C10orf7, WFDC2, GEMIN6, MBD4, ANPEP, FLNB, C10orf9, UBE2V1///Kua-UEV,
KIAA0391, STC2, HN1, C17orf71, FTO, CCDC43, JUB, SSR1, TP53, MGC12966, C7orf36, RRM2,
DSCR1, CASP8, TMC4,, BMSC-MCP, LARS, DHX29, BUD31, EPRS, PNKD, MGC7036, MRPS17,
SWAP70, PARP12, NOL8, CLIC6, TMF1, AXUD1, GABRP, KDELR2, THEM2, C1orf163, FAM82B,
FAM33A, MRPL15, SAR1A, BRI3BP, SFPQ, UBE2F, UBE2T, GAPDH, SFRP1, GTF3C3, C12orf4,
RAD23B, HTLF, KIAA1324, INTS8, PILRB, EIF4EBP3///MASK-BP3, DMN, SLC25A13, STAM,
LRP11, LOC643911///LOC650242, ACP1, Probe Set ID 221824, WDR3, UMPS, CEBPD, USP9X,
NUDT5, IARS, SCGB3A1, CKAP5, DIAPH3, PTK2, SEC61G, ZBTB20, C1orf63, NUP37, RGS2,
POLE2, LOC89944, LOC439994///LOC642361///LOC646509, EIF2S2, ISG15, MAFF, TXNDC9,
ARPC5, NIPA2, TPD52, PIP, KIAA0103, HS2ST1, HIST1H2BK, MTSS1, ATIC, ERCC1, KLF10, LTF
/// LOC643349, GNPDA1, SLC44A1, SGK3, FAM45B///FAM45A, VTCN1, CSNK2A1, BDH2,
CMTM7, REEP5, THUMPD3, ICMT, C1orf107, SH3BGRL, NUCKS1, SEC23IP, ECOP, EIF4E2,
PODXL, CCL28, C11orf32, CSE1L, NUSAP1, IQGAP3, MAPT, STK39, RNF8, C1orf21, NSF, IRX3,
SYT13, GPR180, MRPS14, CCNB2, TUBB, MMP7, MTERFD3, KLHL12, GABARAPL1 ///
GABARAPL3, Probe Set ID 224778, Probe Set ID 242354, STAT1, JUNB, CNIH4, ETS2, BCL2, ELP4,
ZWINT, C7orf30, C11orf17///NUAK2, ISGF3G, FLJ37453, PGM1, NUP133, CEP350, AURKA, RMI1,
WDR68, JTV1, SKIV2L2, CX3CL1, SCNM1, ATP13A3, CD59, GGT6, ATXN3, PSMD5, CIRBP, Probe
Set ID 225917, CLTC, SRP54, CHPT1, GTF2E1, GADD45B, PSMA5, TMPO, NAT10, CDCA3, ERBB4,
PAK3///UBE2C, RPE, SNRPN///SNURF, CAPZA2, COPB2, ECHDC2, IKBKAP, PRSS16, ATP5J2,
Probe Set ID 238824, TBL1XR1, C11orf2, ARID5A, CPSF2, EMP1, C16orf33, C6orf107, ERN1,
SLC38A6, LOC255783, SOS2, HSPA2, FADD, NDEL1, NDRG2, GMPS, SATB1, JUN, QSER1, CNOT4,
H3F3B, RGC32, FLJ10038, ORC5L, FLJ41603, MGP, PGK1, SNX6, NEDD9, SOX10, APLP2, FOSB,
C15orf23

These 242 remaining genes were separated into two lists: 1) those that were up-regulated in tumorigenic versus non-tumorigenic samples and 2) those that were down-regulated in tumorigenic versus non-tumorigenic samples. Each gene in these separate lists was ranked by P value from lowest to highest, generating gene signature from the lowest 4, 11, 12, . . . 50 genes combined from each list. The predictive power of these gene signatures was tested on the GSE 3494 ER+ patient population, and the gene signature with the lowest P value was chosen: a 26 gene cancer stem cell gene signature 6 with 13 up-regulated and 13 down-regulated genes (Table 6A). Because there was not a one-to-one correspondence between the generated gene signatures and the arrays used to analyze expression of the different patient populations, a separate gene profile was generated as shown in Tables 6B,C below for each dataset without a complete gene signature.

TABLE 6A
Cancer Stem Cell Gene Signature 6

Elevated Expression
IQGAP3, UBE2V1///Kua-UEV, DIAPH3, RRM2, FADD, UBE2T,
CCNB2, PLP2, EIF4E2, PAK3///UBE2C, ZWINT, ACP1, NUSAP1
Reduced Expression
MAPT, STC2, ZBTB20, SATB1, SFRP1, RGC32, FLJ41603, PODXL,
SWAP70, ECHDC2, CLIC6, PRSS16, FOSB

TABLE 6B
Cancer Gene Profile 6A Generated from the NKI Array

MAPT, STC2, DIAPH3, ZBTB20, RRM2, FADD, SATB1, SFRP1,
CCNB2, RGC32, FLJ41603, PLP2, EIF4E2, PODXL, ZWINT,
ECHDC2, CLIC6, PRSS16, FOSB, ACP1, NUSAP1

TABLE 6C
Cancer Gene Profile 6B Generated from
the GSE2034 and GSE2990 Arrays

UBE2V1///Kua-UEV, MAPT, STC2, ZBTB20, RRM2, FADD, SATB1,
SFRP1, CCNB2, RGC32, PLP2, EIF4E2, PAK3///UBE2C, PODXL,
SWAP70, ZWINT, ECHDC2, PRSS16, FOSB, ACP1, NUSAP1

In some embodiments, the predictive power of each individual gene from the 483 list above was calculated using Cox survival analysis on a set of estrogen receptor positive patients from the combined GSE3494/GSE1456 datasets. Genes were excluded if they had a high expression associated with good outcome but were up-regulated in tumorigenic samples, or if they had a high expression associated with worse outcome but were down-regulated in tumorigenic samples. If more than one probes matched to one gene, only keep the one with highest expression value (347 genes/probes left).

347 GENE LIST

GBE1, ERGIC1, DNMT3A, LDHA, ARSD, DLG7, C4orf32, HK2, KIF11, C1orf25, OSBPL11, MND1,
ECT2, DBR1, KIAA1217, ALKBH1, METTL7A, ATG3, CXCL2, TMEM106B, FLJ37953, BNIPL, Probe
Set ID 226191, CHKB///CPT1B///ARHGAP29, RPS6KC1, HDHD1A, BUB1B, DDEF1, RPL37, E2F7,
CITED4, PLP2, C1orf131, LOC285550, IFI27, HNMT, LOC130576, C10orf7, WFDC2, GEMIN6, GINS1,
ANPEP, FLNB, GPM6B, UBE2V1///Kua-UEV, KIAA0391, STC2, HN1, C17orf71, FTO, CCDC43,
XIST, JUB, SSR1, NUP205, MGC12966, C7orf36, ELF5, TBC1D8, RRM2, C1orf121, CCNB1, RRAD,
CASP8, C4orf7, BMSC-MCP, LARS, DHX29, BUD31, PREPL, EPRS, PNKD, MGC7036, MRPS17,
SECTM1, SWAP70, C5orf21, PLAA, PARP12, CLIC6, TMF1, AXUD1, GABRP, GOLGA8A, KDELR2,
THEM2, C1orf163, FAM82B, ZDHHC2, FAM33A, MRPL15, SAR1A, BRI3BP, SFPQ, UBE2F,
MOBK1B, UBE2T, GAPDH, SFRP1, KRT15, CEP55, C12orf4, RAD23B, HTLF, KIAA1324, INTS8,
PILRB, EIF4EBP3///MASK-BP3, AK5, DMN, SLC25A13, NCBP1, STAM, METTL2B///METTL2A,
ATP11A, LRP11, ACP1, Probe Set ID 221824, ROPN1B, WDR3, UMPS, CEBPD, USP9X, RAI2,
SORBS2, NUDT5, CITED1, E2F8, IARS, ATP2C1, SCGB3A1, CKAP5, DIAPH3, SEC61G, ZBTB20,
C1orf63, KDELR3, NUP37, HMMR, RGS2, POLE2, CG018, OIP5, CKAP2, LOC89944, LOC439994 ///
LOC642361///LOC646509, AGPS, EIF2S2, ISG15, FSD1L, MAFF, TXNDC9, ARPC5, NIPA2, VSIG2,
KIT, PIP, KIAA0103, HS2ST1, HIST1H2BK, IDH3A, MTSS1, C21orf86, ATIC, ERCC1, KLF10,
RAD51AP1, LTF///LOC643349, MELK, TOP2A, SLC44A1, SGK3, FAM45B///FAM45A, TRRAP,
CHN1, VTCN1, TOB2, CSNK2A1, LOC283481, BDH2, CMTM7, TM4SF18, REEP5, THUMPD3,
ICMT, C1orf107, RACGAP1, SH3BGRL, NUCKS1, SEC23IP, ECOP, EIF4E2, BBOX1, PODXL,
CCL28, TMEM101, C11orf32, OLFM4, CSE1L, CSTF1, AMMECR1, PCYT1B, NUSAP1, IQGAP3,
ACAA1, MAPT, EFCAB4A, STK39, RNF8, C1orf21, NSF, IRX3, SYT13, GPR180, MRPS14,
LOC652689///FAM72A///LOC653594///LOC653820, CCNB2, TUBB, CDK8, MMP7, CDCA7,
KLHL12, GABARAPL1///GABARAPL3, Probe Set ID 224778, TF, Probe Set ID 242354, STAT1,
ANGPTL4, JUNB, CNIH4, SEC14L2, ETS2, SOSTDC1, BCL2, ELP4, GAS2L3, ZWINT, C7orf30,
C11orf17///NUAK2, ISGF3G, FLJ37453, PGM1, KLHL21, NUP133, DCUN1D4, AURKA, RMI1,
CDC2, WDR68, JTV1, SKIV2L2, Probe Set ID 235247, INTS2, CX3CL1, SCNM1, ATP13A3, CHAC2,
CD59, GGT6, ATXN3, Probe Set ID 229174, PSMD5, CIRBP, Probe Set ID 225917, DYNLRB2, CLTC,
KIF15///C7orf9, SRP54, LOC144233, CDCA1, CHPT1, MGC39606 /// LOC644596, GTF2E1,
GADD45B, PSMA5, C1orf107, TMPO, CCDC99, NAT10, CASC5, CDCA3, IMPA2, CDC20, CCL28,
ERBB4, PAK3///UBE2C, RPE, SNRPN///SNURF, COPB2, ECHDC2, IKBKAP, MIA, PRSS16,
ATP5J2, PAK2, UHRF1, Probe Set ID 238824, GPR126, ASCC3, C11orf2, ARID5A, CPSF2, EMP1,
ROPN1, CDC42EP5, C16orf33, C6orf107, SHANK2, SLC38A6, LOC255783, SOS2, RALA, RUNX1,
HSPA2, FADD, NDEL1, NDRG2, GMPS, SATB1, JUN, QSER1, H3F3B, RGC32, SGOL2, FLJ10038,
TTC18, EPHA2, ORC5L, PTN, IRX1, FLJ41603, PI15, MGP, PGK1, SNX6, LOH11CR2A, AZI2,
CLDN8, ANLN, DEPDC1, NEDD9, SLC25A25, CYP4V2, SOX10, APLP2, FOSB, C15orf23

In some embodiments, the remaining 347 genes were ranked by P value of Cox survival analysis from lowest to highest, generating gene signature from the 10, 11, 12, . . . 347 genes. The predictive power of these gene signatures was tested on the GSE 3494/GSE1456 ER+ patient population, and the gene signature with the lowest P value was chosen: a 74 gene cancer stem cell gene signature 7 (Table 7A). Because there was not a one-to-one correspondence between the generated gene signatures and the arrays used to analyze expression of the different patient populations, a separate gene profile was generated as shown in Tables 7B,C below for each dataset without a complete gene signature.

TABLE 7A
Cancer Stem Cell Gene Signature 7

Elevated Expression
RAD51AP1, IQGAP3, DNMT3A, MELK, LDHA, UBE2V1///Kua-UEV,
TOP2A, CDC2, NUDT5, DLG7, HN1, E2F8, CKAP5, DIAPH3,
SEC61G, KIF11, RALA, HMMR, ECT2, GAPDH, RRM2, FADD,
UBE2T, OIP5, CKAP2, CCNB1, GMPS, LOC652689///FAM72A///
LOC653594///LOC653820, CCNB2, CEP55, KIF15///C7orf9,
CDCA1, SGOL2, BUB1B, CASC5, CDCA7, RACGAP1, CDCA3,
E2F7, PLP2, EPRS, EIF4E2, PGK1, MRPS17, CDC20, NCBP1,
PAK3 /// UBE2C, ANLN, DEPDC1, ZWINT, IDH3A, CSE1L,
GEMIN6, GINS1, ACP1, UHRF1, NUSAP1, THEM2, C15orf23,
AURKA
Reduced Expression
ARID5A, RAI2, MAPT, ZBTB20, RUNX1, CG018, SFRP1, RGC32,
FLJ41603, PODXL, SWAP70, CLIC6, KLHL21, FOSB

TABLE 7B
Cancer Gene Profile 7A Generated from the NKI Array

RAD51AP1, RAI2, DNMT3A, MELK, LDHA, TOP2A, CDC2, DLG7,
MAPT, HN1, DIAPH3, SEC61G, KIF11, ZBTB20, RALA, RUNX1,
HMMR, ECT2, RRM2, FADD, CG018, OIP5, CKAP2, CCNB1,
GMPS, SFRP1, CCNB2, CDCA1, RGC32, SGOL2, BUB1B, CDCA7,
RACGAP1, FLJ41603, CDCA3, E2F7, PLP2, EPRS, EIF4E2,
PGK1, MRPS17, CDC20, NCBP1, PODXL, ANLN, ZWINT, IDH3A,
CLIC6, CSE1L, GEMIN6, KLHL21, FOSB, ACP1, UHRF1,
NUSAP1, THEM2, C15orf23

TABLE 7C
Cancer Gene Profile 7B Generated from
the GSE2034 and GSE2990 Arrays

RAD51AP1, ARID5A, RAI2, MELK, LDHA, UBE2V1///Kua-UEV,
TOP2A, CDC2, DLG7, MAPT, HN1, E2F8, CKAP5, SEC61G,
KIF11, ZBTB20, RALA, RUNX1, HMMR, ECT2, GAPDH, RRM2,
FADD, CG018, OIP5, CKAP2, CCNB1, GMPS, SFRP1, CCNB2,
CEP55, KIF15///C7orf9, RGC32,, BUB1B, RACGAP1, PLP2,
EPRS, EIF4E2, PGK1, MRPS17, CDC20, NCBP1, PAK3///
UBE2C, PODXL, SWAP70, ZWINT, IDH3A, CSE1L, GEMIN6,
KLHL21, GINS1, FOSB, ACP1, NUSAP1, THEM2, AURKA

In some embodiments, the remaining 347 genes were separated into two lists: 1) those that were up-regulated in tumorigenic versus non-tumorigenic samples and 2) those that were down-regulated in tumorigenic versus non-tumorigenic samples. Each gene in these separate lists was ranked by P value of Cox survival analysis from lowest to highest, generating gene signature from the lowest 4, 11, 12, . . . 50 genes combined from each list. The predictive power of these gene signatures was tested on the combined GSE 3494/1456 ER+ patient population, and the gene signature with the lowest P value was chosen: a 16 gene cancer stem cell gene signature 8 with 8 up-regulated and 8 down-regulated genes (Table 8A). Because there was not a one-to-one correspondence between the generated gene signatures and the arrays used to analyze expression of the different patient populations, a separate gene profile was generated as shown in Tables 8B,C below for each dataset without a complete gene signature.

TABLE 8A
Cancer Stem Cell Gene Signature 8

Elevated Expression
IQGAP3, DLG7, RRM2, CCNB1, CCNB2, RACGAP1, PAK3///
UBE2C, NUSAP1
Reduced Expression
ARID5A, MAPT, ZBTB20, RUNX1, CG018, PODXL, SWAP70,
KLHL21

TABLE 8B
Cancer Gene Profile 8A Generated from the NKI Array

DLG7, MAPT, ZBTB20, RUNX1, RRM2, CG018, CCNB1, CCNB2,
RACGAP1, PODXL, KLHL21, NUSAP1

TABLE 8C
Cancer Gene Profile 8B Generated from
the GSE2034 and GSE2990 Arrays

ARID5A, DLG7, MAPT, ZBTB20, RUNX1, RRM2, CG018, CCNB1,
CCNB2, RACGAP1, PAK3 ///UBE2C, PODXL, SWAP70, KLHL21,
NUSAP1

The invention further embodies the use of these tumor stem cell gene signatures to predict clinical outcome including, but not limited to, metastasis and death. Any independent patient population that includes gene expression analysis (e.g. microarray analysis, immunohistochemical analysis, etc) or tumor samples suitable for gene expression analysis (e.g. frozen tissue biopsies, paraffin embedded tumor tissue samples, etc) along with determined clinical parameters or ongoing monitoring of clinical parameters including, for example, lymph node status, metastasis, death, etc. can be used to assess the ability of a tumor stem cell gene signature to predict clinical outcomes.

The invention therefore establishes multiple cancer stem cell gene signatures, as predictors of poor clinical outcome. In some embodiments of the present invention these cancer stem cell gene signatures are used clinically to classify tumors as low or high risk and to assign a tumor to a low or high-risk category. The cancer stem cell gene signatures can further be used to provide a diagnosis, prognosis, and/or select a therapy based on the classification of a tumor as low or high risk as well as to monitor a diagnosis, prognosis, and/or therapy over time. If it is known that a patient has a tumor that expresses the genes comprising a cancer stem cell gene signature and thus has a poor prognosis, a more aggressive approach to therapy can be warranted than in tumors not falling within these subcategories. For example, in patients where there is no evidence of disease in lymph nodes (node-negative patients), a decision must be made regarding whether to administer chemotherapy (adjuvant therapy) following surgical removal of the tumor. While some patients are likely to benefit from such treatment, it has significant side effects and is preferably avoided by patients with low risk tumors. Presently it is difficult or impossible to predict which patients would benefit. Knowing that a patient falls into a poor prognosis category can help in this decision. Furthermore, detecting expression of a cancer gene profile that is highly correlated with a cancer stem cell gene signature of the present invention can provide information related to tumor progression. It is well known that as tumors progress, their phenotypic characteristics can change. The invention thus contemplates the possibility that breast tumors can evolve from expressing a cancer gene profile that is highly correlated with a cancer stem cell gene signature to not (or vice versa) either in response to therapy or in response to lack of therapy. Thus detection of a cancer gene profile that either correlates with or fails to correlate with a cancer stem cell gene signature can be used to detect such progression and alter therapy accordingly.

It is well known in the art that some tumors respond to certain therapies while others do not. At present there is very little information that can be used to determine, prior to treatment, the likelihood that a specific tumor will respond to a given therapeutic agent. Many compounds have been tested for anti-tumor activity and appear to be effective in only a small percentage of tumors. Due to the current inability to predict which tumors will respond to a given agent, these compounds have not been developed as therapeutics. This problem reflects the fact that current methods of classifying tumors are limited. However, the present invention offers the possibility of identifying tumor subgroups and characterizing tumors by a significant likelihood of response to a given agent. Tumor sample archives containing tissue samples obtained from patients that have undergone therapy with various agents are available along with information regarding the results of such therapy. In general such archives consist of tumor samples embedded in paraffin blocks. These tumor samples can be analyzed for their expression of polypeptides that are then compared to the polypeptides encoded by the genes comprising a cancer stem cell signature of the present invention. For example, immunohistochemistry can be performed using antibodies that bind to the polypeptides. Alternatively these tumor samples can be analyzed by their expression of polynucleotides that are then compared to the polynucleotides encoded by the genes comprising a cancer stem cell signature of the present invention. For example, RNA can be extracted from the tumor sample and RT-PCR used to quantitatively amplify mRNAs that would then be compared to the mRNAs comprising a cancer stem cell signature. Tumors belonging to one or more of thirteen cancer stem cell subclasses can be identified on the basis of this information. It is then possible to correlate the expression of the cancer gene profile with a cancer stem cell gene signature predicted response of the tumor to therapy, thereby identifying particular compounds that show a superior efficacy against tumors of a certain subclass as compared with their efficacy against tumors overall or against tumors not falling within the subclass. Once such compounds are identified it will be possible to select patients whose tumors fall into a particular subclass for additional clinical trials using these compounds. Such clinical trials, performed on a selected group of patients, are more likely to demonstrate efficacy. The reagents provided herein, therefore, are valuable both for retrospective and prospective trials.

In the case of prospective trials, detection of expression of one or more of the genes or encoded proteins in a cancer gene profile that correlates with a cancer stem cell signature can be used to stratify patients prior to their entry into the trial or while they are enrolled in the trial. In clinical research, stratification is the process or result of describing or separating a patient population into more homogeneous subpopulations according to specified criteria. Stratifying patients initially rather than after the trial is frequently some (including by regulatory agencies such as the U.S. Food and Drug Administration involved in the approval process for a medication), and stratification is frequently useful in performing statistical analysis of the results of a trial. In some cases stratification can be required by the study design. Various stratification criteria can be employed in conjunction with detection of expression of one or more cancer gene profiles that correlate with a cancer stem cell gene signature. Commonly used criteria include age, family history, lymph node status, tumor size, tumor grade, etc. Other criteria that can be used include, but are not limited to, tumor aggressiveness, prior therapy received by the patient, estrogen receptor (ER) and/or progesterone receptor (PR) positivity, Her2/neu status, p53 status, etc. Ultimately, once compounds that exhibit superior efficacy against cancer gene profile tumors that are highly correlated with cancer stem cell gene signature are identified, reagents for detecting expression of the gene profile can be used to guide the selection of appropriate therapy for additional patients. Thus, by providing reagents and methods for classifying tumors based on their expression of a cancer gene profile that is compared to a cancer stem cell gene signature, the present invention provides a means to identify a patient population that can benefit from potentially promising therapies that have been abandoned due to inability to benefit broader or more heterogeneous patient populations and further offers a means to individualize cancer therapy.

Information regarding the expression of cancer stem cell signature genes is thus useful even in the absence of specific information regarding their biological function or role in tumor development, progression, and maintenance. Although the reagents disclosed herein find particular application with respect to breast cancer, the invention also contemplates their use to provide diagnostic and/or prognostic information for other cancer types including but not limited to: biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and modullar carcinoma; and renal cancer including adenocarcinoma and Wilms tumor.

In other embodiments of the present invention, the cancer stem cell signatures are used experimentally to test and assess lead compounds including, for example, small molecules, siRNAs, and antibodies for the treatment of cancer. For example tumor cells from a patient can be screened for expression of a cancer stem cell gene signature and then transplanted into the xenograft model described herein and the effect of test compounds, such as for example antibodies against one or more cancer stem cell markers described herein, tested for effects on tumor growth and survival. Furthermore a cancer gene profile can be determined following treatment and the cancer gene profile compared to a cancer stem cell gene signature to assess the effectiveness of the therapy and in turn guide a future treatment regimen. In addition the efficacy of test compounds can be assessed against different tumor subclasses. For example test compounds can be used in xenografts of tumors that express a cancer gene profile that is highly correlated with a tumor stem cell gene signature versus tumors having a gene profile that does not correlate with the tumor stem cell gene signature or that express other gene signature such as, for example, a serum or wound response gene signature (Chang et al., 2005, PNAS 102:3738). Any differences in response of the different tumor subclasses to the test compound are determined and used to optimize treatment for particular classes of tumors.

The cancer stem cell gene signatures were identified from genes that are expressed at decreased or elevated levels in tumor stem cells compared to normal breast epithelium. Thus in certain embodiments of the invention expression levels of mRNA, or amplified or cloned version thereof, are determined from a tumor sample by hybridization to polynucleotides that represent each particular gene comprising a cancer stem cell gene signature. Some polynucleotides of this type contain at least about 20 to at least about 32 consecutive basepairs of a gene sequence that is not found in other gene sequences. Even more some are polynucleotides of at least or about 50 to at least or about 400 basepairs of a gene sequence that is not found in other gene sequences. Such polynucleotides are also referred to as polynucleotide probes in that they are capable of hybridizing to sequences of the genes, or unique portions thereof, described herein. The sequences can be those of mRNA encoded by the genes, the corresponding cDNA to such mRNAs, and/or amplified versions of such sequences. In one some embodiment of the invention a cancer gene profile is detected by polynucleotide probes that comprise the polynucleotides comprising the stem cell gene signature immobilized on an array (such as a cDNA microarray).

In another some embodiment of the invention, all or part of the disclosed polynucleotides of a cancer stem cell gene signature are amplified and detected by methods such as the polymerase chain reaction (PCR) and variations thereof, such as, but not limited to, quantitative PCR (Q-PCR), reverse transcription PCR (RT-PCR), and real-time PCR (including means of measuring the initial amounts of mRNA copies for each sequence in a sample). Real-time RT-PCR or real-time Q-PCR can be used. Such methods utilize one or two primers that are complementary and hybridize to portions of a disclosed sequence, where the primers are used to prime nucleic acid synthesis. The newly synthesized nucleic acids are optionally labeled and can be detected directly or by hybridization to a polynucleotide of the invention. Additional methods to detect expressed nucleic acids include RNAse protection assays, including liquid phase hybridizations, and in situ hybridization of cells or tissue samples.

In yet other embodiments of the invention, gene expression can be determined by analysis of protein expression. Protein expression can be detected by use of one or more antibodies specific for one or more epitopes of individual gene products (proteins), or proteolytic fragments thereof, of a cancer stem cell signature in a tumor sample. Detection methodologies suitable for use in the practice of the invention include, but are not limited to, immunohistochemistry of cells in a tumor sample, enzyme linked immunosorbent assays (ELISAs) including antibody sandwich assays of cells in a tumor sample, mass spectroscopy, immuno-PCR, FACS, and protein microarrays.

It is envisioned that any patient sample can be used to detect a cancer stem cell signature. Importantly, though the cancer stem cell signatures were discovered from a comparison of cancer stem cells against a non-tumorigenic tissue, such as for example, normal breast tissue, its prognostic ability was identified from microarray analysis of unfractionated, and thus heterogeneous, breast tumor samples normalized either against a reference set of tumor samples (van't Veer et al., 2002, Nature 415:530; van de Vijver et al., 2002, N. Eng. J. Med. 347:1999) or to a target intensity (Wang et al., 2005, Lancet 365:671). Thus unfractioned tumor samples, including but not limited to a solid tissue biopsy, fine needle aspiration, or pleural effusion, can be used for detecting a cancer stem cell signature in the tumor sample and generating a cancer gene profile. More selective samples that are isolated from a heterogeneous patient sample such as, for example, by isolating tumorigenic cancer cells, laser capture microdissections, etc. can also be used. Alternatively the sample can permit the collection of cancer cells as well as normal cells for analysis so that the gene expression patterns for each sample can be determined and compared to a cancer stem cell gene signature to generate a cancer gene profile.

### Detection of Solid Tumor Stem Cell Cancer Markers and Cancer Stem Cell Signatures

In some embodiments, the present invention provides methods for detection of expression of stem cell cancer markers (e.g., breast cancer stem cell cancer markers). In some embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In some embodiments, the presence of a stem cell cancer marker is used to provide a prognosis to a subject. The information provided is also used to direct the course of treatment. For example, if a subject is found to have a marker indicative of a solid tumor stem cell (see, e.g. Tables 9A-9N2), additional therapies (e.g., hormonal or radiation therapies) can be started at an earlier point when they are more likely to be effective (e.g., before metastasis). In addition, if a subject is found to have a tumor that is not responsive to hormonal therapy, the expense and inconvenience of such therapies can be avoided.

The present invention is not limited to the markers described above. Any suitable marker that correlates with cancer or the progression of cancer can be utilized. Additional markers are also contemplated to be within the scope of the present invention. Any suitable method can be utilized to identify and characterize cancer markers suitable for use in the methods of the present invention, including but not limited to, those described in illustrative Example 1 below. For example, in some embodiments, markers identified as being up or down-regulated in solid tumor stem cells using the gene expression microarray methods of the present invention are further characterized using tissue microarray, immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of markers. The panel allows for the simultaneous analysis of multiple markers correlating with carcinogenesis and/or metastasis. Depending on the subject, panels can be analyzed alone or in combination in order to provide the best possible diagnosis and prognosis. Markers for inclusion on a panel are selected by screening for their predictive value using any suitable method, including but not limited to, those described in the illustrative examples below.

### Detection of RNA

In some embodiments, detection of solid tumor stem cell cancer markers (e.g., including but not limited to, those disclosed in Tables 9A-9N2) are detected by measuring the expression of corresponding mRNA in a tissue sample (e.g., breast cancer tissue). mRNA expression can be measured by any suitable method, including but not limited to, those disclosed below.

In some embodiments, RNA is detection by Northern blot analysis. Northern blot analysis involves the separation of RNA and hybridization of a complementary labeled probe.

In still further embodiments, RNA (or corresponding cDNA) is detected by hybridization to an oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

### Detection of Protein

In other embodiments, gene expression of stem cell cancer markers is detected by measuring the expression of the corresponding protein or polypeptide. Protein expression can be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In some embodiments, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

cDNA Microarray Technology

cDNA microarrays consist of multiple (usually thousands) of different cDNAs spotted (usually using a robotic spotting device) onto known locations on a solid support, such as a glass microscope slide. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. Full length cDNAs, expressed sequence tags (ESTs), or randomly chosen cDNAs from any library of interest can be chosen. ESTs are partially sequenced cDNAs as described, for example, in Hillier, et al., 1996, 6:807-828. Although some ESTs correspond to known genes, frequently very little or no information regarding any particular EST is available except for a small amount of 3′ and/or 5′ sequence and, possibly, the tissue of origin of the mRNA from which the EST was derived. As will be appreciated by one of ordinary skill in the art, in general the cDNAs contain sufficient sequence information to uniquely identify a gene within the human genome. Furthermore, in general the cDNAs are of sufficient length to hybridize, specifically or uniquely, to cDNA obtained from mRNA derived from a single gene under the hybridization conditions of the experiment.

In a typical microarray experiment, a microarray is hybridized with differentially labeled RNA, DNA, or cDNA populations derived from two different samples. Most commonly RNA (either total RNA or poly A+ RNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used. cDNA derived from one sample (representing, for example, a particular cell type, tissue type or growth condition) is labeled with one fluorophore while cDNA derived from a second sample (representing, for example, a different cell type, tissue type, or growth condition) is labeled with the second fluorophore. Similar amounts of labeled material from the two samples are cohybridized to the microarray. In the case of a microarray experiment in which the samples are labeled with Cy5 (which fluoresces red) and Cy3 (which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray.

Each microarray experiment can provide tens of thousands of data points, each representing the relative expression of a particular gene in the two samples. Appropriate organization and analysis of the data is of key importance, and various computer programs that incorporate standard statistical tools have been developed to facilitate data analysis. One basis for organizing gene expression data is to group genes with similar expression patterns together into clusters. A method for performing hierarchical cluster analysis and display of data derived from microarray experiments is described in Eisen et al., 1998, PNAS 95:14863-14868. As described therein, clustering can be combined with a graphical representation of the primary data in which each data point is represented with a color that quantitatively and qualitatively represents that data point. By converting the data from a large table of numbers into a visual format, this process facilitates an intuitive analysis of the data. Additional information and details regarding the mathematical tools and/or the clustering approach itself can be found, for example, in Sokal & Sneath, Principles of numerical taxonomy, xvi, 359, W. H. Freeman, San Francisco, 1963; Hartigan, Clustering algorithms, xiii, 351, Wiley, New York, 1975; Paull et al., 1989, J. Natl. Cancer Inst. 81:1088-92; Weinstein et al. 1992, Science 258:447-51; van Osdol et al., 1994, J. Natl. Cancer Inst. 86:1853-9; and Weinstein et al., 1997, Science, 275:343-9.

Further details of the experimental methods used in the present invention are found in the Examples. Additional information describing methods for fabricating and using microarrays is found in U.S. Pat. No. 5,807,522, which is herein incorporated by reference. Instructions for constructing microarray hardware (e.g., arrayers and scanners) using commercially available parts can be found at http://cmgm.stanford.edu/pbr-own/and in Cheung et al., 1999, Nat. Genet. Supplement 21:15-19, which are herein incorporated by reference. Additional discussions of microarray technology and protocols for preparing samples and performing microarray experiments are found in, for example, DNA arrays for analysis of gene expression, Methods Enzymol, 303:179-205, 1999; Fluorescence-based expression monitoring using microarrays, Methods Enzymol, 306: 3-18, 1999; and M. Schena (ed.), DNA Microarrays: A Practical Approach, Oxford University Press, Oxford, UK, 1999. Descriptions of how to use an arrayer and the associated software are found at http://cmgm.stanford.edu/pbrown/mguide/arrayerHTML/ArrayerDocs.html, which is herein incorporated by reference.

### Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or subjects can collect the sample themselves and directly send it to a profiling center. Where the sample comprises previously determined biological information, the information can be directly sent to the profiling service by the subject (e.g., an information card containing the information can be scanned by a computer and the data transmitted to a computer of the profiling center using an electronic communication system). Once received by the profiling service, the sample is processed and a profile is produced (e.g., expression data), specific for the diagnostic or prognostic information desired for the subject.

The profile data is then prepared in a format suitable for interpretation by a treating clinician. For example, rather than providing raw expression data (e.g. examining a number of the markers described in Tables 9A-9N2), the prepared format can represent a diagnosis or risk assessment for the subject, along with recommendations for particular treatment options. The data can be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject can chose further intervention or counseling based on the results. In some embodiments, the data is used for research use. For example, the data can be used to further optimize the inclusion or elimination of markers as useful indicators of a particular condition or stage of disease.

### Kits

In yet other embodiments, the present invention provides kits for the detection and characterization of cancer (e.g. for detecting one or more of the markers shown in Tables 9A-9N2, or for modulating the activity of a peptide expressed by one or more of markers shown in Tables 9A-9N2). In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In some embodiments, the kits contain all of the components necessary and/or sufficient to perform a detection assay, including all controls, directions for performing assays, and any necessary software for analysis and presentation of results.

Another aspect of the present invention comprises a kit to test for the presence of the polynucleotides or proteins, e.g. in a tissue sample or in a body fluid, of a cancer stem cell signature. The kit can comprise, for example, an antibody for detection of a polypeptide or a probe for detection of a polynucleotide. In addition, the kit can comprise a reference or control sample; instructions for processing samples, performing the test and interpreting the results; and buffers and other reagents necessary for performing the test. In certain embodiments the kit comprises a panel of antibodies for detecting expression of one or more of the proteins encoded by the genes of a cancer stem cell signature. In other embodiments the kit comprises pairs of primers for detecting expression of one or more of the genes of the cancer stem cell signature. In yet other embodiments the kit comprises a cDNA or oligonucleotide array for detecting expression of one or more of the genes of a cancer stem cell signature.

### In Vivo Imaging

In some embodiments, in vivo imaging techniques are used to visualize the expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the solid tumor stem cell cancer markers of the present invention (e.g., in breast cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancer stem cells can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 (1990) have described an optimized antibody-chelator for the radioimmunoscintographic imaging of tumors using Indium-111 as the label. Griffin et al., (J Clin One 9:631-640 (1991)) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 (1991)). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 (1980)) for In-111 and Tc-99m, and by Scheinberg et al. (Science 215:1511 (1982)). Other chelating agents can also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 (1982)) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A some method of labeling immunoglobulins with Tc-99m is that described by Wong et al. (Int. J. Appl. Radiat. Isot., 29:251 (1978)) for plasma protein, and recently applied successfully by Wong et al. (J. Nucl. Med., 23:229 (1981)) for labeling antibodies.

In the case of the radiometals conjugated to the specific antibody, it is likewise desirable to introduce as high a proportion of the radiolabel as possible into the antibody molecule without destroying its immunospecificity. A further improvement can be achieved by effecting radiolabeling in the presence of the specific stem cell cancer marker of the present invention, to insure that the antigen binding site on the antibody will be protected.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

### Antibodies and Antibody Fragments

The present invention provides isolated antibodies and antibody fragments (e.g., Fabs). In some embodiments, the present invention provides monoclonal antibodies or antibody fragments that specifically bind to an isolated polypeptide comprised of at least five, or at least 15 amino acid residues of the stem cell cancer markers described herein (e.g., as shown in Tables 9A-9N2). These antibodies or antibody fragments find use in the diagnostic, drug screening, and therapeutic methods described herein (e.g. to detect or modulate the activity of a stem cell cancer marker peptide).

An antibody, or antibody fragment, against a protein of the present invention can be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method can be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant can be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 (1975)). As a fusion promoter, for example, polyethylene glycol (PEG) or Sendai virus (HVJ) is used.

Examples of myeloma cells include NS-1, P3U1, SP2/0, AP-1 and the like. The proportion of the number of antibody producer cells (spleen cells) and the number of myeloma cells to be used is about 1:1 to about 20:1. PEG (e.g., PEG 1000-PEG 6000) can be added in concentration of about 10% to about 80%. Cell fusion can be carried out efficiently by incubating a mixture of both cells at about 20° C. to about 40° C. or about 30° C. to about 37° C. for about 1 minute to 10 minutes.

Various methods can be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, or 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., or 37° C. for about 5 days to 3 weeks, or 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies can be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. can be coupled to a hapten in a weight ratio of about 0.1 parts to about 20 parts, or about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant can be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a stem cell cancer marker of the present invention (further including a gene having a nucleotide sequence partly altered) can be used as the immunogen. Further, fragments of the protein can be used. Fragments can be obtained by any methods including, but not limited to expressing a fragment of the gene, enzymatic processing of the protein, chemical synthesis, and the like. The antibodies and antibody fragments can also be conjugated to therapeutic (e.g. cancer cell killing compounds). In this regard, the antibody directed toward one of the stem cell cancer markers is used to specifically deliver a therapeutic agent to a solid tumor cancer cell (e.g. to inhibit the proliferation of such sell or kill such a cell).

All of the various embodiments or options described herein can be combined in any and all variations.

### EXPERIMENTAL

The following examples are provided in order to demonstrate and further illustrate certain some embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and ° C. (degrees Centigrade).

### Example 1

### Identifying Stem Cell Cancer Markers

This Example describes how various stem cell cancer markers were identified using microarray screens. The results of these screens were processed and the names of the differentially expressed genes are reported in Tables 9A, 9B, 9C, 9D, 9E, 9F, 9G, 9H, 9I, 9J, 9K1, 9K2, 9L1, 9L2, 9M1, 9M2, 9N1 and 9N2; filed as Tables A, B, C, D, E, F, G, H, I, J, K1, K2, L1, L2, M1, M2, N1 and N2 in U.S. application Ser. No. 10/864,207, which is herein incorporated by reference.

In order to generate gene expression profiles, human breast tumorigenic cells that were initially isolated. A series of samples were accumulated from human breast tumors or normal tissues. These were generated as follows. Three passaged breast tumors—breast tumor cells from patient 1, 2, 3 were engrafted on mice. Each tumor was engrafted on three mice to make the triplicate tumors. The breast tumorigenic cells were then isolated from these tumors. Two or three unpassaged breast tumors from three patients SUM, PE13, PE15 were labeled and sorted into tumorigenic cells (TG) or non-tumorigenic cells (NTG). Both PE15-TG and PE15-NTG were triplicate. Two or three normal breast samples were from breast reduction patients. Breast epithelial cells (Breast) were isolated with flow cytometry and used for microarray. Two or three normal colon samples were collected freshly from colon patients. Colon epithelial cells (Colon) were isolated with flow cytometry and used for microarray. Probes were made from the isolated cells types for use in the microarray analysis.

In order to perform the various microarray screens Affymetrix HG-U133 gene chips were used. The normalized gene expression intensity was used to generate the data that was collected in a number of large tables. Gene expression was determined using standard techniques known in the art for microarray analysis, such as those found in “GeneChip® Expression Analysis” (Affymetrix Technical Manual Rev. 5, 2004). The results in these tables was processed and used to generate Tables 9A, 9B, 9C, 9D, 9E, 9F, 9G, 9H, 9I, 9J, 9K1, 9K2, 9L1, 9L2, 9M1, 9M2, 9N1 and 9N2, which present the names of the genes found to be differentially expressed, including genes found to be down regulated in UPTG versus normal breast tissue, genes found to be up regulated in UPTG versus normal breast tissue, genes found to be down regulated in UPTG versus normal colon tissue, and genes found to be up regulated in UPTG versus normal colon tissue. In the tables, the column headers refer to the gene's name or samples name and array numbers.

### Example 2

### Identification of Cancer Stem Cell Gene Signatures that Predict Clinical Outcome

This example describes the identification of a cancer stem cell gene signature useful to diagnose, predict clinical outcome of a cancer including metastasis and overall survival, as well as for use in clinical trials for achieving stratification of patients and for testing of a potential therapeutic for treating cancer and for guiding cancer therapy. The cancers for which a cancer stem cell gene signature can be used to predict clinical outcome include for example, breast cancer, colon and rectum cancer, pancreas, lung and bronchus, urinary bladder, kidney, head and neck cancer and additional cancers: as set forth in more detail above. Furthermore, use of these cancer stem cell gene signatures to classify tumor samples into low and high risk for the purpose of prognosis and therapy selection is provided.

### Cancer Stem Cell Gene Signatures 1 & 2 Generated by Expression Level and P Value

The genome-wide analysis of the present invention identified cancer stem cell gene signatures by determining gene expression levels in tumor stem cells compared to a non-tumorigenic tissue, such as normal breast epithelium. These gene expression levels were then further sorted and the resulting tumor stem cell gene signatures tested for the ability to predict clinical outcome including metastasis and death. To generate a tumor stem cell gene signature from the microarray comparison of cancer stem cells gene expression versus normal breast tissue, genes with two-fold reduced or two-fold elevated expression in cancer stem cells were identified. These genes were then further filtered by the P value of a t-test of 0.005 or 0.012 to generate two cancer stem cell gene signatures (gene signatures 1 & 2) comprising 215 and 367 genes, respectively (Tables 1A-2A above).

To assess the ability of these cancer stem cell gene signatures to predict metastasis and death a number of different independent cancer patient populations were used (the cancer patient datasets) including: 295 consecutive early breast cancer patients from the Netherlands Cancer Institute (NKI) (van de Vijver et al., 2002, N. Eng. J. Med. 347:1999), and 286 lymph node negative breast cancer patients from the Erasmus Medical Center (GEO accession GSE2034) (Wang et al., 2005, Lancet 365:671), 159 breast cancer patients (GEO accession GSE1456) (Pawitan et al., 2005, Breast Cancer Res. 7:R953); 236 primary breast cancers (GEO accession GSE 3494) (Miller et al., 2005, PNAS 102:13550); and 189 invasive breast carcinomas (GEO accession GSE2990) (Sotiriou et al., 2006, Cancer Inst. 98: 262-72).

Cancer stem cell gene signature 1 comprises 215 genes selected from two-fold decreased and two-fold elevated expression in tumor stem cells versus normal breast tissue with a t-test P value of 0.005 across samples (Table 1A). Of the 215 genes that comprise signature one, 142 (Table 1B) are shared with the array used to analyze expression of the 295 patients from the Netherlands Cancer Institute (the NKI array). Correlation and Cox proportional hazard survival analysis of microarray data from these 295 patients revealed cancer stem cell gene signature 1 as significantly predictive of metastasis with a univariate hazard ratio of 1.21 per 0.1 correlation (P=2.5×10⁻⁵) and significantly predictive of death with a univariate hazard ratio of 1.30 per 0.1 correlation (P=1.3×10⁻⁶) (Table 10). When the 295 patents from the Netherlands study were divided into patient populations based on estrogen receptor expression (ER) status, 226 were ER positive and 69 were ER negative. Correlation and Cox proportional hazard survival analysis revealed cancer stem cell gene signature 1 was significantly predicative of metastasis and death in the ER+ patient population with a univariate hazard ratio of 1.12 per 0.1 correlation (P=8.5×10⁻⁵) and 1.36 per 0.1 correlation (P=8.2×10⁻⁶), respectively (Table 10).

TABLE 10
Cancer Stem Cell Gene Signature 1 Predictive of Clinical
Outcome in NKI Dataset

| | Death | | Metastasis | |
|---|---|---|---|---|
| | | Hazard ratio | | Hazard ratio |
| | P value | per 0.1 | P value | per 0.1 |
| NKI | 1.3e−6 | 1.30 | 2.5e−5 | 1.21 |
| NKI_ER+ (226) | 8.2e−6 | 1.36 | 8.5e−5 | 1.12 |
| NKI_ER− (69) | 0.45 | 1.08 | 0.21 | 1.11 |
| NKI_LN− (151) | 0.0011 | 1.28 | 0.003 | 1.21 |
| NKI_LN_1 (58) | 0.015 | 1.41 | 0.007 | 1.47 |
| NKI_LN+ (144) | 0.00049 | 1.21 | 0.0029 | 1.21 |

Kaplan-Meier survival analysis was used to compare the occurrence of metastasis and death between patients with correlations greater than average and those with correlations less than or equal to average (FIG. 1). A logrank test revealed a significant difference between these two groups for both metastasis (P=0.0001) and overall survival (P<0.0001).

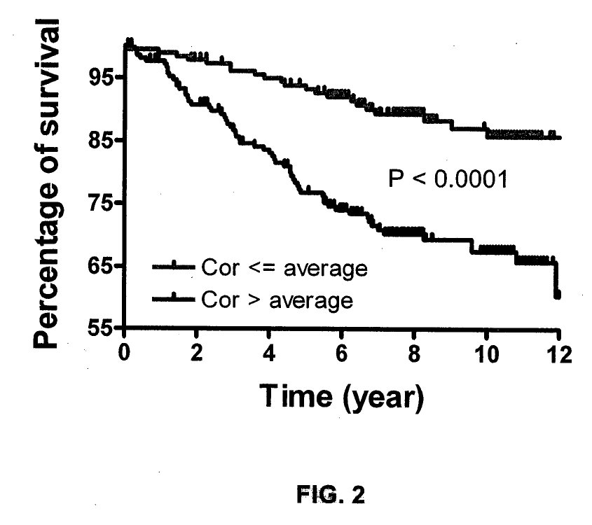

All 215 genes of cancer gene signature 1 were present in the GSE1456 and GSE3494 patient datasets. Correlation and Cox proportional hazard survival analysis of microarray data from these breast cancer patients revealed cancer stem cell gene signature 1 as significantly predictive of death with a univariate hazard ratio of 1.23 per 0.1 correlation (P=3.2×10⁻⁷). Kaplan-Meier survival analysis was used to compare the occurrence of death between patients with correlations greater than average and those with correlations less than or equal to average (FIG. 2). A logrank test revealed a significant difference between these two groups for overall survival (P<0.0001).

Of the 215 genes that comprise signature one, 126 (Table 1C) are shared with the array used to analyze expression of the 286 patients from the Erasmus Medical Center (GSE2034). The analysis showed that the risk of metastasis was significantly higher among patients with an expression profile highly correlated with this gene signature (Cor>average) than among those with an expression profile not correlated with the gene signature (Cor<=average, Table 11, P=0.0002 by chi-square test).

TABLE 11
Cancer Stem Cell Gene Signature 1 Highly Correlated With the Risk of
Metastasis in GSE2034 dataset

| | Relapse free | Metastasis |
|---|---|---|
| Cor >= average (0.206) | 92 | 77 |
| Cor < average | 89 | 29 |

Cancer stem cell gene signature 2 comprises 367 genes selected from two-fold decreased and two-fold elevated expression in tumor stem cells versus normal breast tissue with a t-test P value of 0.012 across samples (Table 2A). Of the 367 genes that comprise signature one, 248 (Table 2B) are shared with the array used to analyze expression of the 295 patients from the Netherlands Cancer Institute (the 295 NKI array). Correlation and Cox proportional hazard survival analysis of microarray data from these 295 patients revealed cancer stem cell gene signature 2 as significantly predictive of metastasis with a univariate hazard ratio of 1.25 per 0.1 correlation (P=9.1×10⁻⁶) and significantly predictive of death with a univariate hazard ratio of 1.33 per 0.1 correlation (P=1.4×10⁻⁶) (Table 12). When the 295 patents from the Netherlands study were divided into patient populations based on estrogen receptor expression (ER) status, 226 were ER positive and 69 were ER negative. Correlation and Cox proportional hazard survival analysis revealed cancer stem cell gene signature 2 was significantly predicative of metastasis and death in the ER+ patient population with a univariate hazard ratio of 1.13 per 0.1 correlation (P=4.6×10⁻⁵) and 1.40 per 0.1 correlation (P=1.1×10⁻⁵), respectively (Table 12).

TABLE 12
Cancer Stem Cell Gene Signature 2 Predictive of Clinical Outcome in NKI
Dataset

| | Death | | Metastasis | |
|---|---|---|---|---|
| | | Hazard ratio per | | Hazard ratio |
| | P value | 0.1 | P value | per 0.1 |
| NKI ( | 1.4e−6 | 1.33 | 9.1e−6 | 1.25 |
| NKI_ER+ (226) | 1.1e−5 | 1.40 | 4.6e−5 | 1.13 |
| NKI_ER− (69) | 0.24 | 1.14 | 0.3 | 1.13 |
| NKI_LN− (151) | 0.0015 | 1.31 | 0.0021 | 1.21 |
| NKI_LN_1 (58) | 0.012 | 1.54 | 0.004 | 1.68 |
| NKI_LN+ (144) | 0.00033 | 1.36 | 0.0016 | 1.21 |

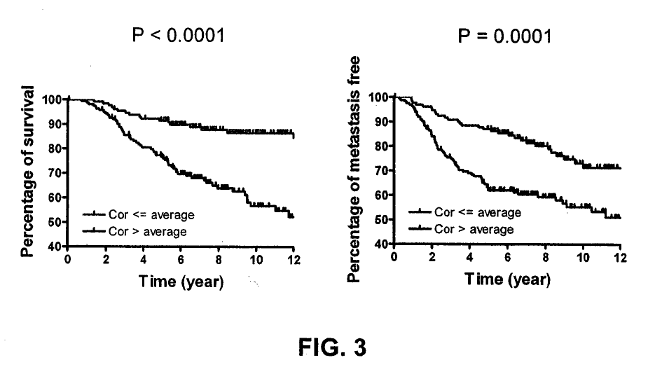

Kaplan-Meier survival analysis was used to compare the occurrence of metastasis and death between patients with correlations greater than average and those with correlations less than or equal to average (FIG. 3). A logrank test revealed a significant difference between these two groups for both metastasis (P=0.0001) and overall survival (P<0.0001).

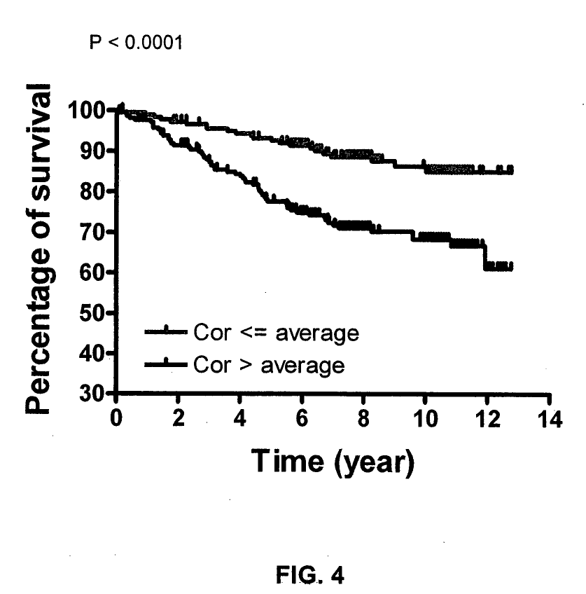

All 367 genes of cancer gene signature 2 were present in the GSE1456 and GSE3494 patient datasets. Correlation and Cox proportional hazard survival analysis of microarray data from these breast cancer patients revealed cancer stem cell gene signature 2 as significantly predictive of death with a univariate hazard ratio of 1.22 per 0.1 correlation (P=5.4×10⁻⁷). Kaplan-Meier survival analysis was used to compare the occurrence of death between patients with correlations greater than average and those with correlations less than or equal to average (FIG. 4). A logrank test revealed a significant difference between these two groups for overall survival (P<0.0001).

Of the 367 genes that comprise signature 2, 226 (Table 2C) are shared with the array used to analyze expression of the 286 patients from the Erasmus Medical Center (GSE2034). The analysis showed that the risk of metastasis was significantly higher among patients with an expression profile highly correlated with this gene signature (Cor>average) than among those with an expression profile not correlated with the gene signature (Cor<=average, Table 13, P<0.0001 by chi-square test).

TABLE 13
Cancer Stem Cell Gene Signature 2 Highly Correlated With the Risk of
Metastasis in GSE2034 dataset

| | Relapse free | Metastasis |
|---|---|---|
| Cor >= average (0.22) | 84 | 77 |
| Cor < average | 96 | 29 |

### Cancer Stem Cell Gene Signatures 3 Generated by Expression Level and P Value

The genome-wide analysis of the present invention identified cancer stem cell gene signatures by determining gene expression levels in tumor stem cells compared to a non-tumorigenic tissue. These gene expression levels were then further sorted and the resulting tumor stem cell gene signatures tested for the ability to predict clinical outcome including metastasis and death. The genes differentially expressed in colon tumor stem cells versus nontumorigenic colon cells are divided into different tumor stem cell gene signatures based on the fold expression change. Specifically, the microarray analysis of the invention was used to identify genes with three-fold reduced and three-fold elevated expression in tumorigenic colon cells versus non-tumorigenic colon cells. This tumor stem cell gene signature was then further filtered by the P value of a t-test 0.04 between the tumorigenic and non-tumorigenic samples to generate cancer stem cell gene signature 3 comprising 315 genes (Tables 3A).

To assess the ability of this cancer stem cell gene signature to predict metastasis and death a number of different independent cancer patient populations were used (the cancer patient datasets) including: 159 breast cancer patients (GEO accession GSE1456) (Pawitan et al., 2005, Breast Cancer Res. 7:R953); and 236 primary breast cancers (GEO accession GSE 3494) (Miller et al., 2005, PNAS 102:13550).

All 315 genes of cancer gene signature 3 were present in the GSE1456 and GSE3494 patient datasets. Correlation and Cox proportional hazard survival analysis of microarray data from the 159 patients in the GSE 1456 dataset revealed cancer stem cell gene signature 3 as significantly predictive of relapse with a univariate hazard ratio of 1.45 per 0.1 correlation (P=4.5×10⁻⁴) and significantly predictive of death with a univariate hazard ratio of 1.47 per 0.1 correlation (P=0.002) (Table 14). When the 159 patents were divided into patient populations based on estrogen receptor expression (ER) status, 132 were ER positive and 27 were ER negative. Correlation and Cox proportional hazard survival analysis revealed cancer stem cell gene signature 3 was significantly predicative of relapse and death in the ER+ patient population with a univariate hazard ratio of 1.45 per 0.1 correlation (P=0.0013) and 1.53 per 0.1 correlation (P=0.0029), respectively (Table 14).

TABLE 14
Cancer Stem Cell Gene Signature 3 Predictive of Clinical Outcome in
GSE1456 Dataset

| | Death | | Relapse | |
|---|---|---|---|---|
| | | Hazard ratio | | Hazard ratio per |
| | P value | per 0.1 | P value | 0.1 |
| GSE1456 (159 | 0.002 | 1.47 | 4.5e−4 | 1.45 |
| patients) | | | | |
| GSE1456 ER+ | 0.0029 | 1.53 | 0.0013 | 1.45 |
| (132) | | | | |
| GSE1456_ER− | 0.87 | 1.05 | 0.61 | 1.15 |
| (27) | | | | |

Correlation and Cox proportional hazard survival analysis of microarray data from the 236 patients in the GSE 3494 dataset revealed cancer stem cell gene signature 3 is also predictive in that dataset (Table 15).

TABLE 15
Cancer Stem Cell Gene Signature 3 Predictive of Clinical Outcome in
GSE3494 Dataset

Death

| | P value | Hazard ratio per 0.1 |
|---|---|---|
| GSE3494 | 0.028 | 1.17 |
| GSE3494 ER+ (201) | 0.045 | 1.17 |
| GSE3494_ER− (31) | 0.3 | 1.30 |

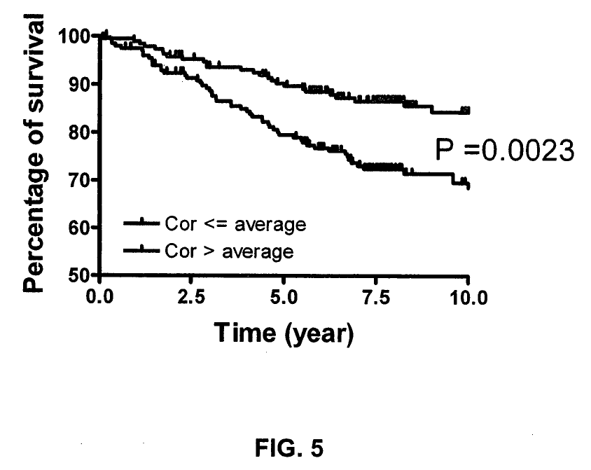

Correlation and Cox proportional hazard survival analysis of microarray data from the combined patients in the GSE 1456 and 3494 dataset revealed cancer stem cell gene signature 3 as predictive of death with a univariate hazard ratio of 1.25 per 0.1 correlation (P=6.1×10⁻⁴). Kaplan-Meier survival analysis was used to compare the occurrence of death between patients with correlations greater than average and those with correlations less than or equal to average (FIG. 5). A logrank test revealed a significant difference between these two groups for overall survival (P=0.0023).

### Cancer Stem Cell Gene Signatures 4 Through 8

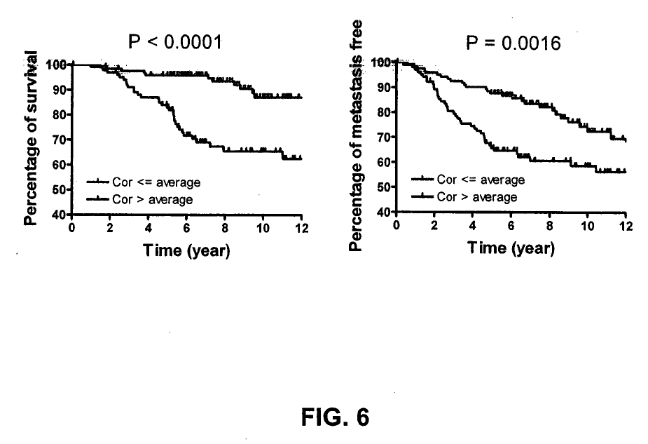

Of the 52 genes that comprise signature 4, 39 (Table 5B) are shared with the array used to analyze expression of the 295 patients from the Netherlands Cancer Institute (the NKI array). Correlation and Cox proportional hazard survival analysis of microarray data from these 295 patients revealed cancer stem cell gene signature 4 as significantly predictive of metastasis with a univariate hazard ratio of 1.13 per 0.1 correlation (P=2.6×10⁻⁶) and significantly predictive of death with a univariate hazard ratio of 1.21 per 0.1 correlation (P=3.6×10⁻⁹) (Table 16). When the 295 patents from the Netherlands study were divided into patient populations based on estrogen receptor expression (ER) status, 226 were ER positive and 69 were ER negative. Correlation and Cox proportional hazard survival analysis revealed cancer stem cell gene signature 4 was significantly predicative of metastasis and death in the ER+ patient population with a univariate hazard ratio of 1.12 per 0.1 correlation (P=8.5×10⁻⁵) and 1.19 per 0.1 correlation (P=4.9×10⁻⁶), respectively (Table 16). Kaplan-Meier survival analysis was used to compare the occurrence of metastasis and death between patients with correlations greater than average and those with correlations less than or equal to average (FIG. 6). A logrank test revealed a significant difference between these two groups for both metastasis (P=0.0016) and overall survival (P<0.0001).

TABLE 16
Cancer Stem Cell Gene Signature 4 Predictive of
Clinical Outcome in NKI Dataset

| | Death | | Metastasis | |
|---|---|---|---|---|
| | | Hazard | | Hazard |
| | P value | ratio per 0.1 | P value | ratio per 0.1 |
| NKI | 3.6e−9 | 1.21 | 2.6e−6 | 1.13 |
| NKI_ER+ | 4.9e−6 | 1.19 | 8.5e−5 | 1.12 |
| (226) | | | | |
| NKI_ER− | 0.1 | 1.15 | 0.21 | 1.11 |
| (69) | | | | |
| NKI_LN− | 9e−6 | 1.21 | 2.9e−4 | 1.13 |
| (151) | | | | |
| NKI_LN_1 | 0.011 | 1.31 | 0.0093 | 1.29 |
| (58) | | | | |
| NKI_LN+ | 1e−4 | 1.21 | 2.7e−3 | 1.11 |
| (144) | | | | |

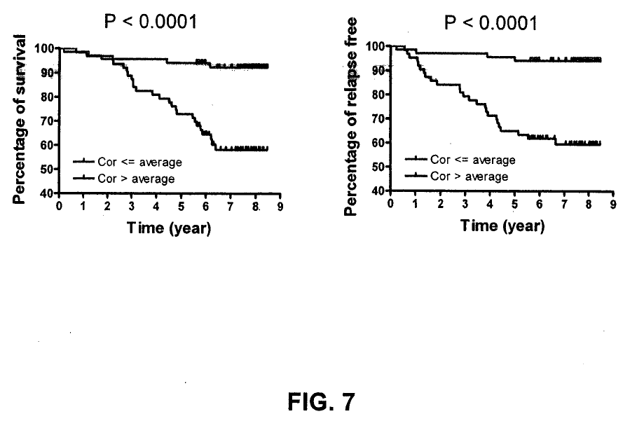

All 52 genes of cancer gene signature 4 were present in the GSE1456 and GSE3494 patient datasets. Correlation and Cox proportional hazard survival analysis of microarray data from the 159 patients in the GSE 1456 dataset revealed cancer stem cell gene signature 4 as significantly predictive of relapse with a univariate hazard ratio of 1.16 per 0.1 correlation (P=1.0×10⁻⁵) and significantly predictive of death with a univariate hazard ratio of 1.13 per 0.1 correlation (P=1.3×10⁻⁴) (Table 17). When the 159 patents were divided into patient populations based on estrogen receptor expression (ER) status, 132 were ER positive and 27 were ER negative. Correlation and Cox proportional hazard survival analysis revealed cancer stem cell gene signature 4 was significantly predicative of relapse and death in the ER+ patient population with a univariate hazard ratio of 1.18 per 0.1 correlation (P=2.7×10⁻⁵) and 1.15 per 0.1 correlation (P=1.9×10⁻⁴), respectively (Table 17). Kaplan-Meier survival analysis was used to compare the occurrence of relapse and death between patients with correlations greater than average and those with correlations less than or equal to average (FIG. 7). A logrank test revealed a significant difference between these two groups for both (P<0.0001).

TABLE 17
Cancer Stem Cell Gene Signature 4 Predictive
of Clinical Outcome in GSE1456 Dataset

| | Death | | Relapse | |
|---|---|---|---|---|
| | | Hazard | | Hazard |
| | P value | ratio per 0.1 | P value | ratio per 0.1 |
| GSE1456 (159 patients) | 1.3e−4 | 1.13 | 1e−5 | 1.16 |
| GSE1456 ER+ (132) | 1.9e−4 | 1.15 | 2.7e−5 | 1.18 |
| GSE1456_ER− (27) | 0.62 | 1.07 | 0.65 | 1.06 |

Correlation and Cox proportional hazard survival analysis of microarray data from the 236 patients in the GSE 3494 dataset revealed cancer stem cell gene signature 4 as significantly predictive of death with a univariate hazard ratio of 1.15 per 0.1 correlation (P=5.1×10⁻⁷) (Table 18). When the 236 patients were divided into patient populations based on estrogen receptor expression (ER) status, 201 were ER positive and 31 were ER negative. Correlation and Cox proportional hazard survival analysis revealed cancer stem cell gene signature 4 was significantly predicative of death in the ER+ patient population with a univariate hazard ratio of 1.16 per 0.1 correlation (P=1.9×10⁻⁷) (Table 18).

TABLE 18
Cancer Stem Cell Gene Signature 4 Predictive
of Clinical Outcome in GSE3494 Dataset

Death

| | | Hazard |
|---|---|---|
| | P value | ratio per 0.1 |
| GSE3494 | 5.1e−7 | 1.15 |
| GSE3494 ER+ (201) | 1.9e−7 | 1.16 |
| GSE3494 ER− (31) | 0.24 | 1.54 |
| GSE3494 LN+ (78) | 0.00048 | 1.16 |
| GSE3494 LN− (149) | 0.011 | 1.11 |

Of the 52 genes that comprise signature 4, 43 (Table 4C) are shared with the array used to analyze expression of the 286 patients from the Erasmus Medical Center (GSE2034). The analysis showed that the risk of metastasis was significantly higher among patients with an expression profile highly correlated with this gene signature (Cor>average) than among those with an expression profile not correlated with the gene signature (Cor<=average, Table 19, P<0.0001 by chi-square test). Similar results were obtained from the ER+ patients (Table 20, P<0.0001 by chi-square test).

TABLE 19
Cancer Stem Cell Gene Signature 4 Highly Correlated
With the Risk of Metastasis in GSE2034 dataset

| | Relapse free | Metastasis |
|---|---|---|
| Cor >= average (0.12) | 80 | 73 |
| Cor < average | 100 | 33 |

TABLE 20
Cancer Stem Cell Gene Signature 4 Highly Correlated With the
Risk of Metastasis in ER+ patients of GSE2034 dataset

| | Relapse free | Metastasis |
|---|---|---|
| Cor >= average (0.12) | 35 | 48 |
| Cor < average | 94 | 29 |

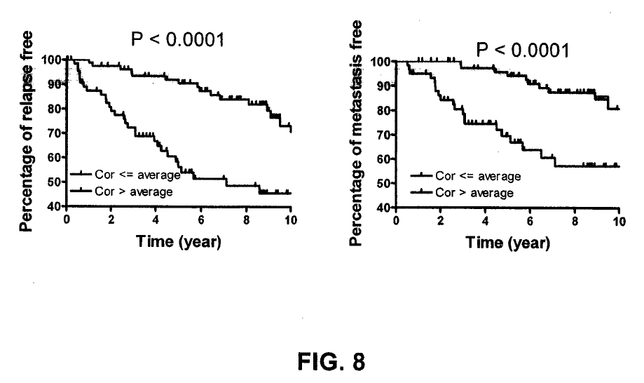

Of the 52 genes that comprise signature 4, 43 (Table 4C) are shared with the array used to analyze expression of the 189 patients from GSE 2990 dataset. Correlation and Cox proportional hazard survival analysis revealed cancer stem cell gene signature 4 as significantly predictive of metastasis with a univariate hazard ratio of 1.09 per 0.1 correlation (P=2.9×10⁻³) and of relapse with a univariate hazard ratio of 1.08 per 0.1 correlation (P=5.3×10⁻⁴) (Table 21). When the patients were divided into populations based on estrogen receptor expression (ER) status, correlation and Cox proportional hazard survival analysis revealed cancer stem cell gene signature 4 was significantly predicative of metastasis and relapse in the ER+ patient population with a univariate hazard ratio of 1.15 per 0.1 correlation (P=4.3×10⁻⁵) and (P=1.9×10⁻⁴), respectively (Table 21). Kaplan-Meier survival analysis was used to compare the occurrence of relapse and metastasis between patients with correlations greater than average and those with correlations less than or equal to average (FIG. 8). A logrank test revealed a significant difference between these two groups for both (P<0.0001).

TABLE 21
Cancer Stem Cell Gene Signature 4 Predictive
of Clinical Outcome in GSE2990 dataset

| | Relapse | | Distance metastasis | |
|---|---|---|---|---|
| | | Hazard | | Hazard |
| | | ratio | | ratio |
| | P value | per 0.1 | P value | per 0.1 |
| GSE2990 | 5.3e−4 | 1.08 | 2.9e−3 | 1.09 |
| ER+ | 1.9e−4 | 1.15 | 4.3e-5 | 1.15 |
| | (147) | | (139) | |
| ER− | 0.62 (34) | 1.07 | 0.33 (34) | 0.94 |
| Node+ | 0.035 | 1.24 | 0.044 | 1.23 |
| Node− | 0.0053 | 1.07 | 0.017 | 1.08 |

Correlation and Cox proportional hazard survival analysis of microarray data from the patients in the combined GSE 1456 and GSE 3494 datasets revealed cancer stem cell gene signatures 4-8 as significantly predictive of death (Table 22). When the patients were divided into patient populations based on estrogen receptor expression (ER) status, correlation and Cox proportional hazard survival analysis revealed cancer stem cell gene signatures 4-8 were significantly predicative of death in the ER+ patient population (Table 22).

TABLE 22
Cancer Stem Cell Gene Signatures 4-8 Predictive of Clinical
Outcome in combined GSE1456 and GSE3494 datasets

Death

| | | | Hazard |
|---|---|---|---|
| | | P value | ratio per 0.1 |
| 26 Gene list (26) | Total (391) | 7.9e−11 | 1.16 |
| | ER− (58) | 0.12 | 1.18 |
| | ER+ (333) | 9.3e−11 | 1.17 |
| 34 gene list (34) | Total | 1.2e−10 | 1.15 |
| | ER− | 0.12 | 1.19 |
| | ER+ | 1.2e−10 | 1.16 |
| 52 gene list (52) | Total | 2.3e−10 | 1.14 |
| | ER− | 0.1 | 1.19 |
| | ER+ | 1.1e−10 | 1.16 |
| 16 gene list (16) | Total | 1.8e−10 | 1.13 |
| | ER− | 0.18 | 1.14 |
| | ER+ | 1.2e−10 | 1.14 |
| 74 gene list (74) | Total | 3.4e−10 | 1.15 |
| | ER− | 0.2 | 1.12 |
| | ER+ | 2.5e-10 | 1.17 |

Correlation and Cox proportional hazard survival analysis of microarray data from the patients in the NKI dataset revealed cancer stem cell gene signatures 4-8 as significantly predictive of death and metastasis (Table 23). When the patients were divided into patient populations based on estrogen receptor expression (ER) status, correlation and Cox proportional hazard survival analysis revealed cancer stem cell gene signatures 4-8 were significantly predicative of death and metastasis in the ER+ patient population (Table 23).

TABLE 23
Cancer Stem Cell Gene Signatures 4-8 Predictive
of Clinical Outcome in NKI Dataset

| | | Death | | Metastasis | |
|---|---|---|---|---|---|
| | | | Hazard | | Hazard |
| | | | ratio | | ratio |
| | | P value | per 0.1 | P value | per 0.1 |
| 26 (22) | NKI (295) | 1.1e−9 | 1.24 | 1.3e−7 | 1.16 |
| | NKI_ER+ (226) | 5.8e−7 | 1.23 | 5.7e−6 | 1.15 |
| | NKI_ER− (69) | 0.043 | 1.20 | 0.051 | 1.21 |
| 34 (29) | NKI | 2.2e−9 | 1.21 | 6.7e−7 | 1.14 |
| | NKI_ER+ | 1.4e−6 | 1.23 | 2.2e−5 | 1.15 |
| | NKI_ER− | 0.067 | 1.16 | 0.12 | 1.21 |
| 52 (41) | NKI | 3.6e−9 | 1.21 | 2.6e−6 | 1.13 |
| | NKI_ER+ | 4.9e−6 | 1.19 | 8.5e−5 | 1.12 |
| | NKI_ER− | 0.1 | 1.15 | 0.21 | 1.11 |
| 16 (13) | NKI | 2.1e−7 | 1.12 | 1.5e−5 | 1.08 |
| | NKI_ER+ | 3e−5 | 1.12 | 1.5e−4 | 1.08 |
| | NKI_ER− | 0.23 | 1.05 | 0.39 | 1.04 |
| 74 (58) | NKI | 1.4e−8 | 1.21 | 2.8e−6 | 1.14 |
| | NKI_ER+ | 7.4e−6 | 1.20 | 4.7e−5 | 1.13 |
| | NKI_ER− | 0.16 | 1.12 | 0.3 | 1.09 |

Correlation and Cox proportional hazard survival analysis of microarray data from the patients in the GSE 2990 dataset revealed cancer stem cell gene signatures 4-8 as significantly predictive of relapse and metastasis (Table 24). When the patients were divided into patient populations based on estrogen receptor expression (ER) status, correlation and Cox proportional hazard survival analysis revealed cancer stem cell gene signatures 4-8 were significantly predicative of relapse and metastasis in the ER+ patient population (Table 24).

TABLE 24
Cancer Stem Cell Gene Signatures 4-8 Predictive
of Clinical Outcome in GSE2990 Dataset

| | | Relapse | | Distance metastasis | |
|---|---|---|---|---|---|
| | | | Hazard | | Hazard |
| | | | ratio | | ratio |
| | | P value | per 0.1 | P value | per 0.1 |
| 26 (22) | Total (189) | 7e−4 | 1.08 | 1.2e−3 | 1.10 |
| | ER+ (147, | 3.6e−5 | 1.12 | 1.7e−5 | 1.17 |
| | 139) | | | | |
| | ER− (34) | 0.91 | 0.994 | 0.33 | 0.937 |
| 34 (30) | total | 3.6e−4 | 1.08 | 8.6e−4 | 1.10 |
| | ER+ | 3e−5 | 1.12 | 1.1e−5 | 1.17 |
| | ER− | 0.91 | 1.01 | 0.33 | 0.94 |
| 52 (43) | total | 5.3e−4 | 1.08 | 2.9e−3 | 1.09 |
| | ER+ | 1.9e−4 | 1.15 | 4.3e−5 | 1.15 |
| | ER− | 0.94 | 1.07 | 0.33 | 0.94 |
| 16 (16) | total | 2.3e−3 | 1.06 | 4.6e−3 | 1.07 |
| | ER+ | 5.3e−4 | 1.08 | 1.7e−4 | 1.12 |
| | ER− | 0.86 | 1.01 | 0.45 | 0.96 |
| 74 (57) | total | 1.1e−3 | 1.08 | 1.2e−3 | 1.11 |
| | ER+ | 1.6e−4 | 1.11 | 2.4e−5 | 1.17 |
| | ER− | 0.82 | 1.01 | 0.38 | .94 |

Of the 286 patients from the Erasmus Medical Center (GSE2034), the risk of metastasis was significantly higher among patients with an expression profile highly correlated with this gene signature (Cor>average) than among those with an expression profile not correlated with the gene signature (Cor<=average, Table 25, P=0.0158 by chi-square test). Similar results were obtained from the ER+ patients (Table 26, P=0.0004 by chi-square test).

TABLE 25
Cancer Stem Cell Gene Signature 6 Highly Correlated
With the Risk of Metastasis in GSE2034 dataset

| | Relapse free | Metastasis |
|---|---|---|
| Cor >= average | 89 | 68 |
| Cor < average | 91 | 38 |

TABLE 26
Cancer Stem Cell Gene Signature 6 Highly Correlated With the
Risk of Metastasis in ER+ patients of GSE2034 dataset

| | Relapse free | Metastasis |
|---|---|---|
| Cor >= average | 46 | 47 |
| Cor < average | 83 | 30 |

Of the 286 patients from the Erasmus Medical Center (GSE2034), the risk of metastasis was significantly higher among patients with an expression profile highly correlated with this gene signature (Cor>average) than among those with an expression profile not correlated with the gene signature (Cor<=average, Table 27, P=0.0002 by chi-square test). Similar results were obtained from the ER+ patients (Table 28, P<0.0001 by chi-square test).

TABLE 27
Cancer Stem Cell Gene Signature 5 Highly Correlated
With the Risk of Metastasis in GSE2034 dataset

| | Relapse free | Metastasis |
|---|---|---|
| Cor >= average | 85 | 74 |
| Cor < average | 95 | 32 |

TABLE 28
Cancer Stem Cell Gene Signature 5 Highly Correlated With the
Risk of Metastasis in ER+ patients of GSE2034 dataset

| | Relapse free | Metastasis |
|---|---|---|
| Cor >= average (0.12) | 43 | 50 |
| Cor < average | 86 | 27 |

Of the 286 patients from the Erasmus Medical Center (GSE2034), the risk of metastasis was significantly higher among patients with an expression profile highly correlated with this gene signature (Cor>average) than among those with an expression profile not correlated with the gene signature (Cor<=average, Table 29, P=0.0045 by chi-square test). Similar results were obtained from the ER+ patients (Table 30, P=0.0005 by chi-square test).

TABLE 29
Cancer Stem Cell Gene Signature 8 Highly Correlated
With the Risk of Metastasis in GSE2034 dataset

| | Relapse free | Metastasis |
|---|---|---|
| Cor >= average (0.007) | 86 | 69 |
| Cor < average | 94 | 37 |

TABLE 30
Cancer Stem Cell Gene Signature 8 Highly Correlated
With the Risk of Metastasis in GSE2034 dataset

| | Relapse free | Metastasis |
|---|---|---|
| Cor >= average (0.12) | 39 | 42 |
| Cor < average | 90 | 35 |

Of the 286 patients from the Erasmus Medical Center (GSE2034), the risk of metastasis was higher among patients with an expression profile highly correlated with this gene signature (Cor>average) than among those with an expression profile not correlated with the gene signature (Cor<=average, Table 31, P=0.077 by chi-square test). Similar results were obtained from the ER+ patients (Table 32, P=0.0075 by chi-square test).

TABLE 31
Cancer Stem Cell Gene Signature 7 Correlated With
the Risk of Metastasis in GSE2034 dataset

| | Relapse free | Metastasis |
|---|---|---|
| Cor >= average (0.1346) | 91 | 65 |
| Cor < average | 89 | 41 |

TABLE 32
Cancer Stem Cell Gene Signature 7 Highly Correlated With the
Risk of Metastasis in ER+ patients of GSE2034 dataset

| | Relapse free | Metastasis |
|---|---|---|
| Cor >= average (0.1346) | 49 | 44 |
| Cor < average | 80 | 33 |

Table 33 provides a summary of the results for the GSE 2034 dataset.

TABLE 33
Summarized Chi-Square P Values for Gene Signature 4-8 in GSE2034
Dataset and in ER+ patients of GSE2034 Dateset

| | | P value (Chi-square test) |
|---|---|---|
| 26 (22) | Total (286) | 0.016 |
| | ER+ (206) | 0.0004 |
| 34 (30) | Total | 0.0002 |
| | ER+ | <0.0001 |
| 52 (43) | Total | <0.0001 |
| | ER+ | <0.0001 |
| 16 (16) | Total | 0.0045 |
| | ER+ | 0.0005 |
| 74 (57) | Total | 0.077 |
| | ER+ | 0.0075 |

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications and patents mentioned in the above specification are herein incorporated by reference in their entirety.

1. A method of classifying a cancer, the method comprising:
determining expression levels of one or more genes in a cancer sample in comparison to expression levels of the gene(s) in a reference sample, wherein the gene(s) are selected from a cancer stem cell gene signature;
comparing the expression levels of the gene(s) in the cancer sample in (a) to the expression levels of the gene(s) comprising the cancer stem cell gene signature; and,
classifying the cancer to either a high risk or low risk group based on the comparison in (b).

2. The method of claim 1 wherein determining the expression levels of one or more genes selected from the cancer stem cell gene signature is by measuring expression of the corresponding protein or polypeptide.

3. The method of claim 2 wherein the protein or polypeptide is detected by immunohistochemical analysis on the tumor sample using an antibody that binds to the protein or polypeptide.

4. The method of claim 2 wherein the protein or polypeptide is detected by ELISA assay using an antibody that specifically binds to the protein or polypeptide.

5. The method of claim 2 wherein the protein or polypeptide is detected using an antibody array comprising an antibody that specifically binds to the protein or polypeptide.

6. The method of claim 2 wherein the protein or polypeptide is detected using an anti-beta-catenin antibody that binds to the protein or polypeptide.

7. The method of claim 1 wherein determining the expression levels of one or more genes selected from the cancer stem cell gene signature is by measuring expression of corresponding mRNA.

8. The method of claim 7 wherein the mRNA is detected using a polynucleotide array comprising a polynucleotide that hybridizes to the mRNA.

9. The method of claim 7 wherein the mRNA is detected using polymerase chain reaction comprising polynucleotide primers to amplify the mRNA.

10. The method of claim 1 wherein the cancer stem cell gene signature comprises genes listed in a table selected from the group consisting of: Table 1A, Table 2A, Table 3A, Table 4A, Table 5A, Table 6A, Table 7A, and Table 8A.

11-17. (canceled)

18. An array comprising polynucleotides hybridizing to cancer stem cell gene signature genes immobilized on a solid surface, wherein said gene signature genes are listed in a table selected from the group consisting of: Table 1A, Table 2A, Table 3A, Table 4A, Table 5A, Table 6A, Table 7A, and Table 8A.

19-25. (canceled)

26. The method of claim 1 wherein the cancer is breast cancer.

27. The method of claim 26 wherein the breast cancer sample is obtained from a fixed, paraffin-embedded biopsy sample.

28. The method of claim 7 wherein the RNA is isolated from a fixed, paraffin-embedded cancer sample.

29. The method of claim 7 wherein the RNA is isolated from core biopsy tissue or fine needle aspirate.

30. The method of claim 2 wherein the protein or polypeptide is detected from a section of a fixed, paraffin-embedded cancer sample.
